SEQUENCE LISTING (30 sequences)

SEQ ID NO: 1 - protein, 16 aa, synthetic construct
  REGION 1..16: note = Def16, cell penetrating peptide
GRCRGFRRRCFCTTHC

SEQ ID NO: 2 - protein, 20 aa, synthetic construct
  REGION 1..20: note = EPAS1
  MOD_RES 1: note = Can be FITC-labeled
  MOD_RES 3: note = Can be FITC-labeled
  MOD_RES 5: note = Can be FITC-labeled
  MOD_RES 14: note = Can be FITC-labeled
  MOD_RES 16..17: note = Can be FITC-labeled
  MOD_RES 20: note = Can be FITC-labeled
ELDLETLAPYIPMDGEDFQL

SEQ ID NO: 3 - protein, 36 aa, synthetic construct
  REGION 1..36: note = Def16-EPAS1
  MOD_RES 1: note = Can be FITC-labeled
  MOD_RES 3: note = Can be FITC-labeled
  MOD_RES 10: note = Can be FITC-labeled
  MOD_RES 12: note = Can be FITC-labeled
  MOD_RES 16..17: note = Can be FITC-labeled
  MOD_RES 19: note = Can be FITC-labeled
  MOD_RES 21: note = Can be FITC-labeled
  MOD_RES 30: note = Can be FITC-labeled
  MOD_RES 32..33: note = Can be FITC-labeled
  MOD_RES 36: note = Can be FITC-labeled
GRCRGFRRRCFCTTHCELDLETLAPYIPMDGEDFQL

SEQ ID NO: 4 - protein, 20 aa, synthetic construct
  REGION 1..20: note = HIF
  MOD_RES 1: note = Can be FITC-labeled
  MOD_RES 3: note = Can be FITC-labeled
  MOD_RES 5: note = Can be FITC-labeled
  MOD_RES 14..16: note = Can be FITC-labeled
  MOD_RES 20: note = Can be FITC-labeled
DLDLEALAPYIPADDDFQLR

SEQ ID NO: 5 - protein, 826 aa, Homo sapiens
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 6 - protein, 870 aa, Homo sapiens
MTADKEKKRSSSERRKEKSRDAARCRRSKETEVFYELAHELPLPHSVSSHLDKASIMRLAISFLRTHKLLSSVCSENESEAEADQQMDNLYLKALEGFIAVVTQDGDMIFLSENISKFMGLTQVELTGHSIFDFTHPCDHEEIRENLSLKNGSGFGKKSKDMSTERDFFMRMKCTVTNRGRTVNLKSATWKVLHCTGQVKVYNNCPPHNSLCGYKEPLLSCLIIMCEPIQHPSHMDIPLDSKTFLSRHSMDMKFTYCDDRITELIGYHPEELLGRSAYEFYHALDSENMTKSHQNLCTKGQVVSGQYRMLAKHGGYVWLETQGTVIYNPRNLQPQCIMCVNYVLSEIEKNDVVFSMDQTESLFKPHLMAMNSIFDSSGKGAVSEKSNFLFTKLKEEPEELAQLAPTPGDAIISLDFGNQNFEESSAYGKAILPPSQPWATELRSHSTQSEAGSLPAFTVPQAAAPGSTTPSATSSSSSCSTPNSPEDYYTSLDNDLKIEVIEKLFAMDTEAKDQCSTQTDFNELDLETLAPYIPMDGEDFQLSPICPEERLLAENPQSTPQHCFSAMTNIFQPLAPVAPHSPFLLDKFQQQLESKKTEPEHRPMSSIFFDAGSKASLPPCCGQASTPLSSMGGRSNTQWPPDPPLHFGPTKWAVGDQRTEFLGAAPLGPPVSPPHVSTFKTRSAKGFGARGPDVLSPAMVALSNKLKLKRQLEYEEQAFQDLSGGDPPGGSTSHLMWKRMKNLRGGSCPLMPDKPLSANVPNDKFTQNPMRGLGHPLRHLPLPQPPSAISPGENSKSRFPPQCYATQYQDYSLSSAHKVSGMASRLLGPSFESYLLPELTRYDCEVNVPVLGSSTLLQGGDLLRALDQAT

SEQ ID NO: 7 - protein, 47 aa, Medicago truncatula
RTCESQSHKFKGPCASDHNCASVCQTERFSGGRCRGFRRRCFCTTHC

SEQ ID NO: 8 - protein, 407 aa, Homo sapiens
MDSPCQPQPLSQALPQLPGSSSEPLEPEPGRARMGVESYLPCPLLPSYHCPGVPSEASAGSGTPRATATSTTASPLRDGFGGQDGGELRPLQSEGAAALVTKGCQRLAAQGARPEAPKRKWAEDGGDAPSPSKRPWARQENQEAEREGGMSCSCSSGSGEASAGLMEEALPSAPERLALDYIVPCMRYYGICVKDSFLGAALGGRVLAEVEALKRGGRLRDGQLVSQRAIPPRSIRGDQIAWVEGHEPGCRSIGALMAHVDAVIRHCAGRLGSYVINGRTKAMVACYPGNGLGYVRHVDNPHGDGRCITCIYYLNQNWDVKVHGGLLQIFPEGRPVVANIEPLFDRLLIFWSDRRNPHEVKPAYATRYAITVWYFDAKERAAAKDKYQLASGQKGVQVPVSQPPTPT

SEQ ID NO: 9 - protein, 426 aa, Homo sapiens
MANDSGGPGGPSPSERDRQYCELCGKMENLLRCSRCRSSFYCCKEHQRQDWKKHKLVCQGSEGALGHGVGPHQHSGPAPPAAVPPPRAGAREPRKAAARRDNASGDAAKGKVKAKPPADPAAAASPCRAAAGGQGSAVAAEAEPGKEEPPARSSLFQEKANLYPPSNTPGDALSPGGGLRPNGQTKPLPALKLALEYIVPCMNKHGICVVDDFLGKETGQQIGDEVRALHDTGKFTDGQLVSQKSDSSKDIRGDKITWIEGKEPGCETIGLLMSSMDDLIRHCNGKLGSYKINGRTKAMVACYPGNGTGYVRHVDNPNGDGRCVTCIYYLNKDWDAKVSGGILRIFPEGKAQFADIEPKFDRLLFFWSDRRNPHEVQPAYATRYAITVWYFDADERARAKVKYLTGEKGVRVELNKPSDSVGKDVF

SEQ ID NO: 10 - protein, 239 aa, Homo sapiens
MPLGHIMRLDLEKIALEYIVPCLHEVGFCYLDNFLGEVVGDCVLERVKQLHCTGALRDGQLAGPRAGVSKRHLRGDQITWIGGNEEGCEAISFLLSLIDRLVLYCGSRLGKYYVKERSKAMVACYPGNGTGYVRHVDNPNGDGRCITCIYYLNKNWDAKLHGGILRIFPEGKSFIADVEPIFDRLLFFWSDRRNPHEVQPSYATRYAMTVWYFDAEERAEAKKKFRNLTRKTESALTED

SEQ ID NO: 11 - protein, 789 aa, Homo sapiens
MAATTANPEMTSDVPSLGPAIASGNSGPGIQGGGAIVQRAIKRRPGLDFDDDGEGNSKFLRCDDDQMSNDKERFARSDDEQSSADKERLARENHSEIERRRRNKMTAYITELSDMVPTCSALARKPDKLTILRMAVSHMKSLRGTGNTSTDGSYKPSFLTDQELKHLILEAADGFLFIVSCETGRVVYVSDSVTPVLNQPQSEWFGSTLYDQVHPDDVDKLREQLSTSENALTGRILDLKTGTVKKEGQQSSMRMCMGSRRSFICRMRCGSSSVDPVSVNRLSFVRNRCRNGLGSVKDGEPHFVVVHCTGYIKAWPPAGVSLPDDDPEAGQGSKFCLVAIGRLQVTSSPNCTDMSNVCQPTEFISRHNIEGIFTFVDHRCVATVGYQPQELLGKNIVEFCHPEDQQLLRDSFQQVVKLKGQVLSVMFRFRSKNQEWLWMRTSSFTFQNPYSDEIEYIICTNTNVKNSSQEPRPTLSNTIQRPQLGPTANLPLEMGSGQLAPRQQQQQTELDMVPGRDGLASYNHSQVVQPVTTTGPEHSKPLEKSDGLFAQDRDPRFSEIYHNINADQSKGISSSTVPATQQLFSQGNTFPPTPRPAENFRNSGLAPPVTIVQPSASAGQMLAQISRHSNPTQGATPTWTPTTRSGFSAQQVATQATAKTRTSQFGVGSFQTPSSFSSMSLPGAPTASPGAAAYPSLTNRGSNFAPETGQTAGQFQTRTAEGVGVWPQWQGQQPHHRSSSSEQHVQQPPAQQPGQPEVFQEMLSMLGDQSNSYNNEEFPDLTMFPPFSE

SEQ ID NO: 12 - DNA, 3946 bp, Homo sapiens
  source 1..3946: mol_type = genomic DNA
agtgcacagtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcattacctaaagcagtctatttatattttctacatctaattttagaagcctggctacaatactgcacaaacttggttagttcaattttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccagg

SEQ ID NO: 13 - protein, 17 aa, synthetic construct
  REGION 1..17: note = amino acids 556-572 of NP_001521.1; C-terminal VHL recognition site
DLDLEMLAPYIPMDDDF

SEQ ID NO: 14 - protein, 47 aa, synthetic construct
  REGION 1..47: note = amino acids 496-542 of NP_001421.2
LKIEVIEKLFAMDTEAKDQCSTQTDFNELDLETLAPYIPMDGEDFQL

SEQ ID NO: 15 - protein, 246 aa, synthetic construct
  REGION 1..246: note = PHD2 partial sequence recombinantly expressed
PNGQTKPLPALKLALEYIVPCMNKHGICVVDDFLGKETGQQIGDEVRALHDTGKFTDGQLVSQKSDSSKDIRGDKITWIEGKEPGCETIGLLMSSMDDLIRHCNGKLGSYKINGRTKAMVACYPGNGTGYVRHVDNPNGDGRCVTCIYYLNKDWDAKVSGGILRIFPEGKAQFADIEPKFDRLLFFWSDRRNPHEVQPAYATRYAITVWYFDADERARAKVKYLTGEKGVRVELNKPSDSVGKDVF

SEQ ID NO: 16 - protein, 759 aa, synthetic construct
  REGION 1..759: note = cDNA cloned into PGEX-6P-1 plasmid containing an N-terminus GST tag -
GGTAGCCACATGGCGAGCCCGAATGGTCAAACCAAACCGCTGCCGGCGCTGAAACTGGCGCTGGAGTACATCGTTCCGTGCATGAATAAGCACGGTATCTGCGTGGTTGACGATTTTCTGGGCAAAGAAACCGGTCAGCAAATTGGCGATGAAGTGCGTGCGCTGCACGATACCGGCAAGTTCACCGACGGCCAGCTGGTTAGCCAAAAGAGCGACAGCAGCAAAGATATCCGTGGTGACAAGATCACCTGGATTGAGGGTAAAGAACCGGGCTGCGAAACCATCGGCCTGCTGATGAGCAGCATGGACGATCTGATTCGTCACTGCAACGGCAAGCTGGGTAGCTACAAAATTAACGGTCGTACCAAAGCGATGGTGGCGTGCTACCCGGGTAACGGTACCGGTTATGTGCGTCACGTTGATAACCCGAACGGCGACGGTCGTTGCGTGACCTGCATCTACTATCTGAACAAGGACTGGGATGCGAAAGTTAGCGGTGGCATCCTGCGTATTTTCCCGGAGGGCAAGGCGCAGTTTGCGGATATTGAACCGAAATTCGACCGTCTGCTGTTCTTTTGGAGCGACCGTCGTAACCCGCACGAAGTGCAACCGGCGTACGCGACCCGTTATGCGATCACCGTTTGGTACTTTGACGCGGATGAGCGTGCGCGTGCGAAGGTTAAATATCTGACCGGTGAAAAAGGTGTGCGTGTGGAACTGAATAAGCCGAGCGACAGCGTGGGCAAGGATGTTTTCTAA

SEQ ID NO: 17 - DNA, 5716 bp, Homo sapiens
  source 1..5716: mol_type = genomic DNA
gcagacgggagtttctcctcggggtcggagcaggaggcacgcggagtgtgaggccacgcatgagcggacgctaaccccctccccagccacaaagagtctacatgtctagggtctagacatgttcagctttgtggacctccggctcctgctcctcttagcggccaccgccctcctgacgcacggccaagaggaaggccaagtcgagggccaagacgaagacatcccaccaatcacctgcgtacagaacggcctcaggtaccatgaccgagacgtgtggaaacccgagccctgccggatctgcgtctgcgacaacggcaaggtgttgtgcgatgacgtgatctgtgacgagaccaagaactgccccggcgccgaagtccccgagggcgagtgctgtcccgtctgccccgacggctcagagtcacccaccgaccaagaaaccaccggcgtcgagggacccaagggagacactggcccccgaggcccaaggggacccgcaggcccccctggccgagatggcatccctggacagcctggacttcccggaccccccggaccccccggacctcccggaccccctggcctcggaggaaactttgctccccagctgtcttatggctatgatgagaaatcaaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggaaaccctggtgctgatggacagcctggtgctaaaggtgccaatggtgctcctggtattgctggtgctcctggcttccctggtgcccgaggcccctctggaccccagggccccggcggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccctgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggtccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgactggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgctaaaggcgatgctggtccccctggccctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggtcctgctggtgctcctggtactcccgggcctcaaggtattgctggacagcgtggtgtggtcggcctgcctggtcagagaggagagagaggcttccctggtcttcctggcccctctggtgaacctggcaaacaaggtccctctggagcaagtggtgaacgtggtccccctggtcccatgggcccccctggattggctggaccccctggtgaatctggacgtgagggggctcctggtgccgaaggttcccctggacgagacggttctcctggcgccaagggtgaccgtggtgagaccggccccgctggaccccctggtgctcctggtgctcctggtgcccctggccccgttggccctgctggcaagagtggtgatcgtggtgagactggtcctgctggtcccgccggtcctgtcggccctgttggcgcccgtggccccgccggaccccaaggcccccgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggcagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggccatccagctgaccttcctgcgcctgatgtccaccgaggcctcccagaacatcacctaccactgcaagaacagcgtggcctacatggaccagcagactggcaacctcaagaaggccctgctcctccagggctccaacgagatcgagatccgcgccgagggcaacagccgcttcacctacagcgtcactgtcgatggctgcacgagtcacaccggagcctggggcaagacagtgattgaatacaaaaccaccaagacctcccgcctgcccatcatcgatgtggcccccttggacgttggtgccccagaccaggaattcggcttcgacgttggccctgtctgcttcctgtaaactccctccatcccaacctggctccctcccacccaaccaactttccccccaacccggaaacagacaagcaacccaaactgaaccccctcaaaagccaaaaaatgggagacaatttcacatggactttggaaaatatttttttcctttgcattcatctctcaaacttagtttttatctttgaccaaccgaacatgaccaaaaaccaaaagtgcattcaaccttaccaaaaaaaaaaaaaaaaaaagaataaataaataactttttaaaaaaggaagcttggtccacttgcttgaagacccatgcgggggtaagtccctttctgcccgttgggcttatgaaaccccaatgctgccctttctgctcctttctccacaccccccttggggcctcccctccactccttcccaaatctgtctccccagaagacacaggaaacaatgtattgtctgcccagcaatcaaaggcaatgctcaaacacccaagtggcccccaccctcagcccgctcctgcccgcccagcacccccaggccctgggggacctggggttctcagactgccaaagaagccttgccatctggcgctcccatggctcttgcaacatctccccttcgtttttgagggggtcatgccgggggagccaccagcccctcactgggttcggaggagagtcaggaagggccacgacaaagcagaaacatcggatttggggaacgcgtgtcaatcccttgtgccgcagggctgggcgggagagactgttctgttccttgtgtaactgtgttgctgaaagactacctcgttcttgtcttgatgtgtcaccggggcaactgcctgggggcggggatgggggcagggtggaagcggctccccattttataccaaaggtgctacatctatgtgatgggtggggtggggagggaatcactggtgctatagaaattgagatgcccccccaggccagcaaatgttcctttttgttcaaagtctatttttattccttgatatttttctttttttttttttttttttgtggatggggacttgtgaatttttctaaaggtgctatttaacatgggaggagagcgtgtgcggctccagcccagcccgctgctcactttccaccctctctccacctgcctctggcttctcaggcctctgctctccgacctctctcctctgaaaccctcctccacagctgcagcccatcctcccggctccctcctagtctgtcctgcgtcctctgtccccgggtttcagagacaacttcccaaagcacaaagcagtttttccccctaggggtgggaggaagcaaaagactctgtacctattttgtatgtgtataataatttgagatgtttttaattattttgattgctggaataaagcatgtggaaatgacccaaa

SEQ ID NO: 18 - protein, 1398 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 19 - DNA, 5644 bp, Homo sapiens
  source 1..5644: mol_type = genomic DNA
gcagacgggagtttctcctcggggtcggagcaggaggcacgcggagtgtgaggccacgcatgagcggacgctaaccccctccccagccacaaagagtctacatgtctagggtctagacatgttcagctttgtggacctccggctcctgctcctcttagcggccaccgccctcctgacgcacggccaagaggaaggccaagtcgagggccaagacgaagacatcccaccaatcacctgcgtacagaacggcctcaggtaccatgaccgagacgtgtggaaacccgagccctgccggatctgcgtctgcgacaacggcaaggtgttgtgcgatgacgtgatctgtgacgagaccaagaactgccccggcgccgaagtccccgagggcgagtgctgtcccgtctgccccgacggctcagagtcacccaccgaccaagaaaccaccggcgtcgagggacccaagggagacactggcccccgaggcccaaggggacccgcaggcccccctggccgagatggcatccctggacagcctggacttcccggaccccccggaccccccggacctcccggaccccctggcctcggaggaaactttgctccccagctgtcttatggctatgatgagaaatcaaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggtgctcgtggaaatgatggtgctactggtgctgccgggccccctggtcccaccggccccgctggtcctcctggcttccctggtgctgttggtgctaagggtgaagctggtccccaagggccccgaggctctgaaggtccccagggtgtgcgtggtgagcctggcccccctggccctgctggtgctgctggccctgctggaaaccctggtgctgatggacagcctggtgctaaaggtgccaatggtgctcctggtattgctggtgctcctggcttccctggtgcccgaggcccctctggaccccagggccccggcggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccctgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggtccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgactggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgctaaaggcgatgctggtccccctggccctgccggacccgctggaccccctggccccattggtaatgttggtgctcctggagccaaaggtgctcgcggcagcgctggtccccctggtgctactggtttccctggtgctgctggccgagtcggtcctcctggcccctctggtgaacctggcaaacaaggtccctctggagcaagtggtgaacgtggtccccctggtcccatgggcccccctggattggctggaccccctggtgaatctggacgtgagggggctcctggtgccgaaggttcccctggacgagacggttctcctggcgccaagggtgaccgtggtgagaccggccccgctggaccccctggtgctcctggtgctcctggtgcccctggccccgttggccctgctggcaagagtggtgatcgtggtgagactggtcctgctggtcccgccggtcctgtcggccctgttggcgcccgtggccccgccggaccccaaggcccccgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggcagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggccatccagctgaccttcctgcgcctgatgtccaccgaggcctcccagaacatcacctaccactgcaagaacagcgtggcctacatggaccagcagactggcaacctcaagaaggccctgctcctccagggctccaacgagatcgagatccgcgccgagggcaacagccgcttcacctacagcgtcactgtcgatggctgcacgagtcacaccggagcctggggcaagacagtgattgaatacaaaaccaccaagacctcccgcctgcccatcatcgatgtggcccccttggacgttggtgccccagaccaggaattcggcttcgacgttggccctgtctgcttcctgtaaactccctccatcccaacctggctccctcccacccaaccaactttccccccaacccggaaacagacaagcaacccaaactgaaccccctcaaaagccaaaaaatgggagacaatttcacatggactttggaaaatatttttttcctttgcattcatctctcaaacttagtttttatctttgaccaaccgaacatgaccaaaaaccaaaagtgcattcaaccttaccaaaaaaaaaaaaaaaaaaagaataaataaataactttttaaaaaaggaagcttggtccacttgcttgaagacccatgcgggggtaagtccctttctgcccgttgggcttatgaaaccccaatgctgccctttctgctcctttctccacaccccccttggggcctcccctccactccttcccaaatctgtctccccagaagacacaggaaacaatgtattgtctgcccagcaatcaaaggcaatgctcaaacacccaagtggcccccaccctcagcccgctcctgcccgcccagcacccccaggccctgggggacctggggttctcagactgccaaagaagccttgccatctggcgctcccatggctcttgcaacatctccccttcgtttttgagggggtcatgccgggggagccaccagcccctcactgggttcggaggagagtcaggaagggccacgacaaagcagaaacatcggatttggggaacgcgtgtcaatcccttgtgccgcagggctgggcgggagagactgttctgttccttgtgtaactgtgttgctgaaagactacctcgttcttgtcttgatgtgtcaccggggcaactgcctgggggcggggatgggggcagggtggaagcggctccccattttataccaaaggtgctacatctatgtgatgggtggggtggggagggaatcactggtgctatagaaattgagatgcccccccaggccagcaaatgttcctttttgttcaaagtctatttttattccttgatatttttctttttttttttttttttttgtggatggggacttgtgaatttttctaaaggtgctatttaacatgggaggagagcgtgtgcggctccagcccagcccgctgctcactttccaccctctctccacctgcctctggcttctcaggcctctgctctccgacctctctcctctgaaaccctcctccacagctgcagcccatcctcccggctccctcctagtctgtcctgcgtcctctgtccccgggtttcagagacaacttcccaaagcacaaagcagtttttccccctaggggtgggaggaagcaaaagactctgtacctattttgtatgtgtataataatttgagatgtttttaattattttgattgctggaataaagcatgtggaaatgacccaaa

SEQ ID NO: 20 - protein, 1374 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 21 - DNA, 4996 bp, Homo sapiens
  source 1..4996: mol_type = genomic DNA
gcagacgggagtttctcctcggggtcggagcaggaggcacgcggagtgtgaggccacgcatgagcggacgctaaccccctccccagccacaaagagtctacatgtctagggtctagacatgttcagctttgtggacctccggctcctgctcctcttagcggccaccgccctcctgacgcacggccaagaggaaggccaagtcgagggccaagacgaagacatcccaccaatcacctgcgtacagaacggcctcaggtaccatgaccgagacgtgtggaaacccgagccctgccggatctgcgtctgcgacaacggcaaggtgttgtgcgatgacgtgatctgtgacgagaccaagaactgccccggcgccgaagtccccgagggcgagtgctgtcccgtctgccccgacggctcagagtcacccaccgaccaagaaaccaccggcgtcgagggacccaagggagacactggcccccgaggcccaaggggacccgcaggcccccctggccgagatggcatccctggacagcctggacttcccggaccccccggaccccccggacctcccggaccccctggcctcggaggaaactttgctccccagctgtcttatggctatgatgagaaatcaaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggtccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgactggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgctaaaggcgatgctggtccccctggccctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggtcctgctggtgctcctggtactcccgggcctcaaggtattgctggacagcgtggtgtggtcggcctgcctggtcagagaggagagagaggcttccctggtcttcctggcccctctggtgaacctggcaaacaaggtccctctggagcaagtggtgaacgtggtccccctggtcccatgggcccccctggattggctggaccccctggtgaatctggacgtgagggggctcctggtgccgaaggttcccctggacgagacggttctcctggcgccaagggtgaccgtggtgagaccggccccgctggaccccctggtgctcctggtgctcctggtgcccctggccccgttggccctgctggcaagagtggtgatcgtggtgagactggtcctgctggtcccgccggtcctgtcggccctgttggcgcccgtggccccgccggaccccaaggcccccgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggcagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggccatccagctgaccttcctgcgcctgatgtccaccgaggcctcccagaacatcacctaccactgcaagaacagcgtggcctacatggaccagcagactggcaacctcaagaaggccctgctcctccagggctccaacgagatcgagatccgcgccgagggcaacagccgcttcacctacagcgtcactgtcgatggctgcacgagtcacaccggagcctggggcaagacagtgattgaatacaaaaccaccaagacctcccgcctgcccatcatcgatgtggcccccttggacgttggtgccccagaccaggaattcggcttcgacgttggccctgtctgcttcctgtaaactccctccatcccaacctggctccctcccacccaaccaactttccccccaacccggaaacagacaagcaacccaaactgaaccccctcaaaagccaaaaaatgggagacaatttcacatggactttggaaaatatttttttcctttgcattcatctctcaaacttagtttttatctttgaccaaccgaacatgaccaaaaaccaaaagtgcattcaaccttaccaaaaaaaaaaaaaaaaaaagaataaataaataactttttaaaaaaggaagcttggtccacttgcttgaagacccatgcgggggtaagtccctttctgcccgttgggcttatgaaaccccaatgctgccctttctgctcctttctccacaccccccttggggcctcccctccactccttcccaaatctgtctccccagaagacacaggaaacaatgtattgtctgcccagcaatcaaaggcaatgctcaaacacccaagtggcccccaccctcagcccgctcctgcccgcccagcacccccaggccctgggggacctggggttctcagactgccaaagaagccttgccatctggcgctcccatggctcttgcaacatctccccttcgtttttgagggggtcatgccgggggagccaccagcccctcactgggttcggaggagagtcaggaagggccacgacaaagcagaaacatcggatttggggaacgcgtgtcaatcccttgtgccgcagggctgggcgggagagactgttctgttccttgtgtaactgtgttgctgaaagactacctcgttcttgtcttgatgtgtcaccggggcaactgcctgggggcggggatgggggcagggtggaagcggctccccattttataccaaaggtgctacatctatgtgatgggtggggtggggagggaatcactggtgctatagaaattgagatgcccccccaggccagcaaatgttcctttttgttcaaagtctatttttattccttgatatttttctttttttttttttttttttgtggatggggacttgtgaatttttctaaaggtgctatttaacatgggaggagagcgtgtgcggctccagcccagcccgctgctcactttccaccctctctccacctgcctctggcttctcaggcctctgctctccgacctctctcctctgaaaccctcctccacagctgcagcccatcctcccggctccctcctagtctgtcctgcgtcctctgtccccgggtttcagagacaacttcccaaagcacaaagcagtttttccccctaggggtgggaggaagcaaaagactctgtacctattttgtatgtgtataataatttgagatgtttttaattattttgattgctggaataaagcatgtggaaatgacccaaa

SEQ ID NO: 22 - protein, 1158 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 23 - DNA, 5072 bp, Homo sapiens
  source 1..5072: mol_type = genomic DNA
agcaccacggcagcaggaggtttcggctaagttggaggtactggccacgactgcatgcccgcgcccgccaggtgatacctccgccggtgacccaggggctctgcgacacaaggagtctgcatgtctaagtgctagacatgctcagctttgtggatacgcggactttgttgctgcttgcagtaaccttatgcctagcaacatgccaatctttacaagaggaaactgtaagaaagggcccagccggagatagaggaccacgtggagaaaggggtccaccaggccccccaggcagagatggtgaagatggtcccacaggccctcctggtccacctggtcctcctggcccccctggtctcggtgggaactttgctgctcagtatgatggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgtgttggtgcccctggcccagctggtgcccgtggcagtgatggaagtgtgggtcccgtgggtcctgctggtcccattgggtctgctggccctccaggcttcccaggtgcccctggccccaagggtgaaattggagctgttggtaacgctggtcctgctggtcccgccggtccccgtggtgaagtgggtcttccaggcctctccggccccgttggacctcctggtaatcctggagcaaacggccttactggtgccaagggtgctgctggccttcccggcgttgctggggctcccggcctccctggaccccgcggtattcctggccctgttggtgctgccggtgctactggtgccagaggacttgttggtgagcctggtccagctggctccaaaggagagagcggtaacaagggtgagcccggctctgctgggccccaaggtcctcctggtcccagtggtgaagaaggaaagagaggccctaatggggaagctggatctgccggccctccaggacctcctgggctgagaggtagtcctggttctcgtggtcttcctggagctgatggcagagctggcgtcatgggccctcctggtagtcgtggtgcaagtggccctgctggagtccgaggacctaatggagatgctggtcgccctggggagcctggtctcatgggacccagaggtcttcctggttcccctggaaatatcggccccgctggaaaagaaggtcctgtcggcctccctggcatcgacggcaggcctggcccaattggcccagctggagcaagaggagagcctggcaacattggattccctggacccaaaggccccactggtgatcctggcaaaaacggtgataaaggtcatgctggtcttgctggtgctcggggtgctccaggtcctgatggaaacaatggtgctcagggacctcctggaccacagggtgttcaaggtggaaaaggtgaacagggtccccctggtcctccaggcttccagggtctgcctggcccctcaggtcccgctggtgaagttggcaaaccaggagaaaggggtctccatggtgagtttggtctccctggtcctgctggtccaagaggggaacgcggtcccccaggtgagagtggtgctgccggtcctactggtcctattggaagccgaggtccttctggacccccagggcctgatggaaacaagggtgaacctggtgtggttggtgctgtgggcactgctggtccatctggtcctagtggactcccaggagagaggggtgctgctggcatacctggaggcaagggagaaaagggtgaacctggtctcagaggtgaaattggtaaccctggcagagatggtgctcgtggtgctcctggtgctgtaggtgcccctggtcctgctggagccacaggtgaccggggcgaagctggggctgctggtcctgctggtcctgctggtcctcggggaagccctggtgaacgtggtgaggtcggtcctgctggccccaatggatttgctggtcctgctggtgctgctggtcaacctggtgctaaaggagaaagaggagccaaagggcctaagggtgaaaacggtgttgttggtcccacaggccccgttggagctgctggcccagctggtccaaatggtccccccggtcctgctggaagtcgtggtgatggaggcccccctggtatgactggtttccctggtgctgctggacggactggtcccccaggaccctctggtatttctggccctcctggtccccctggtcctgctgggaaagaagggcttcgtggtcctcgtggtgaccaaggtccagttggccgaactggagaagtaggtgcagttggtccccctggcttcgctggtgagaagggtccctctggagaggctggtactgctggacctcctggcactccaggtcctcagggtcttcttggtgctcctggtattctgggtctccctggctcgagaggtgaacgtggtctaccaggtgttgctggtgctgtgggtgaacctggtcctcttggcattgccggccctcctggggcccgtggtcctcctggtgctgtgggtagtcctggagtcaacggtgctcctggtgaagctggtcgtgatggcaaccctgggaacgatggtcccccaggtcgcgatggtcaacccggacacaagggagagcgcggttaccctggcaatattggtcccgttggtgctgcaggtgcacctggtcctcatggccccgtgggtcctgctggcaaacatggaaaccgtggtgaaactggtccttctggtcctgttggtcctgctggtgctgttggcccaagaggtcctagtggcccacaaggcattcgtggcgataagggagagcccggtgaaaaggggcccagaggtcttcctggcttaaagggacacaatggattgcaaggtctgcctggtatcgctggtcaccatggtgatcaaggtgctcctggctccgtgggtcctgctggtcctaggggccctgctggtccttctggccctgctggaaaagatggtcgcactggacatcctggtacagttggacctgctggcattcgaggccctcagggtcaccaaggccctgctggcccccctggtccccctggccctcctggacctccaggtgtaagcggtggtggttatgactttggttacgatggagacttctacagggctgaccagcctcgctcagcaccttctctcagacccaaggactatgaagttgatgctactctgaagtctctcaacaaccagattgagacccttcttactcctgaaggctctagaaagaacccagctcgcacatgccgtgacttgagactcagccacccagagtggagcagtggttactactggattgaccctaaccaaggatgcactatggatgctatcaaagtatactgtgatttctctactggcgaaacctgtatccgggcccaacctgaaaacatcccagccaagaactggtataggagctccaaggacaagaaacacgtctggctaggagaaactatcaatgctggcagccagtttgaatataatgtagaaggagtgacttccaaggaaatggctacccaacttgccttcatgcgcctgctggccaactatgcctctcagaacatcacctaccactgcaagaacagcattgcatacatggatgaggagactggcaacctgaaaaaggctgtcattctacagggctctaatgatgttgaacttgttgctgagggcaacagcaggttcacttacactgttcttgtagatggctgctctaaaaagacaaatgaatggggaaagacaatcattgaatacaaaacaaataagccatcacgcctgcccttccttgatattgcacctttggacatcggtggtgctgaccaggaattctttgtggacattggcccagtctgtttcaaataaatgaactcaatctaaattaaaaaagaaagaaatttgaaaaaactttctctttgccatttcttcttcttcttttttaactgaaagctgaatccttccatttcttctgcacatctacttgcttaaattgtgggcaaaagagaaaaagaaggattgatcagagcattgtgcaatacagtttcattaactccttcccccgctcccccaaaaatttgaatttttttttcaacactcttacacctgttatggaaaatgtcaacctttgtaagaaaaccaaaataaaaattgaaaaataaaaaccataaacatttgcaccacttgtggcttttgaatatcttccacagagggaagtttaaaacccaaacttccaaaggtttaaactacctcaaaacactttcccatgagtgtgatccacattgttaggtgctgacctagacagagatgaactgaggtccttgttttgttttgttcataatacaaaggtgctaattaatagtatttcagatacttgaagaatgttgatggtgctagaagaatttgagaagaaatactcctgtattgagttgtatcgtgtggtgtattttttaaaaaatttgatttagcattcatattttccatcttattcccaattaaaagtatgcagattatttgcccaaatcttcttcagattcagcatttgttctttgccagtctcattttcatcttcttccatggttccacagaagctttgtttcttgggcaagcagaaaaattaaattgtacctattttgtatatgtgagatgtttaaataaattgtgaaaaaaatgaaataaagcatgtttggttttccaaaagaa

SEQ ID NO: 24 - protein, 1366 aa, Homo sapiens
MLSFVDTRTLLLLAVTLCLATCQSLQEETVRKGPAGDRGPRGERGPPGPPGRDGEDGPTGPPGPPGPPGPPGLGGNFAAQYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPAGPAGPRGEVGLPGLSGPVGPPGNPGANGLTGAKGAAGLPGVAGAPGLPGPRGIPGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPPGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAPGAVGAPGPAGATGDRGEAGAAGPAGPAGPRGSPGERGEVGPAGPNGFAGPAGAAGQPGAKGERGAKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGLKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMDAIKVYCDFSTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADQEFFVDIGPVCFK

SEQ ID NO: 25 - DNA, 5490 bp, Homo sapiens
  source 1..5490: mol_type = genomic DNA
ggctgagttttatgacgggcccggtgctgaagggcagggaacaacttgatggtgctactttgaactgcttttcttttctcctttttgcacaaagagtctcatgtctgatatttagacatgatgagctttgtgcaaaaggggagctggctacttctcgctctgcttcatcccactattattttggcacaacaggaagctgttgaaggaggatgttcccatcttggtcagtcctatgcggatagagatgtctggaagccagaaccatgccaaatatgtgtctgtgactcaggatccgttctctgcgatgacataatatgtgacgatcaagaattagactgccccaacccagaaattccatttggagaatgttgtgcagtttgcccacagcctccaactgctcctactcgccctcctaatggtcaaggacctcaaggccccaagggagatccaggccctcctggtattcctgggagaaatggtgaccctggtattccaggacaaccagggtcccctggttctcctggcccccctggaatctgtgaatcatgccctactggtcctcagaactattctccccagtatgattcatatgatgtcaagtctggagtagcagtaggaggactcgcaggctatcctggaccagctggccccccaggccctcccggtccccctggtacatctggtcatcctggttcccctggatctccaggataccaaggaccccctggtgaacctgggcaagctggtccttcaggccctccaggacctcctggtgctataggtccatctggtcctgctggaaaagatggagaatcaggtagacccggacgacctggagagcgaggattgcctggacctccaggtatcaaaggtccagctgggatacctggattccctggtatgaaaggacacagaggcttcgatggacgaaatggagaaaagggtgaaacaggtgctcctggattaaagggtgaaaatggtcttccaggcgaaaatggagctcctggacccatgggtccaagaggggctcctggtgagcgaggacggccaggacttcctggggctgcaggtgctcggggtaatgacggtgctcgaggcagtgatggtcaaccaggccctcctggtcctcctggaactgccggattccctggatcccctggtgctaagggtgaagttggacctgcagggtctcctggttcaaatggtgcccctggacaaagaggagaacctggacctcagggacacgctggtgctcaaggtcctcctggccctcctgggattaatggtagtcctggtggtaaaggcgaaatgggtcccgctggcattcctggagctcctggactgatgggagcccggggtcctccaggaccagccggtgctaatggtgctcctggactgcgaggtggtgcaggtgagcctggtaagaatggtgccaaaggagagcccggaccacgtggtgaacgcggtgaggctggtattccaggtgttccaggagctaaaggcgaagatggcaaggatggatcacctggagaacctggtgcaaatgggcttccaggagctgcaggagaaaggggtgcccctgggttccgaggacctgctggaccaaatggcatcccaggagaaaagggtcctgctggagagcgtggtgctccaggccctgcagggcccagaggagctgctggagaacctggcagagatggcgtccctggaggtccaggaatgaggggcatgcccggaagtccaggaggaccaggaagtgatgggaaaccagggcctcccggaagtcaaggagaaagtggtcgaccaggtcctcctgggccatctggtccccgaggtcagcctggtgtcatgggcttccccggtcctaaaggaaatgatggtgctcctggtaagaatggagaacgaggtggccctggaggacctggccctcagggtcctcctggaaagaatggtgaaactggacctcagggacccccagggcctactgggcctggtggtgacaaaggagacacaggaccccctggtccacaaggattacaaggcttgcctggtacaggtggtcctccaggagaaaatggaaaacctggggaaccaggtccaaagggtgatgccggtgcacctggagctccaggaggcaagggtgatgctggtgcccctggtgaacgtggacctcctggattggcaggggccccaggacttagaggtggagctggtccccctggtcccgaaggaggaaagggtgctgctggtcctcctgggccacctggtgctgctggtactcctggtctgcaaggaatgcctggagaaagaggaggtcttggaagtcctggtccaaagggtgacaagggtgaaccaggcggtccaggtgctgatggtgtcccagggaaagatggcccaaggggtcctactggtcctattggtcctcctggcccagctggccagcctggagataagggtgaaggtggtgcccccggacttccaggtatagctggacctcgtggtagccctggtgagagaggtgaaactggccctccaggacctgctggtttccctggtgctcctggacagaatggtgaacctggtggtaaaggagaaagaggggctccgggtgagaaaggtgaaggaggccctcctggagttgcaggaccccctggaggttctggacctgctggtcctcctggtccccaaggtgtcaaaggtgaacgtggcagtcctggtggacctggtgctgctggcttccctggtgctcgtggtcttcctggtcctcctggtagtaatggtaacccaggacccccaggtcccagcggttctccaggcaaggatgggcccccaggtcctgcgggtaacactggtgctcctggcagccctggagtgtctggaccaaaaggtgatgctggccaaccaggagagaagggatcgcctggtgcccagggcccaccaggagctccaggcccacttgggattgctgggatcactggagcacggggtcttgcaggaccaccaggcatgccaggtcctaggggaagccctggccctcagggtgtcaagggtgaaagtgggaaaccaggagctaacggtctcagtggagaacgtggtccccctggaccccagggtcttcctggtctggctggtacagctggtgaacctggaagagatggaaaccctggatcagatggtcttccaggccgagatggatctcctggtggcaagggtgatcgtggtgaaaatggctctcctggtgcccctggcgctcctggtcatccaggcccacctggtcctgtcggtccagctggaaagagtggtgacagaggagaaagtggccctgctggccctgctggtgctcccggtcctgctggttcccgaggtgctcctggtcctcaaggcccacgtggtgacaaaggtgaaacaggtgaacgtggagctgctggcatcaaaggacatcgaggattccctggtaatccaggtgccccaggttctccaggccctgctggtcagcagggtgcaatcggcagtccaggacctgcaggccccagaggacctgttggacccagtggacctcctggcaaagatggaaccagtggacatccaggtcccattggaccaccagggcctcgaggtaacagaggtgaaagaggatctgagggctccccaggccacccagggcaaccaggccctcctggacctcctggtgcccctggtccttgctgtggtggtgttggagccgctgccattgctgggattggaggtgaaaaagctggcggttttgccccgtattatggagatgaaccaatggatttcaaaatcaacaccgatgagattatgacttcactcaagtctgttaatggacaaatagaaagcctcattagtcctgatggttctcgtaaaaaccccgctagaaactgcagagacctgaaattctgccatcctgaactcaagagtggagaatactgggttgaccctaaccaaggatgcaaattggatgctatcaaggtattctgtaatatggaaactggggaaacatgcataagtgccaatcctttgaatgttccacggaaacactggtggacagattctagtgctgagaagaaacacgtttggtttggagagtccatggatggtggttttcagtttagctacggcaatcctgaacttcctgaagatgtccttgatgtgcatctggcattccttcgacttctctccagccgagcttcccagaacatcacatatcactgcaaaaatagcattgcatacatggatcaggccagtggaaatgtaaagaaggccctgaagctgatggggtcaaatgaaggtgaattcaaggctgaaggaaatagcaaattcacctacacagttctggaggatggttgcacgaaacacactggggaatggagcaaaacagtctttgaatatcgaacacgcaaggctgtgagactacctattgtagatattgcaccctatgacattggtggtcctgatcaagaatttggtgtggacgttggccctgtttgctttttataaaccaaactctatctgaaatcccaacaaaaaaaatttaactccatatgtgttcctcttgttctaatcttgtcaaccagtgcaagtgaccgacaaaattccagttatttatttccaaaatgtttggaaacagtataatttgacaaagaaaaatgatacttctctttttttgctgttccaccaaatacaattcaaatgctttttgttttatttttttaccaattccaatttcaaaatgtctcaatggtgctataataaataaacttcaacactctttatgataacaacactgtgttatattctttgaatcctagcccatctgcagagcaatgactgtgctcaccagtaaaagataacctttctttctgaaatagtcaaatacgaaattagaaaagccctccctattttaactacctcaactggtcagaaacacagattgtattctatgagtcccagaagatgaaaaaaattttatacgttgataaaacttataaatttcattgattaatctcctggaagattggtttaaaaagaaaagtgtaatgcaagaatttaaagaaatatttttaaagccacaattattttaatattggatatcaactgcttgtaaaggtgctcctcttttttcttgtcattgctggtcaagattactaatatttgggaaggctttaaagacgcatgttatggtgctaatgtactttcacttttaaactctagatcagaattgttgacttgcattcagaacataaatgcacaaaatctgtacatgtctcccatcagaaagattcattggcatgccacaggggattctcctccttcatcctgtaaaggtcaacaataaaaaccaaattatggggctgcttttgtcacactagcatagagaatgtgttgaaatttaactttgtaagcttgtatgtggttgttgatcttttttttccttacagacacccataataaaatatcatattaaaattc

SEQ ID NO: 26 - protein, 1466 aa, Homo sapiens
MMSFVQKGSWLLLALLHPTIILAQQEAVEGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGIPGQPGSPGSPGPPGICESCPTGPQNYSPQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYGDEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVHLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 27 - protein, 249 aa, synthetic construct
  REGION 1..249: note = cDNA cloned into PGEX-6P-1 plasmid containing an N-terminus GST tag -
MASPNGQTKPLPALKLALEYIVPCMNKHGICVVDDFLGKETGQQIGDEVRALHDTGKFTDGQLVSQKSDSSKDIRGDKITWIEGKEPGCETIGLLMSSMDDLIRHCNGKLGSYKINGRTKAMVACYPGNGTGYVRHVDNPNGDGRCVTCIYYLNKDWDAKVSGGILRIFPEGKAQFADIEPKFDRLLFFWSDRRNPHEVQPAYATRYAITVWYFDADERARAKVKYLTGEKGVRVELNKPSDSVGKDVF

SEQ ID NO: 28 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 29 - protein, 1366 aa, Homo sapiens; identical to SEQ ID NO: 24

SEQ ID NO: 30 - protein, 6 aa, synthetic construct
  REGION 1..6: note = loop of CPP
RGFRRR

(19) United States
(12) Patent Application Publication
(10) Pub. No.: US 2025/0171821 A1
(43) Pub. Date: May 29, 2025

(54) HIF-1 ACTIVATORS AND USES THEREOF

(71) Applicant: Ramot at Tel-Aviv University Ltd., Tel-Aviv (IL)

(72) Inventors: Maayan GAL, Tel Aviv (IL); Evgeny WEINBERG, Tel Aviv (IL); Lihi ADLER-ABRAMOVICH, Tel Aviv (IL); Ran TOHAR, Tel Aviv (IL)

(73) Assignee: Ramot at Tel-Aviv University Ltd., Tel-Aviv (IL)

(21) Appl. No.: 19/049,063

(22) Filed: Feb. 10, 2025

Related U.S. Application Data
(63) Continuation of application No. PCT/IL2023/050839, filed on Aug. 10, 2023.
(60) Provisional application No. 63/396,661, filed on Aug. 10, 2022.

(51) Int. Cl. C12P 21/00; A61K 31/16; A61K 31/194; A61K 31/437; A61K 31/4418; A61K 31/472; A61K 31/515; A61K 31/53; A61K 31/5377; A61K 38/17; C12N 5/077; C12N 15/113

(52) U.S. Cl. CPC C12P 21/00; A61K 31/16; A61K 31/194; A61K 31/437; A61K 31/4418; A61K 31/472; A61K 31/515; A61K 31/53; A61K 31/5377; A61K 38/1709; C12N 5/0654; C12N 5/0656; C12N 15/113; C12N 2310/14; C12N 2501/60; C12N 2501/999

(57) ABSTRACT

Method of increasing collagen production and tissue regeneration or repair using an agent that increases an amount of Hypoxia-inducible factor-1 (HIF-1) in the cells, the amount being selected to cause an increase in the amount of collagen production in the cells are disclosed. Also disclosed non-human cell cultures which comprise an activator of HIF-1.

### RELATED APPLICATIONS

This application is a Continuation of PCT Patent Application No. PCT/IL2023/050839 having International filing date of Aug. 10, 2023, which claims the benefit of priority under 35 USC § 119(e) of U.S. Provisional Patent Application No. 63/396,661 filed on Aug. 10, 2022. The contents of the above applications are all incorporated by reference as if fully set forth herein in their entirety.

### SEQUENCE LISTING STATEMENT

The XML file, entitled 102631SequenceListing.xml, created on Jan. 20, 2025, comprising 82,863 bytes, submitted concurrently with the filing of this application is incorporated herein by reference.

### FIELD AND BACKGROUND OF THE INVENTION

The present invention, in some embodiments thereof, relates to activators of Hypoxia-inducible factor-1 (HIF-1) for increasing cellular and/or extracellular collagen.

Hypoxia-inducible factor-1 (HIF-1) is a key mediator of oxygen homeostasis that was first identified as a transcription factor that is induced and activated by decreased cellular oxygen levels. Upon activation, HIF-1 upregulates the transcription of genes that promote adaptation and survival under hypoxic conditions. HIF-1 is a heterodimer composed of an oxygen-regulated subunit known as HIF-1α and a constitutively expressed HIF-1β subunit. In general, the availability and activity of the HIF-1α subunit determines the activity of HIF-1. Subsequent studies have revealed that HIF-1 is also activated by environmental and physiological stimuli that range from iron chelators to hormones.

Under normal oxygen levels (i.e., normoxia), HIF-1 activity is heavily regulated by the family of HIF-prolyl hydroxylases (PHDs). The latter hydroxylates HIF on specific prolines along its primary sequence. This essential interaction tags HIF as a substrate for the von Hippel-Lindau (VHL) E3-ligase complex that ubiquitinates HIF for degradation in the proteasome. Hydroxylation by PHD is oxygen- and Iron (Fe)-dependent and under hypoxia, reduced oxygen levels hamper the ability of PHD to hydroxylate HIF. Given that the non-hydroxylated HIF is not marked for degradation in the proteasome, it is fast accumulating, initiating an accelerated transcription of genes under the control of hypoxia-responsive elements (HRE) promoter.

Over activation of HIF-1α through inhibition of PHD2 has been proposed as a potential therapeutic target for various hypoxia-related diseases including anemia, myocardial infarction, stroke, peripheral arterial disease (Kwon et al., Bioorganic & Medicinal Chemistry Letters 21 (2011) 4325-4328; US Patent Application No. 20140364419).

Hypoxia and the composition of the extracellular matrix (ECM) are directly related and a direct relation between PHD activity and levels of collagen exists (Strowitzki M J et al., Pharmacol Res. 2019; 147: 104364 and Markway B D, et al., Arthritis Res Ther. 2013; 15: R92).

PHD2 deficiency and a HIF-PHD inhibitor, DMOG, has been correlated with increased levels of collagen in conditions of ischemic anastomoses (Strowitzki M J, JCI Insight. 2021; 6. doi: 10.1172/jci.insight.139191). On the other hand, mice treated with DMOG showed a significant decrease in collagen deposition after dextran sulfate sodium-induced intestinal fibrosis (M. C. Manresa, et al., Am. J. Physiol.—Gastrointest. Liver Physiol. 311 (2016) G1076-G1090).

Among the various tissues in which HIF plays an imperative regulatory role are the gingiva and periodontal ligament. It was recently shown that accumulation of collagen type I is upregulated in hypoxia in these tissues (Morimoto et al., J Periodontol. 2021; 92: 1635-1645).

The N-terminal transactivation domain (NTAD) of the HIF-1 protein is responsible for initiating the transcriptional activation of target genes in response to hypoxia (Cheng-Jun Hu et al., 2007; Molecular Biology of the Cell Vol. 18, 4528-4542).

Nwogu J I., et al., 2001 (Circulation 104:2216-2221) describe a decrease in Collagen type I and III and improvement of left ventricular function after myocardial infraction following treatment of rats with FG041, an inhibitor of prolyl 4 hydroxylase (P4H).

Additional background art includes US Patent Application No. 20210353612, 20220143211 and 20210162008 which teach tissue regeneration (including skin, bone and cartilage) and wound healing by using HIF activators.

### SUMMARY OF THE INVENTION

According to an aspect of some embodiments of the invention there is provided an ex vivo method of increasing collagen production in cells comprising contacting the cells with an agent that increases an amount of Hypoxia-inducible factor-1 (HIF-1) in the cells, the amount being selected to cause an increase in the amount of collagen production in the cells.

According to an aspect of some embodiments of the invention there is provided cell culture comprising non-human cells and a culture medium which comprises an activator of Hypoxia-inducible factor-1 (HIF-1).

According to an aspect of some embodiments of the invention there is provided a method of inducing tissue regeneration or cell repair in a subject in need thereof comprising administering to the subject an HIF activator in an amount which brings about an up-regulation of collagen, thereby inducing tissue regeneration or cell repair.

According to an aspect of some embodiments of the invention there is provided method of generating collagen comprising:
(a) increasing collagen production according to the method of some embodiments of the invention; and
(b) isolating the collagen from the cells or from the medium.

According to an aspect of some embodiments of the invention there is provided a method of generating a food product comprising:
(a) increasing collagen production according to the method of some embodiments of the invention;
(b) isolating the cells from the medium;
(c) combining the cells or the collagen isolated from the cells with a food or feed product.

According to an aspect of some embodiments of the invention there is provided a food product generated according to the method of some embodiments of the invention.

According to some embodiments of the invention, the method is a non-invasive method.

According to some embodiments of the invention, the agent that increases an amount of Hypoxia-inducible factor-1 (HIF-1) in the cells is a HIF-1 activator.

According to some embodiments of the invention, the contacting comprises culturing the cells in a medium comprising an activator of HIF-1.

According to some embodiments of the invention, the cells comprise non-human cells.

According to some embodiments of the invention, the activator of the HIF-1 is provided in an amount which brings about an up-regulation of collagen.

According to some embodiments of the invention, the activator prevents binding between HIF-prolyl hydroxylase (PHD) and the HIF-1.

According to some embodiments of the invention, the activator reduces hydroxylation under normoxia of the HIF-1 by the HIF-PHD.

According to some embodiments of the invention, the activator comprises a peptide agent.

According to some embodiments of the invention, the peptide comprises at least 15 and no more than 25 consecutive amino acid residues of the HIF-1α polypeptide set forth by SEQ ID NO: 5.

According to some embodiments of the invention, the peptide comprises at least 15 and no more than 25 consecutive amino acid residues of the EPAS1 (HIF-2α) polypeptide set forth by SEQ ID NO: 6.

According to some embodiments of the invention, the peptide is set forth by SEQ ID NO: 4.

According to some embodiments of the invention, the peptide is set forth by SEQ ID NO: 2.

According to some embodiments of the invention, the peptide further comprises a cell penetrating peptide.

According to some embodiments of the invention, the cell penetrating peptide is set forth by SEQ ID NO: 1.

According to some embodiments of the invention, the activator is a small molecule agent.

According to some embodiments of the invention, the small molecule agent is ML228 or a derivative thereof.

According to some embodiments of the invention, the activator of the HIF-1 is an iron chelator.

According to some embodiments of the invention, the iron chelator is Deferoxamine mesylate (DFO).

According to some embodiments of the invention, the activator of the HIF-1 comprises an analogue of 2-oxoglutarate (2-OG).

According to some embodiments of the invention, the small molecule is selected from the group consisting of Roxadustat (FG-4592), Daprodustat, Molidustat, Enarodustat (JTZ-951) and Vadadustat, or derivatives thereof.

According to some embodiments of the invention, the activator of the HIF-1 is provided at a concentration which does not induce cell death.

According to some embodiments of the invention, the HIF-1 activator is present in the medium at a concentration between 1 nanomolar (1 nM)-100 micromolar (100 μM).

According to some embodiments of the invention, a concentration of the HIF-1 activator in the medium is between 1 nM-10 μM.

According to some embodiments of the invention, the contacting comprises genetically modifying the cells so that they express exogenous HIF-1.

According to some embodiments of the invention, the activator of the HIF-1 comprises an agent which downregulates expression of HIF-PHD.

According to some embodiments of the invention, the agent is an RNAi molecule.

According to some embodiments of the invention, the cells are of an edible animal.

According to some embodiments of the invention, the non-human cells are cells of an edible animal.

According to some embodiments of the invention, the non-human cells comprise non-human connective tissue cells.

According to some embodiments of the invention, the cells are genetically modified to express collagen.

According to some embodiments of the invention, the cells comprise non-human stem cells or progenitor cells.

According to some embodiments of the invention, the cells further comprise non-human muscle cells.

According to some embodiments of the invention, the cells are selected from the group consisting of fibroblasts, osteoblasts and adipocytes.

According to some embodiments of the invention, the non-human cells are selected from the group consisting of fibroblasts, osteoblasts and adipocytes.

According to some embodiments of the invention, the culturing is effected in the absence of serum.

According to some embodiments of the invention, the culturing is effected in the presence of an osteogenic differentiation medium.

According to some embodiments of the invention, the collagen comprises type I collagen.

According to some embodiments of the invention, the non-human cells comprise at least 10% more collagen compared to the non-human cells cultured in the culture medium in an absence of the activator of the HIF-1.

According to some embodiments of the invention, the contacting the cells with the agent is effected under hypoxia.

According to some embodiments of the invention, the non-human cells are under hypoxia.

According to some embodiments of the invention, the cells comprise human cells.

According to some embodiments of the invention, the cells are human cells.

According to some embodiments of the invention, the method further comprises isolating the collagen from the cells.

According to some embodiments of the invention, the isolating of the collagen is from the cells.

According to some embodiments of the invention, the method further comprising combining the cells or the collagen isolated from the cells with a taste modifying agent.

According to some embodiments of the invention, the HIF activator is formulated as a cosmetic.

According to some embodiments of the invention, the tissue regeneration is at a site selected from the group consisting of bone, skin, hair and cartilage.

According to some embodiments of the invention, the tissue regeneration is of the skin.

According to some embodiments of the invention, the administering comprises topically administering.

According to some embodiments of the invention, the method further comprises administering to the subject at least one agent selected from the group consisting of hyaluronic acid (HA) and Botulinum Toxin-Type A (Botox).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 depicts the chemical structure of ML228.

FIGS. 2A-D. Effect of ML228 on Collagen-I levels in fibroblasts (FIGS. 2A-B) and osteoblasts (FIGS. 2C-D). Cells were cultured for 48 hours with variable concentrations of ML228 and stained with Collagen-I antibody (green) and DAPI (blue). Images were taken by confocal microscopy and analyzed with imageJ to evaluate fluorescence intensity. FIGS. 2A-B: Collagen-I immunofluorescence in fibroblast cells. FIG. 2A—a bar chart showing absolute mean fluorescence intensity (FLI) of the fibroblast cells as a function of ML228 concentrations (total FLI, without normalization to number of cells). FIG. 2B—representative confocal images of Collagen-I immunofluorescence in fibroblast cells following treatment with increasing concentrations of ML228 (0.32, 0.62 and 5 μM ML228) or in non-treated cells. FIGS. 2C-D: Collagen-I immunofluorescence in osteoblast cells. FIG. 2C—a bar chart showing absolute mean fluorescence intensity of the osteoblast cells as a function of ML228 concentrations (total FLI, without normalization to number of cells). FIG. 2D—representative confocal images of Collagen-I immunofluorescence in osteoblast cells following treatment with increasing concentrations of ML228 (0.32, 0.62 and 5 μM ML228) or in non-treated cells.

FIGS. 3A-F. Cellular viability and toxicity. FIG. 3A—Fibroblasts and osteoblasts were incubated with variable concentrations of ML228 for 48 hours and cellular viability was evaluated via Cell-Titer assay measuring cellular ATP levels, an indicator of metabolically active cells. The curve shows the percentage of live cells relative to non-treated cells. FIGS. 3B-C—Representative confocal images and fluorescence intensity of live (green) and dead (red) fibroblast cells (FIG. 3B) and osteoblast cells (FIG. 3C). FIGS. 3D-E—quantification of the live/dead assay. Intensity is shown relative to non-treated cells. Shown are positive cells relative to non-treated cells in various ML228 concentrations for fibroblast cells (FIG. 3D) and osteoblast cells (FIG. 3E). FIG. 3F—fluorescence intensity of green and red color is shown relative to nontreated cells. The results show that an increase in ML228 concentrations results in a decrease in metabolically active cell viability as measured by ATP levels but not an increase in cell death.

FIG. 4 is a Western blot analysis showing protein levels of HIF-1α, Collagen-I and β-tubulin as a function of variable ML228 concentration. Fibroblast cells were incubated with ML228 for 48 hours and cellular lysate was analyzed by Western blot.

FIGS. 5A-C depict the effect of ML228 on Collagen-I levels in human gingival fibroblasts cells (HGF). Cells were cultured for 48 hours with variable concentrations of ML228 and then stained for Collagen-I (green color), β-tubulin (red color), and DNA (DAPI, blue color). FIG. 5A—Representative images of HGF cells in the presence of various concentrations of ML228: 312 nM, 625 nM and 5000 nM. “DMSO” (dimethyl sulfoxide) which was used to dissolve ML228 was used as a negative control in the absence of ML228 (upper panels). The final DMSO concentration in the cells was 0.04%. The right panels show “merged” images of the DAPI, β-tubulin and Collagen-I stainings. Scale bar: 100 μm. FIG. 5B—level of Collagen I normalized against housekeeping protein β-tubulin. FIG. 5C—Mean fluorescence intensity relative to nontreated cells (DMSO) indicating levels of Collagen-I normalized to the number of cells as counted by the nuclear DAPI staining (FIG. 5C).

FIG. 6 depicts Western blot analysis showing HIF-1α, Collagen-I, and β-tubulin protein levels as a function of variable ML228 concentrations. Fibroblast cells were incubated with variable concentrations of ML228 for 48 hours, and the cellular lysate was analyzed.

FIG. 7 depicts schematic illustration of a proposed mechanism of action of a HIF1-α activator (ML228) or a HIF-prolyl hydroxylase (PHD) inhibitor. Under normoxia and without treatment with either ML228 or a HIF-PHD inhibitor, the activity of hydroxylated HIF is regulated via its degradation. The application of ML228 or a HIF-PHD inhibitor leads to higher HIF activity and accumulation of Collagen-I.

FIGS. 8A-C depict the effect of ML228 on Collagen-I levels in HGF cells. Cells were cultured for 48 hours with variable concentrations of ML228 and stained against Collagen-I, β-tubulin and DAPI (green, red and blue, respectively). FIGS. 8A and 8B—Representative images of HGF cells treated with ML228. Scale bar: 100 μm. FIG. 8C—Mean normalized fluorescence intensity of Collagen-I relative to non-treated cells normalized to number of cells by DAPI staining. Images were taken by confocal microscopy and analyzed using Fiji 2 to evaluate fluorescence intensity. Values were measured using Color Threshold tab and normalized to the mean collagen intensity in the non-treated cells. It is noted that the symbol “uM” as used herein and/or in any Figure or description thereof refers to micromolar (μm); Scale bar: 200 μm.

FIGS. 9A-C depict the effect of ML228 on Collagen-I levels in MG-63 osteoblasts. Cells were cultured for 48 hours with variable concentrations of ML228, and stained with Collagen-I antibody (green), β-tubulin (red) and DAPI (blue). FIG. 9A—Representative confocal images of osteoblasts following treatment with ML228. Scale bar: 100 μm. FIG. 9B—Mean fluorescence intensity relative to non-treated cells and normalized relative to the number of cells, as a function of ML228 concentration. FIG. 9C—Cellular viability assay of osteoblast cells treated with ML228. Cells were incubated with variable concentrations of ML228 for 48 hours, and cellular viability was evaluated via the live/dead assay. Representative confocal images (top) of live (green) and dead (red) cells treated with various concentrations of ML228, and with 70% ethanol as a reference for dead cells (right column).

FIG. 10 shows that ML228 does not have autofluorescence. Immunofluorescence of non-treated (DMSO) and treated HGF cells with 5 μM of ML228 without DAPI, β-tubulin and Collagen-I antibodies. Scale bar: 100 μm.

FIG. 11 depicts binocular and fluorescence micrographs of MG63 cells mineralized matrix stained with Alizarin red. MG-63 cells were cultured under mineralizing conditions; and changing media every 2-3 days for total 7 days of treatment in the presence or absence Def16-EPAS1 (SEQ ID NO: 3) at the indicated concentrations. Typical mineralized nodules were observed in all analyzed conditions except for in the non-treated (NT) cells which included the osteogenic differentiation medium, yet without the Def16-EPAS1 peptide. Binoculars 4× lenses used, confocal 10× lenses. Scale bar: 500 μm. Red color—shows Alizarin Red staining in 647 nm. Bright field—white color.

FIG. 12 depicts Picrosirius red staining for detection of collagen type I (red) and collagen type III (green) in MG-63 cells. MG-63 cells were incubated with the Def16-EPAS1 peptide for 7 days. Scale bar: 200 μm. BF refers to Bright field. It is noted that enhanced levels of collagen types I and III are observed following treatment with the DEF16-EPAS1 peptide.

FIG. 13 depicts fluorescence polarization (FP) showing direct binding of FITC-labeled peptides EPAS1 (blue circles) and HIF (green triangles) to PHD enzyme. Variable concentrations of each selected peptide were incubated with PHD bound to the FITC-labeled peptide. Polarization levels were read and normalized values were fitted to a single site model. The graph shows the bound fraction of PHD enzyme to each peptide.

FIGS. 14A-B depict the effect of Deferoxamine mesylate (DFO), an iron chelator, on Collagen-I levels in MG-63 cells. Cells were cultured for 48 hours with variable concentrations of DFO (0.6 μM, 5 μM or 20 μM) and then stained for Collagen-I (green color), β-tubulin (red color), and DNA (DAPI staining; blue color). FIG. 14A—representative immunofluorescence images of MG-63 cells cultured with 0.6 μM DFO (second panels from the top), 5 μM DFO (third panels from the top), or 20 μM DFO (forth panels from the top), or remained untreated (DMSO, negative control; first panels from the top). Scale bar: 100 μm. FIG. 14B is a bars plot showing the mean normalized fluorescence intensity of Collagen-I relative to non-treated cells, and normalized to tubulin levels.

FIG. 15 depicts Alizarin red S staining and Picrosirius Red staining of MG-63 after incubation with osteogenic factors and the Def16-EPAS1 peptide for indicated time points. Cells were supplemented with osteogenic factors: ascorbic acid (50 μg/mL) and β-glycerophosphate (10 mM) together with 10 μM of Def16-EPAS1 peptide for the indicated time periods. Medium was changed every 2 days. 7, 14, 21 and 28 days after induction of differentiation, the production of extracellular calcified mineral matrix was visualized by Alizarin Red staining. Picrosirius Red staining depicts the presence of Collagen-I.

FIGS. 16A-B depict the effect of the Def16-EPAS1 peptide on Collagen-I production in bovine dermal fibroblasts. FIG. 16A shows representative confocal images that visualize DNA, β-tubulin, and Collagen-I in Bovine Dermal fibroblast cells. FIG. 16B shows the corresponding box plot of the mean fluorescence intensity (FLI) of total Collagen-I normalized to β-tubulin as a function of Def16-EPAS1 concentration.

FIGS. 17A-E depict the effect of ML228 on NIH-3T3 cells. FIG. 17A—Chemical structure of ML228 also known as N-([1,1′-Biphenyl]-4-ylmethyl)-6-phenyl-3-(2-pyridinyl)-1,2,4-triazin-5-amine. Cells were cultured for 48 hours with variable concentrations of ML228 and then labelled for collagen type 1. FIGS. 17B-C—Immunofluorescence measurements for Phase Object Confluence (%) (FIG. 17B) and Total Collagen type-1 Integrated Intensity Per Image normalized to Phase Area Confluence (GCU×μm²/Image/%) (FIG. 17C). FIG. 17D—NIH-3T3 Viability test using Alamar blue. Error bars indicate SEM (Standard Error of the Mean). Asterisks show significance following ordinary one-way ANOVA. FIG. 17E—Representative confocal images of NIH-3T3 treated with 5 μM ML228 (left panels), 0.63 μM ML228 (middle panels) and control with DMSO (right panels). Upper panels: Immunostaining for Collagen type-1 (“Coll1”; green). Lower panels: merged with staining for F-Actin (red), and nuclear staining using DAPI (blue). Scale bars, 20 μm.

FIGS. 18A-D depicts the effect of Daprodustat on NIH-3T3 cells. FIG. 18A—Chemical structure of Daprodustat also known as GSK1278863 or N-[(1,2-Dicyclohexylhexahydro-2,4,6-trioxo-5-pyrimidinyl)carbonyl]glycine. Cells were cultured for 48 hours with variable concentrations of Daprodustat and then labelled for collagen type 1. FIGS. 18B-C—Immunofluorescence measurements for number of cells (per image) (FIG. 18B) and total Collagen-I Integrated Intensity per Image normalized to number of cells (GCU×μm²/Image/1/image) (FIG. 18C). Error bars indicate SEM. Asterisks show significance following ordinary one-way ANOVA. FIG. 18D—Wide-field images of NIH-3T3 treated with 5 μM Daprodustat (left panels), 1.25 μM Daprodustat (middle panels) and control with DMSO (right panels). Upper panels: Immunostaining for Collagen type-1 (green). Lower panels: merged images of Collagen type-1 with nuclear staining using DRAQ5 (far-red). Scale bars, 50 μm.

FIGS. 19A-D depict the effect of Enarodustat on NIH-3T3 cells. FIG. 19A—Chemical structure of Enarodustat, also known as JTZ 951 or N-[[7-Hydroxy-5-(2-phenylethyl)[1,2,4]triazolo[1,5-a]pyridin-8-yl]carbonyl]glycine. Cells were cultured for 48 hours with variable concentrations of Enarodustat and then labelled for collagen type 1. FIGS. 19B-C—Immunofluorescence measurements for number of cells (per image) (FIG. 19B) and total Collagen-I Integrated Intensity per Image normalized to number of cells (GCU×μm²/Image/1/image) (FIG. 19C). Error bars indicate SEM. Asterisks show significance following ordinary one-way ANOVA. FIG. 19D—Wide-field images of NIH-3T3 treated with 5 μM Enarodustat (left panels), 0.31 μM Enarodustat (middle panels) and control with DMSO (right panels). Upper panel: Immunostaining for Collagen type-1 (“Coll1”; green). Lower panels: merged images of Collagen type-1 and nuclear staining using DRAQ5 (far-red). Scale bars, 50 μm.

FIGS. 20A-D depict the effect of Molidustat on NIH-3T3 cells. FIG. 20A—Chemical structure Molidustat, known also as BAY 85-3934 or 1,2-Dihydro-2-[6-(4-morpholinyl)-4-pyrimidinyl]-4-(1H-1,2,3-triazol-1-yl)-3H-pyrazol-3-one. Cells were cultured for 48 hours with variable concentrations of Molidustat and then labelled for collagen type 1. FIGS. 20B-C—Immunofluorescence measurements for number of cells (per image) (FIG. 20B) and total Collagen-I Integrated Intensity per Image normalized to number of cells (GCU×μm²/Image/1/image) (FIG. 20C). Error bars indicate SEM. Asterisks show significance following ordinary one-way ANOVA. FIG. 20D—Wide-field images of NIH-3T3 treated with 5 μM Molidustat (left panels), 0.31 μM Molidustat (middle panels) and control with DMSO (right panels). Upper panels: Immunostaining for Collagen type-1 (“Coll1”; green). Lower panel: merged images of Collagen type-1 with nuclear staining using DRAQ5 (far-red). Scale bars, 50 μm.

FIGS. 21A-D depict the effect of Vadadustat on NIH-3T3 cells. FIG. 21A—Chemical structure Vadadustat, also known as AKB 6548, PG 1016548 or N-[[5-(3-Chlorophenyl)-3-hydroxy-2-pyridinyl]carbonyl]glycine. Cells were cultured for 48 hours with variable concentrations of Vadadustat and then labelled for Collagen type 1. FIGS. 21B-C—Immunofluorescence measurements for number of cells (per image) (FIG. 21B) and Total Collagen-I Integrated Intensity per Image normalized to number of cells (GCU×μm²/Image/1/image) (FIG. 21C). Error bars indicate SEM. Asterisks show significance following ordinary one-way ANOVA. FIG. 21D—Wide-field images of NIH-3T3 treated with 5 μM Vadadustat (left panels), 0.63 μM Vadadustat (middle panels) and control with DMSO (right panels). Upper panels: Immunostaining for Collagen type-1 (“Coll1”; green). Lower panel: merged images of Collagen type-1 with nuclear staining using DRAQ5 (far-red). Scale bars, 50 μm.

FIGS. 22A-D depict the effect of Roxadustat on NIH-3T3 cells. FIG. 22A—Chemical structure of Roxadustat, also known as FG 4592, ASP 1517 or N-[(4-Hydroxy-1-methyl-7-phenoxy-3-isoquinolinyl)carbonyl]glycine. Cells were cultured for 48 hours with variable concentrations of Roxadustat and then labelled for Collagen type 1. FIGS. 22B-C—Immunofluorescence measurements for number of cells (per image) (FIG. 22B) and Total Collagen-I Integrated Intensity per Image normalized to number of cells (GCU×μm²/Image/1/image) (FIG. 22C). Error bars indicate SEM. Asterisks show significance following ordinary one-way ANOVA. FIG. 22D—Wide-field images of NIH-3T3 treated with 5 μM Roxadustat (left panels), 0.63 μM Roxadustat (middle panels) and control with DMSO (right panels). Upper panels: Immunostaining for Collagen type-1 (“Coll1”; green). Lower panel: merged images of Collagen type-1 with nuclear staining using DRAQ5 (far-red). Scale bars, 50 μm.

### DESCRIPTION OF SPECIFIC EMBODIMENTS OF THE INVENTION

The present invention, in some embodiments thereof, relates to activators of Hypoxia-inducible factor-1 (HIF-1) for increasing cellular and/or extracellular collagen.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Collagen is the most abundant scaffold protein in tissues. For example, Collagen provides the structural framework for bone formation and plays a vital role in its strength and flexibility in calcification of the matrix. Given its essential role in an array of cellular processes, collagen is used in a broad range of applications in the pharmaceutical and food industries. Although many advantages are associated with natural collagen derived from mammalian cells, high yield expression of collagen is challenging and not cost-effective, and thus the main source of external collagen is from animal tissues.

Hypoxia-inducible factor (HIF-1) is an oxygen-dependent transcriptional activator, which plays crucial roles in the angiogenesis of tumors and mammalian development. HIF-1 consists of a constitutively expressed HIF-1β subunit and one of three subunits (HIF-1α, HIF-2α or HIF-3α). The stability and activity of HIF-1α are regulated by various post-translational modifications, hydroxylation, acetylation, and phosphorylation. Under normoxia, the HIF-1α subunit is rapidly degraded via the von Hippel-Lindau tumor suppressor gene product (vHL)-mediated ubiquitin-proteasome pathway. The association of vHL and HIF-1α under normoxic conditions is triggered by the hydroxylation of prolines and the acetylation of lysine within a polypeptide segment known as the oxygen-dependent degradation (ODD) domain. During hypoxic conditions HIF-1α subunit becomes stable and interacts with coactivators such as p300/CBP to modulate its transcriptional activity.

HIF-1 acts as a master regulator of numerous hypoxia-inducible genes under hypoxic conditions. The heterodimer HIF-1 binds to the hypoxic response elements (HREs) of target gene regulatory sequences, resulting in the transcription of genes implicated in the control of cell proliferation/survival, glucose/iron metabolism and angiogenesis, as well as apoptosis and cellular stress. Some of these direct target genes include glucose transporters, the glycolytic enzymes, erythropoietin, and angiogenic factor vascular endothelial growth factor (VEGF).

The present inventors have surprisingly uncovered that enhanced collagen accumulation can be imparted via activation of the hypoxia pathway using a HIF activator, such as a small molecule HIF-1 activator (e.g., ML228), an activator of HIF-1 which prevents binding between HIF-prolyl hydroxylase (PHD) and HIF-1 such as a peptide agent, an activator of HIF-1 which reduces hydroxylation of HIF-1 by the HIF-PHD enzyme such as an iron chelator or an analogue (e.g., a competitive inhibitor) of 2-oxoglutarate (2-OG). Thus, the present inventors have uncovered that HIF-1 activators can increase the amount and/or activity of HIF-1 to a level which is sufficient for increasing production and accumulation of collagen by the cells.

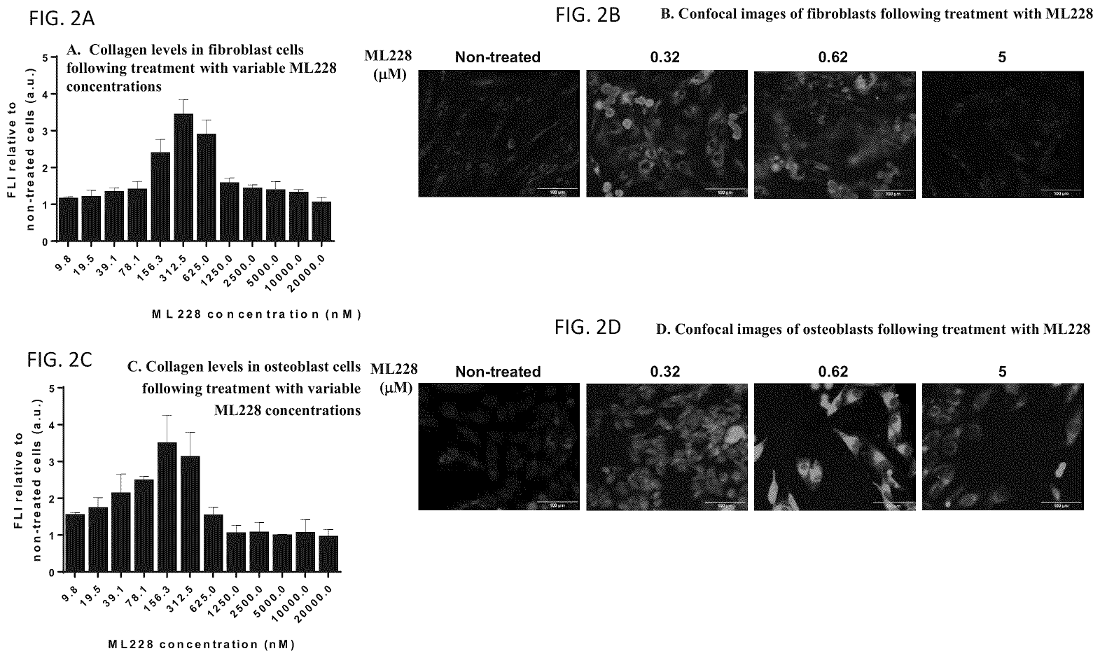

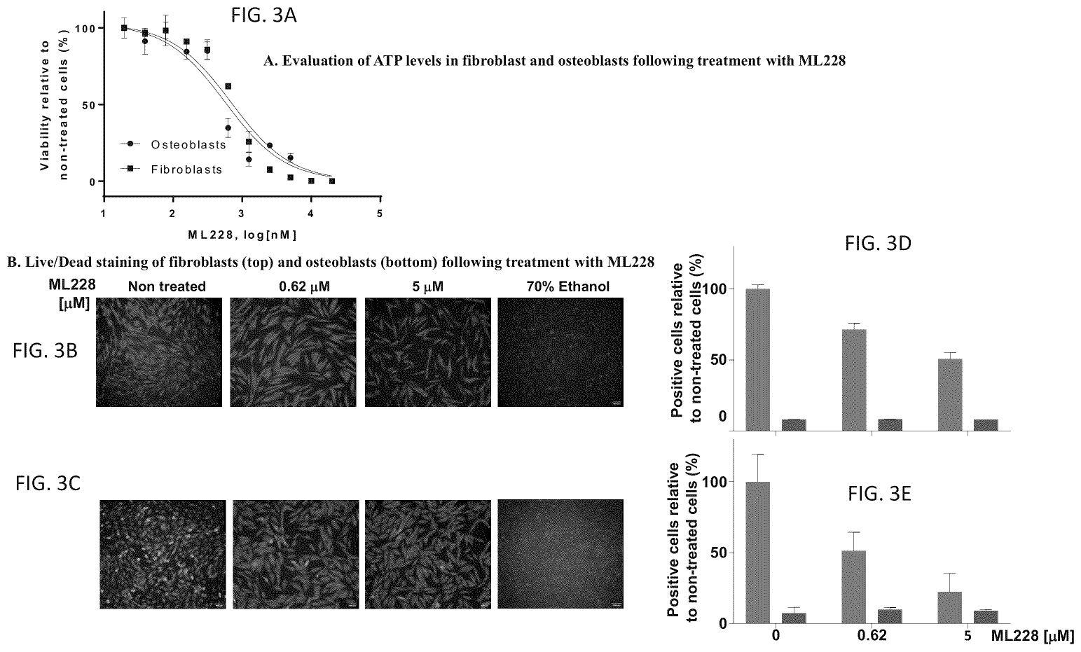

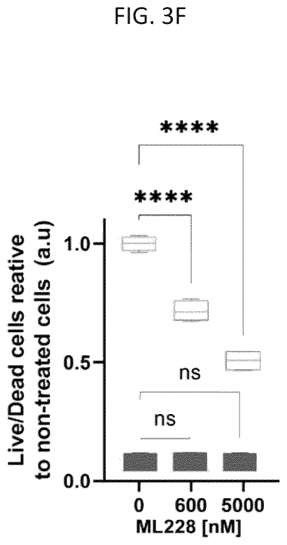

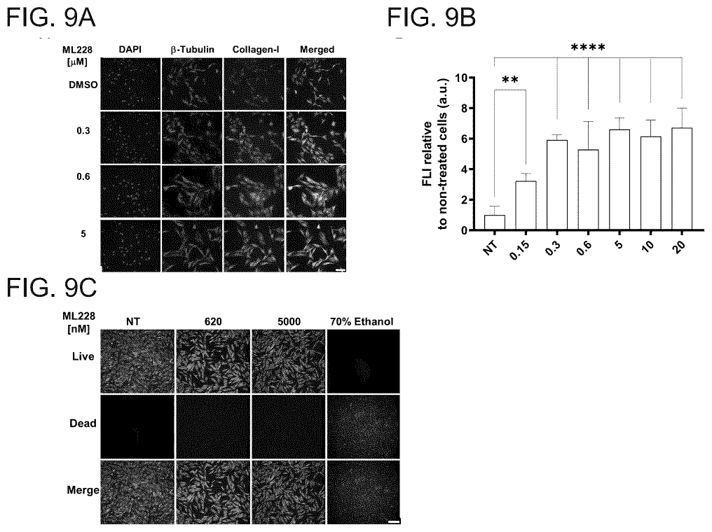

Indeed, Examples 1 and 2 of the Examples section which follows show that more than a three-fold increase in the levels of collagen was obtained when fibroblast and osteoblast cells were incubated with variable concentrations of ML228 (FIGS. 2A-D; FIGS. 9A-C), yet without imparting cellular toxicity (FIGS. 3B-F).

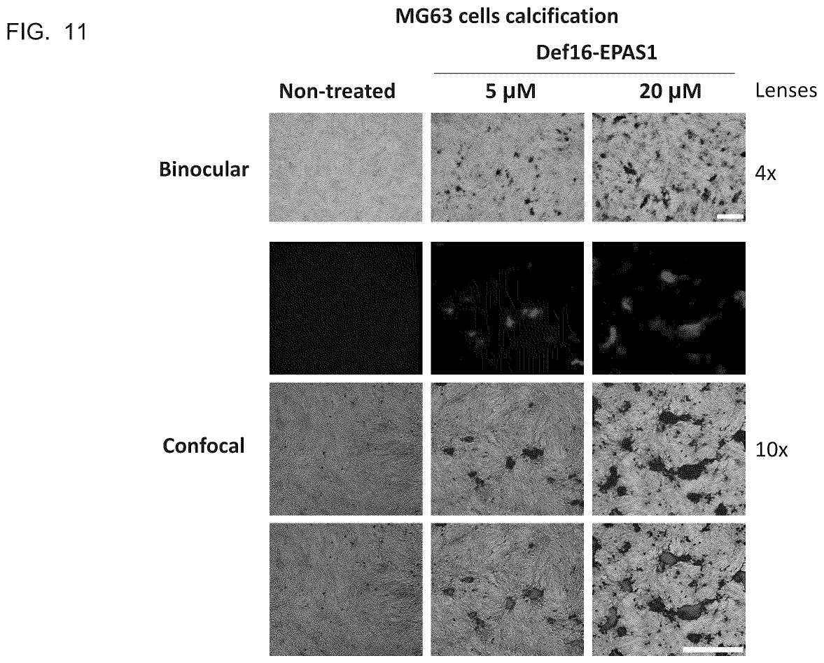

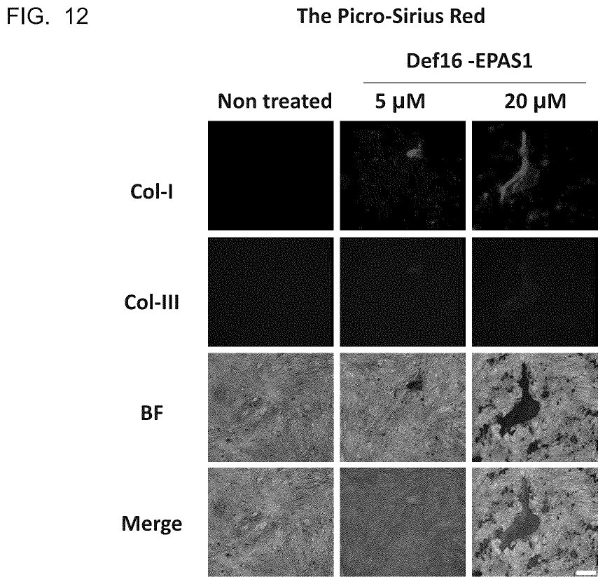

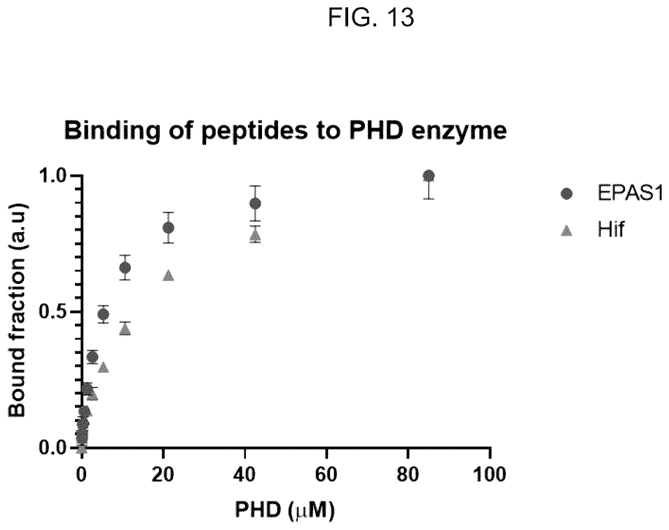

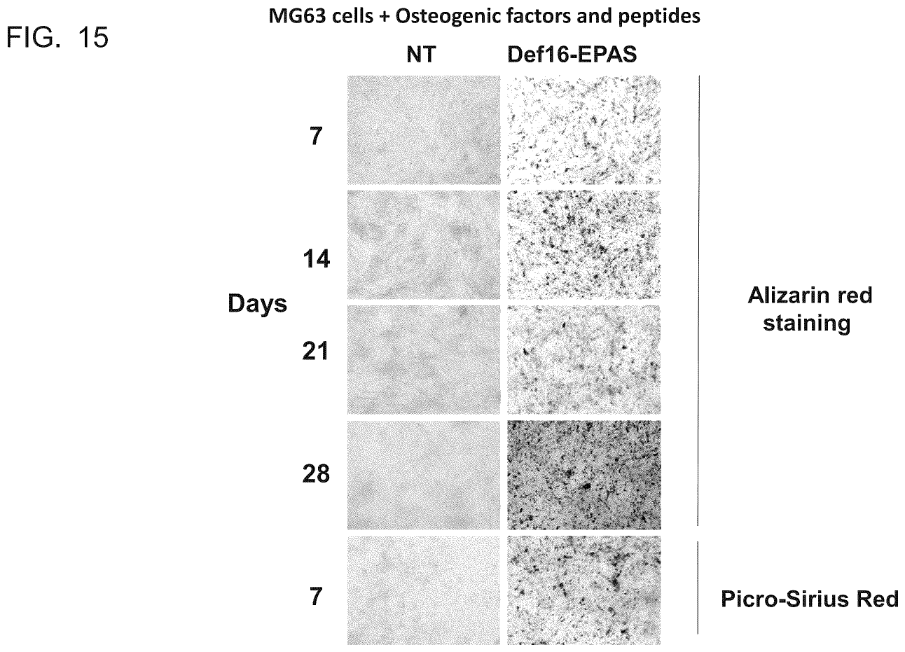

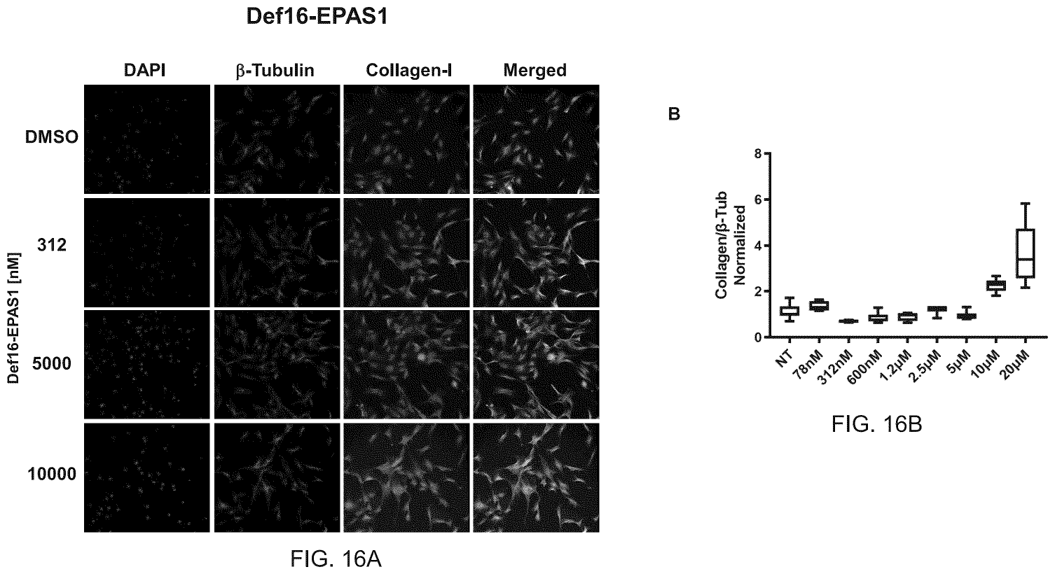

In addition, Example 3 of the Examples section which follows shows that an EPAS1-derived peptide, which specifically binds to a HIF-PHD polypeptide (FIG. 13), is capable of increasing over-production of Collagen-I and III (FIGS. 12 and 16A-B), induction of deposition of calcium (FIG. 11) and formation of a mineralizing matrix in MG-63 cells (FIG. 15). These results demonstrate that the HIF mimicking peptides increase activation of HIF to a level which stimulated Collagen accumulation.

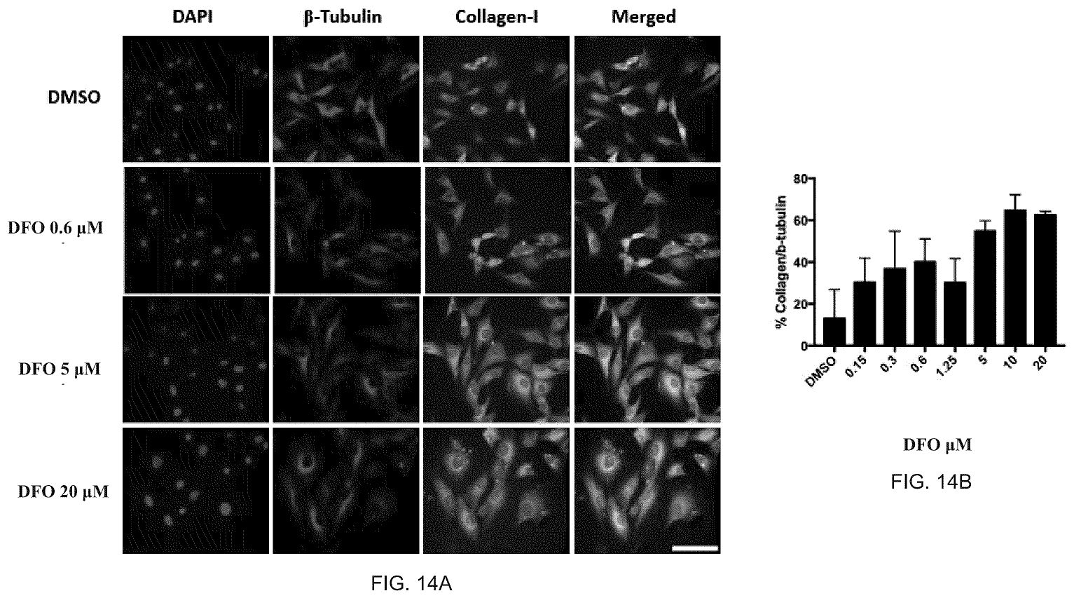

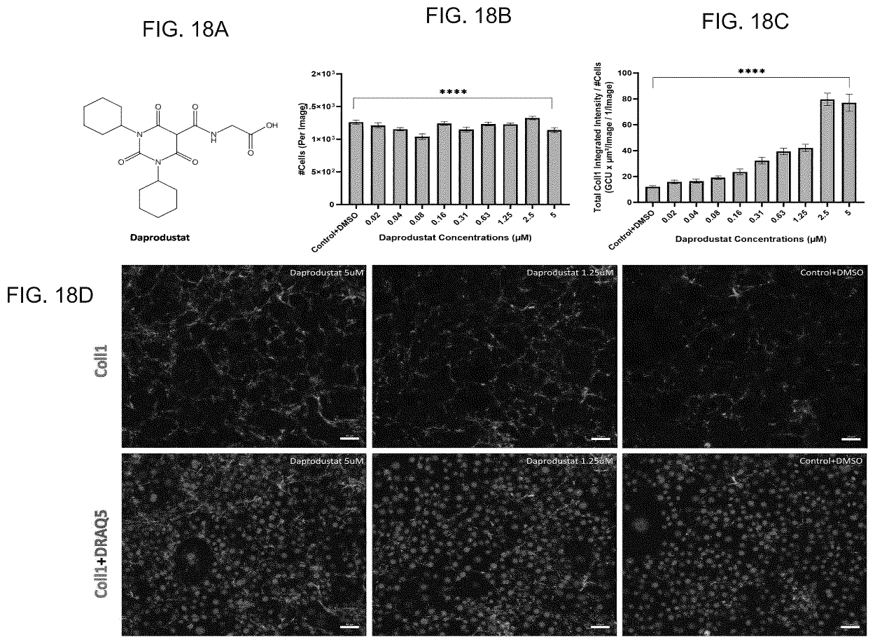

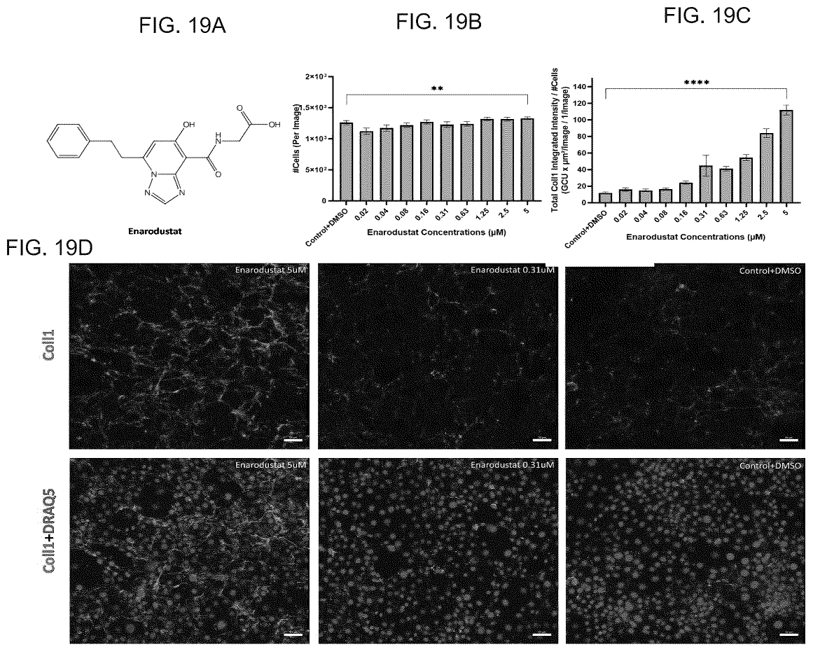

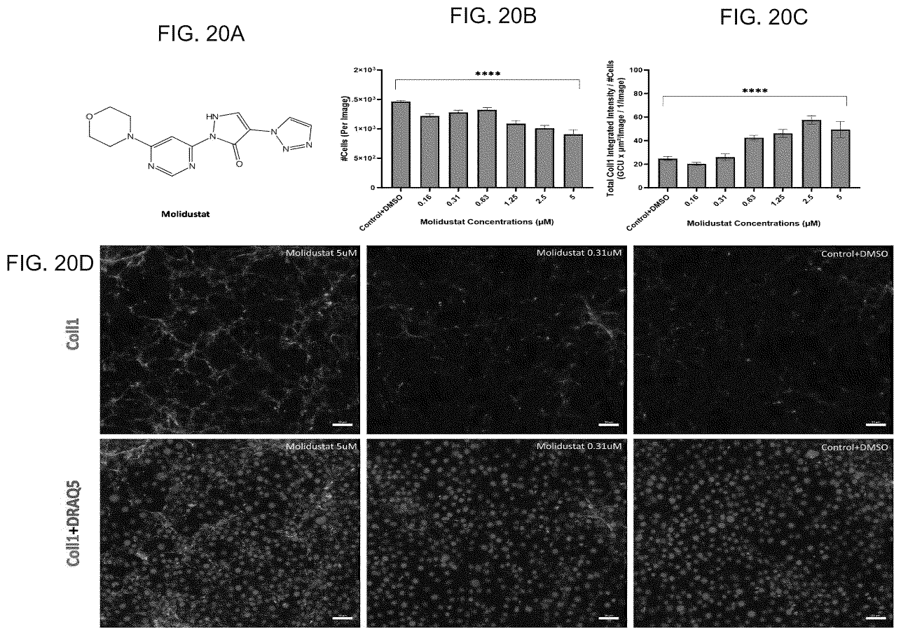

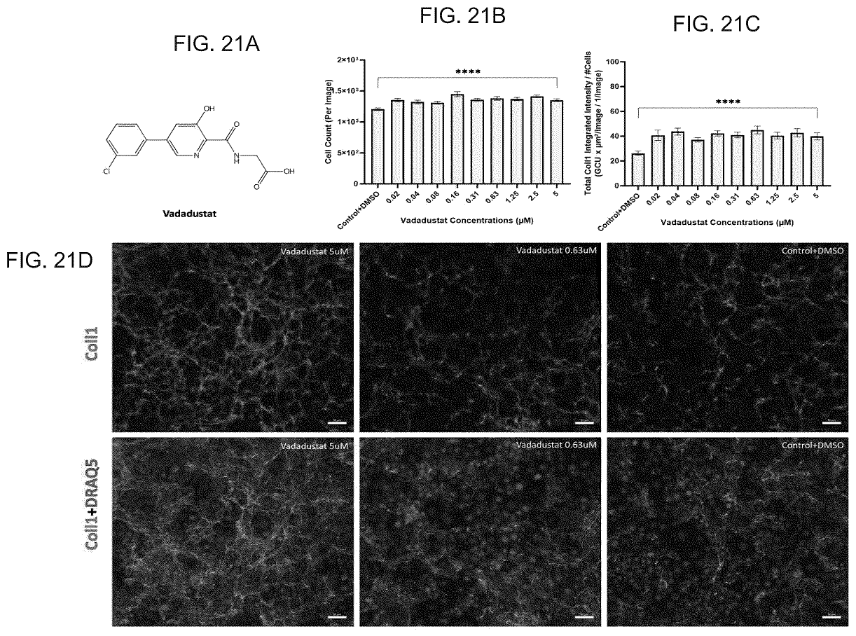

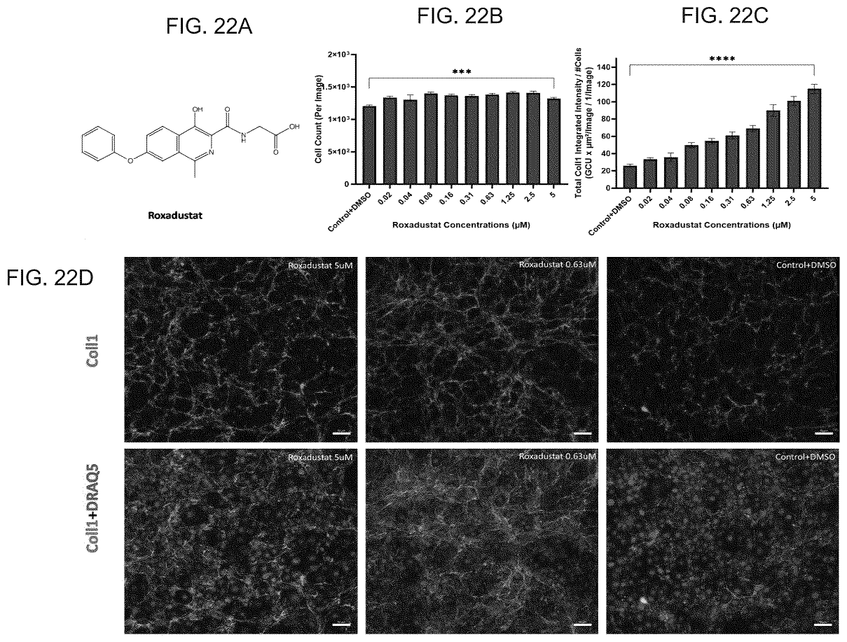

Examples 4 and 5 of the Examples section which follows show that increased levels of collagen can be obtained by increasing the activity of HIF-1α using an agent which inhibits the hydroxylation reaction of HIF-1α by the HIF-PHD enzyme. Thus, the iron chelator deferoxamine mesylate (DFO), increased Collagen-I production in MG-63 cells, even in the presence of low concentrations of DFO such as 0.15 μM, with maximal levels in the presence of 10-20 μM DFO (FIGS. 14A-B). Similarly, the present inventors have tested the ability of various small molecules which inhibit the hydroxylation of HIF-1α by HIF-PHD, such as Daprodustat (FIG. 18A), Enarodustat (FIG. 19A), Molidustat (FIG. 20A), Vadadustat (FIG. 21A) and Roxadustat (FIG. 22A) to increase production of collagen. These small molecules significantly increased Collagen-I levels in NIH/3T3 cells without or with only minimal-associated cell toxicity. Thus, using the HIF activators exemplified in the Examples section, over accumulation of Collagen I was evident in cells of Bovine and Rat origin. In addition, overexpression of Collagen I and III was evident in human cells (e.g., MG63 cells).

According to an aspect of some embodiments of the invention, there is provided an ex vivo method of increasing collagen production in cells. The method is effected by contacting the cells with an agent that increases an amount of Hypoxia-inducible factor-1 (HIF-1) in the cells, the amount being selected to cause an increase in the amount of collagen production in the cells.

The term “HIF-1”, as used herein, includes both the heterodimer complex and the subunits thereof, HIF-1α and HIF-1. The HIF-1 heterodimer consists of two helix-loop-helix proteins; these are termed HIF-1α, which is the oxygen-responsive component (see, e.g., Genbank accession no. Q16665 or NP_001521.1 (SEQ ID NO: 5)), and HIF-1β (see, e.g., Genbank accession no. NP_001659.1 (SEQ ID NO: 11)). The latter is also known as the aryl hydrocarbon receptor nuclear translocator (ARNT). Preferably, the term refers to the human form of HIF-1α. (see, e.g., Genbank Accession No. NM_001530 (SEQ ID NO: 12)).

According to some embodiments of the invention, the collagen which is produced in the cells is collagen type I.

According to some embodiments of the invention, the collagen which is produced in the cells is collagen type III.

Collagen Type I is a fibril-forming collagen found in most connective tissues and is abundant in bone, cornea, dermis and tendon. It forms a triple helix composed of two alpha1 (α1) chains and one alpha2 (α2) chain.

The human gene encoding pro-alpha1 chain of type I collagen is designated as COL1A1, and is also known as OI1, OI2, OI3, OI4, EDSC, CAFYD, or EDSARTH1.

The human gene encoding pro-alpha2 chain of type I collagen is designated as COL1A2, and is also known as OI4, EDSCV, or EDSARTH2.

Following are non-limiting sequences of type I collagen from human, which can be upregulated in a cell according to some embodiments of the invention.

Collagen alpha-1(I) chain isoform X1: mRNA sequence (GenBank Accession No. XM_011524341.2 (SEQ ID NO: 17), polypeptide sequence (GenBank Accession No. XP_011522643.1 (SEQ ID NO: 18).

Collagen alpha-1(I) chain isoform X2: mRNA sequence (GenBank Accession No. XM_005257058.5 (SEQ ID NO: 19), polypeptide sequence (GenBank Accession No. XP_005257115.2 (SEQ ID NO: 20).

Collagen alpha-1(I) chain isoform X3: mRNA sequence (GenBank Accession No. XM_005257059.5 (SEQ ID NO: 21), polypeptide sequence (GenBank Accession No. XP_005257116.2 (SEQ ID NO: 22).

Collagen alpha-2(I) chain precursor: mRNA sequence (GenBank Accession No. NM_000089.4 (SEQ ID NO: 23), polypeptide sequence (GenBank Accession No. NP_000080.2 (SEQ ID NO: 24).

Collagen Type III is a fibrillar collagen, an extracellular matrix protein that is found in extensible connective tissues such as skin, lung, uterus, intestine and the vascular system, frequently in association with type I collagen. Collagen type III has a long triple-helical domain formed of three alpha 1 chains.

The human gene encoding the pro-alpha1 chains of type III collagen is designated COL3A1, and is also known as EDS4A, EDSVASC, or PMGEDSV.

Following are non-limiting sequences of type III collagen from human, which can be upregulated in a cell according to some embodiments of the invention.

Collagen alpha-1(III) chain preproprotein mRNA sequence (GenBank Accession No. NM_000090.4 (SEQ ID NO: 25), polypeptide sequence (GenBank Accession No. NP_000081.2 (SEQ ID NO: 26).

As used herein the term “organoid” refers to an artificial three-dimensional aggregate of live cells of at least two cell types.

The organoids are typically between 100-2000 μm in diameter (for example between 200-1000 μm in diameter and may comprise between about 500-100,000 cells (for example between 1,000 and 75,000 cells).

In one embodiment, the organoid comprises only human cells.

In another embodiment, the organoid comprises non-human cells.

As used herein the phrase “embryoid bodies” (EBs) refers to three dimensional multicellular aggregates of differentiated and undifferentiated cells derivatives of three embryonic germ layers.

As used herein the phrase “an agent that increases an amount of Hypoxia-inducible factor-1 (HIF-1) in the cells” refers to any molecule which increases the expression of HIF-1 in a cell, increases the stability of HIF-1 in a cell, and/or prevents degradation of HIF-1, to thereby increase activity of HIF-1 in the cell.

The molecule which increases activity of HIF-1 includes, but is not limited to, a small molecule, a polynucleotide, or a peptide.

According to some embodiments of the invention, preventing degradation of HIF-1 is by preventing tagging of HIF-1 for degradation by the proteasome.

According to some embodiments of the invention, contacting comprises culturing the cells in a medium comprising an activator of HIF-1.

According to some embodiments of the invention, the method is a non-invasive method.

According to an aspect to some embodiments of the invention, there is provided a method of inducing tissue regeneration or cell repair in a subject in need thereof comprising administering to the subject an HIF-1 activator in an amount (e.g., a therapeutically effective amount) which brings about an up-regulation of collagen, thereby inducing tissue regeneration or cell repair.

As used herein the phrase “therapeutically effective amount” refers to an amount of an active ingredient (e.g., a HIF-1 activator) effective to upregulate collagen levels to thereby induce tissue regeneration or cell repair in the subject in need thereof.

Determination of a therapeutically effective amount is well within the capability of those skilled in the art, especially in light of the detailed disclosure provided herein.

According to some embodiments of the invention, the method of inducing tissue regeneration or cell repair is effected in-vivo.

According to some embodiments of the invention, the subject is a human subject.

According to some embodiments of the invention, the HIF activator is added in order to produce collagen for tissue repair.

According to some embodiments of the invention, the HIF activator is added in order to produce collagen for bone tissue repair.

According to some embodiments of the invention, the HIF activator is added in order to produce collagen for oral tissue repair.

According to some embodiments of the invention, the HIF activator is used in in order to produce collagen for dental medicine.

According to some embodiments of the invention, the HIF activator is added in order to produce collagen for skin tissue repair.

According to some embodiments of the invention, the HIF activator is added in order to produce collagen for wound healing.

According to some embodiments of the invention, the HIF activator is added in order to produce collagen for wound healing of diabetic and/or chronic wounds.

The HIF activator of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the HIF activator accountable for the biological effect.

According to some embodiments of the invention the HIF activator is comprised in a medical dressing.

Medical dressings suitable for use in the methods of some embodiments of the invention for contacting a wound with the HIF activator can be any material that is biologically acceptable and suitable for placing over any wound such as a burn, or a surface lesion of the skin or the oral mucosa or teeth of the mouth. In exemplary embodiments, the medical dressing may be a woven or non-woven fabric of synthetic or non-synthetic fibers, or any combination thereof. The medical dressing may also comprise a support, such as a polymer foam, a natural or man-made sponge, a gel or a membrane that may absorb or have disposed thereon the HIF activator or a therapeutic composition comprising same. A gel suitable for use as a support for the HIF activator composition of some embodiments of the invention is KY™ [sodium carboxymethylcellulose 7H 4F (Hercules, Inc., Wilmington, Del.)].

A film, a natural or synthetic polymer, or a rigid or malleable material that is known to one of ordinary skill in the art as being acceptable for insertion in the mouth of a human or animal can place the HIF activator according to some embodiments of the invention in contact with a tooth or a lesion of the oral mucosa.

In some embodiments of the invention the support of the medical dressing is a gauze. The gauze may be absorbent and can be wetted with the HIF activator before applying the gauze to an infected wound or other site.

According to some embodiments of the invention, the HIF activator is soaked or impregnated in the medical dressing.

For example, when using a medical dressing with a gauze, the gauze may be impregnated with the therapeutic composition and then dried. This allows the impregnated dressing to be stored for later use, or to avoid excessively dampening an injured area.

According to some embodiments of the invention, the HIF activator is absorbed on the surface of the medical dressing.

For example, HIF activator or a therapeutic composition comprising same can be absorbed on the surface of the support material of the medical dressing. The HIF activator or a therapeutic composition comprising same may be applied to the surface by wetting the surface with a solution of the HIF activator or the therapeutic composition comprising same and drying the support to deposit the HIF activator and/or the therapeutic composition comprising same thereon.

It is noted that a concentration of the HIF activator or the composition comprising same that is effective for promoting wound healing and/or repair may be attained when the dressing is wetted by the patient's body.

According to some embodiments of the invention, the subject is a non-human subject.

According to an aspect to some embodiments of the invention, there is provided a cell culture comprising non-human cells and a culture medium which comprises an activator of Hypoxia-inducible factor-1 (HIF-1).

According to some embodiments of the invention, the cells comprise non-human cells.

According to some embodiments of the invention, the non-human cells comprise mammalian livestock cells.

As used herein the phrase “mammalian livestock” refers to a domesticated mammalian animal which is typically used as a source of food, such as meat and/or milk.

According to some embodiments of the invention, the mammalian livestock is a ruminant mammalian livestock.

According to some embodiments of the invention, the mammalian livestock is a non-ruminant mammalian livestock.

According to some embodiments of the invention, the ruminant mammalian livestock is selected from the group consisting of a Bovinae subfamily, sheep (ovine), goat, deer, and camel.

According to some embodiments of the invention, the ruminant mammalian livestock of the Bovinae subfamily is cattle or a yak.

According to some embodiments of the invention, the ruminant mammalian livestock of the Bovinae subfamily is cattle.

According to some embodiments of the invention, the cattle is buffalo, bison or cow (bovine).

According to some embodiments of the invention, the mammalian livestock is cow (bovine).

According to some embodiments of the invention, the cattle is cow (bovine).

According to some embodiments of the invention, the non-ruminant mammalian livestock is selected from the group pig, rabbit, and horse.

As used herein the term “HIF activator” or “activator of HIF-1”, which are interchangeably used herein refers to any agent capable of increasing the activity of HIF-1 in inducing production i.e., expression of collagen in a cell.

According to some embodiments of the invention, the activator of HIF-1 is provided in an amount which brings about an up-regulation of collagen as compared to the level of collagen in the absence of the activator of HIF-1.

Assays for detecting and monitoring the level of collagen in a cell include, but are not limited to immunological detection assays with collagen-specific antibodies such as immunofluorescence, immunohistochemistry, or Western blot. Collagen specific antibodies are available from a number of manufacturers, such as anti-Collagen-I antibody Abcam catalogue number ab138492, anti-Collagen I antibody Abcam catalogue number ab34710, recombinant rabbit anti-mouse Collagen-I antibody [EPR24331-53] (Abcam catalogue number ab270993), Collagen I Antibody (MA1-26771, Thermo Fisher Scientific).

Additionally or alternatively collagen fibers can be detected using collagen-specific stains such as Picrosirius Red Stain, which stains Collagen-I and Collagen-III.

Briefly, to visualize the extent of ECM formation, cells are first fixed, e.g., using paraformaldehyde (PFA), e.g., at a concentration of 3-5%, washed and air-dried. Then, the cells are stained with Sirius Red dye (e.g., 400 μl of Sirius Red dye at a concentration of 0.1% in saturated picric acid) for about 1 hour with mild shaking. The stained cell layers are then washed with acid (e.g., about 0.01 N HCl) to remove all non-bounded dye. Photographs are taken under confocal microscope using polarized light.

It should be noted that since induction of collagen production in a cell requires a sufficient amount of HIF-1α subunit, the HIF-1 activator according to some embodiments of the invention is an agent which increases the amount of HIF-1α and/or the stability of HIF-1α in a cell.

According to some embodiments of the invention the amount of HIF-1α in a cell can be increased by preventing degradation of HIF-1 via the proteasome.

Since tagging of HIF-1α to degradation via the proteasome involves hydroxylation of a Proline residue on HIF-1α by a HIF-prolyl hydroxylase (PHD) enzyme, the HIF-1 activator can prevent binding between HIP-1 and a HIF-PHD.

According to some embodiments of the invention, the HIF-1 activator prevents binding between HIF-prolyl hydroxylase (PHD) and the HIF-1.

According to some embodiments of the invention, the HIF-1 activator is an inhibitor of a HIF-PHD enzyme.

As used herein the phrase “HIF-prolyl hydroxylase (PHD)” refers an enzyme which catalyzes the post-translational formation of hydroxyproline in hypoxia-inducible factor (HIF) alpha proteins.

According to some embodiments of the invention, the HIF-PHD is HIF-PHD2 (also known as EGLN1; “egl-9 family hypoxia inducible factor 1”, HPH2, PHD2, SM20, ECYT3, HALAH, HPH-2, HIFPH2, ZMYND6, or C1orf12). For example, the human PHD2 (EGLN1) is provided by GenBank Accession No. Q9GZT9.1 (SEQ ID NO: 9).

According to some embodiments of the invention, the HIF-PHD is PHD1 (EGLN2) (also known as “egl-9 family hypoxia inducible factor 2”, EIT6, EIT-6, HPH-1, HPH-3, HIFPH1, HIF-PH1). For example, the human PHD1 (EGLN2) is provided by GenBank Accession No. Q96KS0 (SEQ ID NO: 8).

According to some embodiments of the invention, the HIF-PHD is PHD3 (EGLN3) (also known as “egl-9 family hypoxia inducible factor 3”, HIFPH3; HIFP4H3). For example, the human PHD3 is provided by GenBank Accession No. Q9H6Z9 (SEQ ID NO: 10).

According to a specific embodiment, the HIF-PHD is not HIF-PHD4 (P4HTM prolyl 4-hydroxylase, transmembrane, also known as PH4; PH-4; PHD4; EGLN4; HIDEA; HIFPH4; P4H-TM).

Both Liu et al (ACS Med. Chem. Lett. 2018, 9, 1193-1198) and Hirota, K. (Biomedicines, 2021, 9, 468. hypertext transfer protocols://doi(dot)org/10(dot)3390/biomedicines9050468) discloses agents that inhibit the interaction between HIF and PHD thereby increasing the stability of HIF.

According to some embodiments of the invention, the HIF-1 activator comprises a peptide agent.

According to some embodiments of the invention, the peptide comprises at least 15 and no more than 25 consecutive amino acid residues of the HIF-1α polypeptide set forth by SEQ ID NO: 5, e.g., at least 16 and no more than 25 consecutive amino acid residues of the HIF-1α polypeptide set forth by SEQ ID NO: 5, e.g., at least 17 and no more than 25 consecutive amino acid residues of the HIF-1α polypeptide set forth by SEQ ID NO: 5, e.g., at least 18 and no more than 25 consecutive amino acid residues of the HIF-1α polypeptide set forth by SEQ ID NO: 5, e.g., at least 19 and no more than 25 consecutive amino acid residues of the HIF-1α polypeptide set forth by SEQ ID NO: 5, e.g., at least 20 and no more than 25 consecutive amino acid residues of the HIF-1α polypeptide set forth by SEQ ID NO: 5, e.g., at least 21 and no more than 24 consecutive amino acid residues of the HIF-1α polypeptide set forth by SEQ ID NO: 5, e.g., at least 19 and no more than 21 consecutive amino acid residues of the HIF-1α polypeptide set forth by SEQ ID NO: 5.

According to some embodiments of the invention, the peptide comprises the hydroxylation site of 4-hydroxyproline which corresponds to the proline amino acid residue at position 564 of SEQ ID NO: 5 (HIF-1α).

According to some embodiments of the invention, the peptide comprises the C-terminal VHL (von Hippel-Lindau) recognition site of HIF-1α as set forth by SEQ ID NO: 13 (corresponds to amino acids at position 556-572 of SEQ ID NO: 5).

According to some embodiments of the invention, the peptide comprises the hydroxylation site of 4-hydroxyproline (which corresponds to Proline 564 of SEQ ID NO: 5) within the at least 15 and no more than 25 consecutive amino acid residues of the HIF-1α polypeptide set forth by SEQ ID NO: 5, e.g., within the at least 16 and no more than 25 consecutive amino acid residues of the HIF-1α polypeptide set forth by SEQ ID NO: 5, e.g., within the at least 17 and no more than 25 consecutive amino acid residues of the HIF-1α polypeptide set forth by SEQ ID NO: 5, e.g., within the at least 18 and no more than 25 consecutive amino acid residues of the HIF-1α polypeptide set forth by SEQ ID NO: 5, e.g., within the at least 19 and no more than 25 consecutive amino acid residues of the HIF-1α polypeptide set forth by SEQ ID NO: 5, e.g., within the at least 20 and no more than 25 consecutive amino acid residues of the HIF-1α polypeptide set forth by SEQ ID NO: 5, e.g., within the at least 21 and no more than 24 consecutive amino acid residues of the HIF-1α polypeptide set forth by SEQ ID NO: 5, e.g., within the at least 19 and no more than 21 consecutive amino acid residues of the HIF-1α polypeptide set forth by SEQ ID NO: 5.

According to some embodiments of the invention, the peptide comprises the C-terminal VHL (von Hippel-Lindau) recognition site of HIF-1α as set forth by SEQ ID NO: 13 within the at least 17 and no more than 25 consecutive amino acid residues of the HIF-1α polypeptide set forth by SEQ ID NO: 5, e.g., within at least 18 and no more than 25 consecutive amino acid residues of the HIF-1α polypeptide set forth by SEQ ID NO: 5, e.g., within at least 19 and no more than 25 consecutive amino acid residues of the HIF-1α polypeptide set forth by SEQ ID NO: 5, e.g., within at least 20 and no more than 25 consecutive amino acid residues of the HIF-1α polypeptide set forth by SEQ ID NO: 5, e.g., within at least 21 and no more than 25 consecutive amino acid residues of the HIF-1α polypeptide set forth by SEQ ID NO: 5, e.g., within at least 22 and no more than 25 consecutive amino acid residues of the HIF-1α polypeptide set forth by SEQ ID NO: 5, e.g., within at least 19 and no more than 23 consecutive amino acid residues of the HIF-1α polypeptide set forth by SEQ ID NO: 5, e.g., within at least 20 and no more than 22 consecutive amino acid residues of the HIF-1α polypeptide set forth by SEQ ID NO: 5, e.g., at least 19 and no more than 21 consecutive amino acid residues of the HIF-1α polypeptide set forth by SEQ ID NO: 5.

According to some embodiments of the invention, the peptide is set forth by SEQ ID NO: 4.

According to some embodiments of the invention, the peptide comprises at least 15 and no more than 25 consecutive amino acid residues of the EPAS1 (HIF-2α) polypeptide set forth by SEQ ID NO: 6.

According to some embodiments of the invention, the peptide comprises at least 15 and no more than 24 consecutive amino acid residues of the EPAS1 (HIF-2α) polypeptide set forth by SEQ ID NO: 6, e.g., at least 16 and no more than 25 consecutive amino acid residues of the EPAS1 polypeptide set forth by SEQ ID NO: 6, e.g., at least 17 and no more than 25 consecutive amino acid residues of the EPAS1 polypeptide set forth by SEQ ID NO: 6, e.g., at least 18 and no more than 25 consecutive amino acid residues of the polypeptide set forth by SEQ ID NO: 6, e.g., at least 19 and no more than 25 consecutive amino acid residues of the EPAS1 polypeptide set forth by SEQ ID NO: 6, e.g., at least 20 and no more than 25 consecutive amino acid residues of the EPAS1 polypeptide set forth by SEQ ID NO: 6, e.g., at least 21 and no more than 24 consecutive amino acid residues of the EPAS1 polypeptide set forth by SEQ ID NO: 6, e.g., at least 19 and no more than 21 consecutive amino acid residues of the EPAS1 polypeptide set forth by SEQ ID NO: 6.

According to some embodiments of the invention, the peptide comprises the hydroxylation site of 4-hydroxyproline which corresponds to the proline amino acid residue at position 531 of SEQ ID NO: 6 (EPAS1).

According to some embodiments of the invention, the peptide comprises at least a portion of the N-terminal transactivation domain (NTAD) as set forth by SEQ ID NO: 14 (which corresponds to amino acids at positions 496-542 of SEQ ID NO: 6).

According to some embodiments of the invention, the at least a portion of the NTAD comprises proline at amino acid position 531 of SEQ ID NO: 6.

According to some embodiments of the invention, the peptide comprises the hydroxylation site of 4-hydroxyproline (which corresponds to Proline 531 of SEQ ID NO: 6 (EPAS1)) within the at least 15 and no more than 25 consecutive amino acid residues of the EPAS1 polypeptide set forth by SEQ ID NO: 6, e.g., within the at least 16 and no more than 25 consecutive amino acid residues of the EPAS1 polypeptide set forth by SEQ ID NO: 6, e.g., within the at least 17 and no more than 25 consecutive amino acid residues of the EPAS1 polypeptide set forth by SEQ ID NO: 6, e.g., within the at least 18 and no more than 25 consecutive amino acid residues of the EPAS1 polypeptide set forth by SEQ ID NO: 6, e.g., within the at least 19 and no more than 25 consecutive amino acid residues of the EPAS1 polypeptide set forth by SEQ ID NO: 6, e.g., within the at least 20 and no more than 25 consecutive amino acid residues of the EPAS1 polypeptide set forth by SEQ ID NO: 6, e.g., within the at least 21 and no more than 24 consecutive amino acid residues of the EPAS1 polypeptide set forth by SEQ ID NO: 6, e.g., within the at least 19 and no more than 21 consecutive amino acid residues of the EPAS1 polypeptide set forth by SEQ ID NO: 6.

According to some embodiments of the invention, the peptide comprises at least a portion of the NTAD of EPAS1 which comprises proline at amino acid position 531 of SEQ ID NO: 6 within the at least 17 and no more than 25 consecutive amino acid residues of the EPAS1 polypeptide set forth by SEQ ID NO: 6, e.g., within at least 18 and no more than 25 consecutive amino acid residues of the EPAS1 polypeptide set forth by SEQ ID NO: 6, e.g., within at least 19 and no more than 25 consecutive amino acid residues of the EPAS1 polypeptide set forth by SEQ ID NO: 6, e.g., within at least 20 and no more than 25 consecutive amino acid residues of the EPAS1 polypeptide set forth by SEQ ID NO: 6, e.g., within at least 21 and no more than 25 consecutive amino acid residues of the EPAS1 polypeptide set forth by SEQ ID NO: 6, e.g., within at least 22 and no more than 25 consecutive amino acid residues of the EPAS1 polypeptide set forth by SEQ ID NO: 6, e.g., within at least 19 and no more than 23 consecutive amino acid residues of the EPAS1 polypeptide set forth by SEQ ID NO: 6, e.g., within at least 20 and no more than 22 consecutive amino acid residues of the EPAS1 polypeptide set forth by SEQ ID NO: 6, e.g., at least 19 and no more than 21 consecutive amino acid residues of the EPAS1 polypeptide set forth by SEQ ID NO: 6.

According to some embodiments of the invention, the peptide is set forth by SEQ ID NO: 2.

Methods of qualifying suitable peptides for their ability to prevent binding between HIF-prolyl hydroxylase (PHD) and the HIF-1 are known in the art, and include, for example, a competitive binding assay, a fluorescence polarization (FP) assay, Iso Thermal Calorimetry (ITC), Nuclear Magnetic Resonance (NMR) spectroscopy, or Surface plasmon resonance (SPR).

For example, for a fluorescence polarization (FP) assay the peptide-of-interest can be labeled (e.g., by a fluorescent label such as FITC) and following incubation with an isolated protein (e.g., HIF-PHD in this case) the level of bound peptide is determined. A non-limiting example of a fluorescence polarization (FP) assay is shown in FIG. 13.

Peptides capable of inhibiting prolyl hydroxylase domain (PHD) are disclosed in Kwon et al., Bioorganic & Medicinal Chemistry Letters 21 (2011) 4325-4328. Such peptides may serve to activate HIF. Methods of identifying Prolyl Hydroxylase 2 inhibitors are disclosed by Lei et al., 2015, ACS Med. Chem. Lett. 6, 1236-1240.

According to some embodiments of the invention, the peptide further comprises a cell penetrating peptide.

As used herein “cell penetrating peptide” abbreviated as “CPP” refers to a peptide that is capable of crossing a biological membrane. Cell penetrating peptides are also called cell-permeable peptides, protein-transduction domains (PTD) or membrane-translocation sequences (MTS). CPPs have the ability to translocate in vitro, ex-vivo and/or in vivo the cell membranes of a cell of interest (e.g., mammalian cell or a cell of an edible animal) and enter into cells and/or cell nuclei, and directs a conjugated moiety of interest to a desired cellular destination.

Several proteins and their peptide derivatives have been found to comprise cell internalization properties including but not limited to the Human Immunodeficiency Virus type 1 (HIV-I) protein Tat (Ruben et al J. Virol. 63, 1-8 (1989)), the herpes virus tegument protein VP22 (Elliott and O'Hare, Cell 88, 223-233 (1997)), Penetratin (Derossi et al, J. Biol. Chem. 271, 18188-18193 (1996)), protegrin 1 (PG-I) anti-microbial peptide SynB (Kokryakov et al, FEBS Lett. 327, 231-236 (1993)) and the basic fibroblast growth factor (Jans, Faseb J. 8, 841-847 (1994)). These carrier peptides are typically highly cationic and arginine or lysine rich. Indeed, synthetic poly-arginine peptides have been shown to be internalized with a high level of efficiency (Futaki et al, J. MoI. Recognit. 16, 260-264 (2003); Suzuki et al, J. Biol. Chem. (2001)).

According to some embodiments, the CPP is selected from the group consisting of Human Immunodeficency Virus type 1 (HIV-I) protein Tat, the herpes virus tegument protein VP22, Penetratin, protegrin 1 (PG-I) anti-microbial peptide SynB, the basic fibroblast growth factor, synthetic poly-arginine peptide, or peptide derivative thereof possessing cell internalization properties.

According to some embodiments, the CPP is derived from the defensin (chain A) protein [GenBank Accession No. 2LR3_A (SEQ ID NO: 7)].

According to some embodiments, the CPP comprising an amino acid sequence which does not exceed 30 amino acids in length, comprising an RGFRRR loop as set forth by SEQ ID NO: 30 and having at least 80% global sequence identity to SEQ ID NO: 1.

According to some embodiments, the amino acid sequence of the CPP comprising an RGFRRR loop as set forth by SEQ ID NO: 30, and does not exceed 29 amino acids in length, e.g., does not exceed 28 amino acids in length, e.g., does not exceed 27 amino acids in length, e.g., does not exceed 26 amino acids in length, e.g., does not exceed 25 amino acids in length, e.g., does not exceed 24 amino acids in length, e.g., does not exceed 23 amino acids in length, e.g., does not exceed 22 amino acids in length, e.g., does not exceed 21 amino acids in length, e.g., does not exceed 20 amino acids in length, e.g., does not exceed 19 amino acids in length, e.g., does not exceed 18 amino acids in length, e.g., does not exceed 17 amino acids in length, e.g., does not exceed 16 amino acids in length, wherein the amino acid sequence of the CPP having at least 80% global sequence identity to SEQ ID NO: 1.

According to some embodiments, the amino acid sequence of the CPP comprising at least 8 amino acids which comprise the RGFRRR loop set forth by SEQ ID NO: 30, e.g., at least 9 amino acids, e.g., at least 10 amino acids, e.g., at least 11 amino acids, e.g., least 12 amino acids, e.g., at least 13 amino acids, e.g., at least 14 amino acids, e.g., at least 15 amino acids, e.g., at least 16 amino acids, wherein the amino acid sequence of the CPP having at least 80% global sequence identity to SEQ ID NO: 1.

According to some embodiments, the amino acid sequence of the CPP comprising at least 8 amino acids which comprise the RGFRRR loop set forth by SEQ ID NO: 30, and no more than 30 amino acids in length, e.g., at least 9 amino acids, e.g., at least 8 amino acids, e.g., at least 10 amino acids, e.g., at least 11 amino acids, e.g., at least 12 amino acids, e.g., at least 13 amino acids, e.g., at least 14 amino acids, e.g., at least 15 amino acids, e.g., at least 16 amino acids and no more than 30 amino acids in length, e.g., no more than 29, 28, 27, 26, 25, 24, 23, 22, 21, 20, 19, 18, 17 amino acids in length, wherein the amino acid sequence of the CPP having at least 80% global sequence identity to SEQ ID NO: 1.

According to some embodiments, the amino acid sequence of the CPP has at least 81% global sequence identity to SEQ ID NO:1, e.g., at least 82%, 83%, 84%, 85%, 86%, 87%, 88%, 89%, 90%, 91%, 92%, 93%, 94%, 95%, 96%, 97%, 98%, 99% or more global sequence identity to SEQ ID NO:1.

As used herein the term “global” with respect to sequence identity refers to identity over an entire amino acid sequence of the peptide of some embodiments of the invention (e.g., the CPP of the peptide agent) and not over a portion thereof.

Also included in the scope of the invention are natural and synthetic homologs (e.g., having at least 80%, 81%, 82%, 83%, 84%, 85%, 86%, 87%, 88%, 89%, 90%, 91%, 92%, 93%, 94%, 95%, 96%, 97%, 98%, 99% global sequence identity to any of the peptides referred to or described herein).

The degree of identity between two or more sequences can be determined using various known sequence comparison tools. Following is a non-limiting description of such tools which can be used along with some embodiments of the invention.

Pairwise global alignment was defined by S. B. Needleman and C. D. Wunsch, “A general method applicable to the search of similarities in the amino acid sequence of two proteins” Journal of Molecular Biology, 1970, pages 443-53, volume 48).

For example, when starting from a polypeptide sequence and comparing to other polypeptide sequences, the EMBOSS-6.0.1 Needleman-Wunsch algorithm (available from emboss(dot)sourceforge(dot)net/apps/cvs/emboss/apps/needle(dot)html) can be used to find the optimum alignment (including gaps) of two sequences along their entire length—a “Global alignment”. Default parameters for Needleman-Wunsch algorithm (EMBOSS-6.0.1) include: gapopen=10; gapextend=0.5; datafile=EBLOSUM62; brief=YES.

According to some embodiments of the invention, the parameters used with the EMBOSS-6.0.1 tool (for protein-protein comparison) include: gapopen=8; gapextend=2; datafile=EBLOSUM62; brief=YES.

According to some embodiments of the invention, the threshold used to determine homology using the EMBOSS-6.0.1 Needleman-Wunsch algorithm is 80%, 81%, 82%, 83%, 84%, 85%, 86%, 87%, 88%, 89%, 90%, 91%, 92%, 93%, 94%, 95%, 96%, 97%, 98%, 99%, or 100%.

When starting from a polypeptide sequence and comparing to polynucleotide sequences, the OneModel FramePlus algorithm [Halperin, E., Faigler, S. and Gill-More, R. (1999)—FramePlus: aligning DNA to protein sequences. Bioinformatics, 15, 867-873) (available from biocceleration(dot)com/Products(dot)html] can be used with following default parameters: model=frame+_p2n.model mode=local.

According to some embodiments of the invention, the parameters used with the OneModel FramePlus algorithm are model=frame+_p2n.model, mode=qglobal.

According to some embodiments of the invention, the threshold used to determine homology using the OneModel FramePlus algorithm is 80%, 81%, 82%, 83%, 84%, 85%, 86%, 87%, 88%, 89%, 90%, 91%, 92%, 93%, 94%, 95%, 96%, 97%, 98%, 99%, or 100%.

According to some embodiments, determination of the degree of identity employs the Smith-Waterman algorithm (for protein-protein comparison or nucleotide-nucleotide comparison).

Default parameters for GenCore 6.0 Smith-Waterman algorithm include: model=sw.model.

According to some embodiments of the invention, the threshold used to determine homology using the Smith-Waterman algorithm is 80%, 81%, 82%, 83%, 84%, 85%, 86%, 87%, 88%, 89%, 90%, 91%, 92%, 93%, 94%, 95%, 96%, 97%, 98%, 99%, or 100%.

According to some embodiments, the CPP is set forth by SEQ ID NO:1.

According to some embodiments, the CPP is non-toxic to mammalian cells.

According to some embodiments, the CPP is non-toxic to cells of an edible animal.

According to some embodiments, the CPP is not used on or is not contacted with fungal cells.

According to some embodiments of the invention, the CPP is attached to the N-terminal of the peptide.

According to some embodiments of the invention, the CPP is attached to the C-terminal of the peptide.

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated amide bonds (—N(CH3)-CO—), ester bonds (—C(═O)—O—), ketomethylene bonds (—CO—CH2-), sulfinylmethylene bonds (—S(═O)—CH2-), α-aza bonds (—NH—N(R)—CO—), wherein R is any alkyl (e.g., methyl), amine bonds (—CH2-NH—), sulfide bonds (—CH2-S—), ethylene bonds (—CH2-CH2-), hydroxyethylene bonds (—CH(OH)—CH2-), thioamide bonds (—CS—NH—), olefinic double bonds (—CH═CH—), fluorinated olefinic double bonds (—CF═CH—), retro amide bonds (—NH—CO—), peptide derivatives (—N(R)—CH2-CO—), wherein R is the “normal” side chain, naturally present on the carbon atom.

These modifications can occur at any of the bonds along the peptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The peptides of some embodiments of the invention may also include one or more modified amino acids or one or more non-amino acid monomers (e.g. fatty acids, complex carbohydrates etc.).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids. Tables 1 and 2 below list naturally occurring amino acids (Table 1), and non-conventional or modified amino acids (e.g., synthetic, Table 2) which can be used with some embodiments of the invention.

TABLE 1

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino | Xaa | X |
| acid as above | | |

Table 1.

TABLE 2

| Non-conventional | | Non-conventional | |
|---|---|---|---|
| amino acid | Code | amino acid | Code |
| ornithine | Orn | hydroxyproline | Hyp |
| α-aminobutyric acid | Abu | aminonorbornyl- | Norb |
| | | carboxylate | |
| D-alanine | Dala | aminocyclopropane- | Cpro |
| | | carboxylate | |
| D-arginine | Darg | N-(3-guanidinopropyl)glycine | Narg |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl) glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylornithine | Dnmorn | L-N-methylornithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl- | Nmhphe |
| | | homophenylalanine | |
| α-naphthylalanine | Anap | N-methyl-α-naphthylalanine | Nmanap |
| penicillamine | Pen | N-methylpenicillamine | Nmpen |
| γ-aminobutyric acid | Gabu | N-methyl-γ-aminobutyrate | Nmgabu |
| cyclohexylalanine | Chexa | N-methyl-cyclohexylalanine | Nmchexa |
| cyclopentylalanine | Cpen | N-methyl-cyclopentylalanine | Nmcpen |
| α-amino-α-methylbutyrate | Aabu | N-methyl-α-amino-α- | Nmaabu |
| | | methylbutyrate | |
| α-aminoisobutyric acid | Aib | N-methyl-α-aminoisobutyrate | Nmaib |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | Dmile | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylornithine | Dmorn | L-α-methylornithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl- | Mhphe |
| | | homophenylalanine | |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobutyrate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2- | Nbhm | N-(N-(2,2-diphenylethyl) | Nnbhm |
| diphenylethyl)glycine | | carbamylmethyl-glycine | |
| N-(3,3- | Nbhe | N-(N-(3,3-diphenylpropyl) | Nnbhe |
| diphenylpropyl)glycine | | carbamylmethyl-glycine | |
| 1-carboxy-1-(2,2-diphenyl | Nmbc | 1,2,3,4- | Tic |
| ethylamino)cyclopropane | | tetrahydroisoquinoline-3- | |
| | | carboxylic acid | |
| phosphoserine | pSer | phosphothreonine | pThr |
| phosphotyrosine | pTyr | O-methyl-tyrosine | |
| 2-aminoadipic acid | | hydroxylysine | |

Table 2.

The peptides of some embodiments of the invention are preferably utilized in a linear form, although it will be appreciated that in cases where cyclicization does not severely interfere with peptide characteristics, cyclic forms of the peptide can also be utilized.

Since the present peptides are preferably utilized in therapeutics or diagnostics which require the peptides to be in soluble form, the peptides of some embodiments of the invention preferably include one or more non-natural or natural polar amino acids, including but not limited to serine and threonine which are capable of increasing peptide solubility due to their hydroxyl-containing side chain.

The peptides of some embodiments of the invention are preferably utilized in a linear form, although it will be appreciated that in cases where cyclicization does not severely interfere with peptide characteristics, cyclic forms of the peptide can also be utilized.

The peptides of some embodiments of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A preferred method of preparing the peptide compounds of some embodiments of the invention involves solid phase peptide synthesis.

Large scale peptide synthesis is described by Andersson Biopolymers 2000; 55(3):227-50.

Additionally or alternatively, the HIF-1 activator of some embodiments of the invention is an agent which reduces or prevents hydroxylation of HIF-1α by a HIF-PHD enzyme.

According to some embodiments of the invention, the HIF activator reduces hydroxylation of HIF-1 under normoxia by the HIF-PHD enzyme.

The term “normoxia” as used herein refers to a level of oxygen (measured in percentage of oxygen in the form of O₂) in a tissue or a cell culture which, in the absence of a HIF-activator, results in degradation of HIF-1α by the proteasome.

The tissue can be part of the organism or can be cultured and/or generated ex-vivo, i.e., outside of the organism.

According to some embodiments of the invention, the tissue which is generated ex-vivo comprises an organoid or an embryoid body.

It should be noted that when HIF1α and HIF1β expression was measured in cultured HeLa cells in the presence of 0% to 20% oxygen, a maximal response of HIF1 expression was observed in the presence of 0.5% oxygen, with a half maximal expression at 1.5-2% oxygen and a significantly low expression in the presence of 4% oxygen (Jiang B H, et al., 1996. Am. J. Physiol. 271: C1172-80. “Hypoxia-inducible factor 1 levels vary exponentially over a physiologically relevant range of O₂ tension”). Thus, while normoxia conditions may include a tissue oxygen level of 20-21%, normal tissues can be ordinarily maintained at an oxygen level which is higher than 4%, e.g., at 3-7% oxygen.

According to some embodiments of the invention, the normoxia conditions under which the HIF activator reduces hydroxylation of HIF-1α by the HIF-PHD enzyme comprise oxygen (O₂) levels in the range of 3-21%, 4-21%, 5-21%, 6-21%, 7-21%, 8-21%, 9-21%, 10-21%, 11-21%, 12-21%, 13-21%, 14-21%, 15-21%, 16-21%, 17-21%, 18-21%, 19-21%, or 20-21%.

According to some embodiments of the invention, the HIF activator, which reduces the level of hydroxylation of HIF-1α by the HIF-PHD enzyme under normoxia, increases the amount of the HIF-1α subunit in the cytoplasm to form a heterodimer with the HIF-1β subunit and thereby activate collagen production by the cell.

According to some embodiments of the invention, the HIF activator, which prevents hydroxylation of HIF-1α by a HIF-PHD enzyme under normoxia, prevents degradation of HIF-1α and thereby enhances collagen production by the cell,

According to some embodiments the agent which increases expression of HIF-1 is a small molecule.

According to some embodiments the HIF activator is a small molecule agent.

According to some embodiments of the invention, the HIF activator is an iron chelator.

Non-limiting examples of iron chelators are known in the art, such as Desferrioxamine (DFX), dipyridyl (DP), and Quercetin (QUE),

According to some embodiments of the invention, the iron chelator is Deferoxamine mesylate (DFO).

According to some embodiments of the invention, the iron chelator is provided at a concentration range between 1 nM-100 μM. According to some embodiments of the invention, the iron chelator is provided at a concentration range between 0.15-20 μM, e.g., at a concentration range between 10-20 μM.

The 2-oxoglutarate (2-OG) molecule is an essential co-substrate of the HIF-PHD enzyme in the hydroxylation reaction of HIF-1α.

According to some embodiments of the invention, the HIF activator comprises an analogue of 2-oxoglutarate (2-OG),

The term “analogue” as used herein refers to a molecule which is structurally similar and/or otherwise exhibit the same functionality as the small molecule of some embodiments of the invention, characterized, in a most preferred embodiment, by their possession of at least one of the abovementioned biological activities of activating HIF-1, e.g., by inhibiting HIF-PHD activity as described herein.

According to some embodiments of the invention, 2-OG analogue is a competitive inhibitor of 2-OG capable of preventing hydroxylation of HIF-1α by HIF-PHD.

According to some embodiments of the invention, the 2-OG analogue is capable of reducing or preventing the hydroxylation of HIF-1α by the HIF-PHD enzyme under normoxia,

According to some embodiments of the invention, the small molecule is selected from the group consisting of Roxadustat (FG-4592), Daprodustat, Molidustat, Enarodustat (JTZ-951) and Vadadustat, or derivatives thereof.

According to some embodiments of the invention, Roxadustat is provided at a concentration range of 1 nM-50 μM, e.g., at a concentration of 0.02-5 μM.

According to some embodiments of the invention, Daprodustat is provided at a concentration range of 1 nM-50 μM, e.g., at a concentration of 0.02-5 μM.

According to some embodiments of the invention, Molidustat is provided at a concentration range of 1 nM-50 μM, e.g., at a concentration of 0.02-5 μM.

According to some embodiments of the invention, Enarodustat is provided at a concentration range of 1 nM-50 μM, e.g., at a concentration of 0.02-5 μM.

According to some embodiments of the invention, Vadadustat is provided at a concentration range of 1 nM-50 μM, e.g., at a concentration of 0.02-5 μM.

According to some embodiments of the invention, the HIF activator is not DMOG, IOX, BIQ, DFX, or cobalt chloride heptahydrate.

According to some embodiments of the invention, the HIF-1 activator is provided at a concentration which does not induce cell death.

Cell viability or cell death can be evaluated using the live-dead viability assay (e.g., catalogue number 04511-1KT-F Merck, Germany) For example, cells are incubated in a medium with or without the HIF-1 activator, and following a predetermined time period (e.g., about 48 hours), fluorescein diacetate (e.g., at a concentration of about 6.6 μg/mL) and propidium iodide (e.g., at a concentration of about 5 μg/mL) are prepared in a culture medium (e.g., DMEM) and added to the cells. Confocal images are captured after addition of the dyes, e.g., with ZEISS LSM 900, Laser Scanning Microscope. Fluorescence readouts are obtained with excitation/emission (ex/em) of 490/515 nm and 535/617 nm for green and red, respectively.

Additionally or alternatively, cellular ATP levels can be quantified using the cell-titer assay (Promega, WI, USA cat #G7570).

Additionally or alternatively, cell viability can detected using Alamar blue assay which includes the non-toxic, cell-permeable compound resazurin, which upon entering living cells, is reduced to resorufin, a compound that is red in color and highly fluorescent. Alamar blue stain can be obtained e.g., from Enco (Petach Tikvah, Israel). For example, cultured cells can be stained by replacing the cell medium with Alamar blue (e.g., diluted 1:10 in the cell medium), and incubating for about 4 hours (37° C., 5% CO₂). Then, fluorescence readouts can be obtained using the Spark multimode microplate reader (Tecan) at excitation/emission 540±20 nm/590±20 nm. Cell viability can be calculated as the percentage difference between treated cells and untreated (control) cells at 590±20 nm emission.

According to some embodiments of the invention, the small molecule agent is ML228 or a derivative thereof.

According to some embodiments of the invention, ML228 is provided at a concentration range of 20 nM-20 μM, e.g., 0.05-20 μM, e.g., at a concentration of 0.019-20 μM, e.g., at a concentration of 0.3-5 μM. Activators of HIF may be small molecule agents are disclosed in Nagle and Zhou, Curr Pharm Des. 2006; 12(21): 2673-2688, the contents of which are incorporated herein by reference.

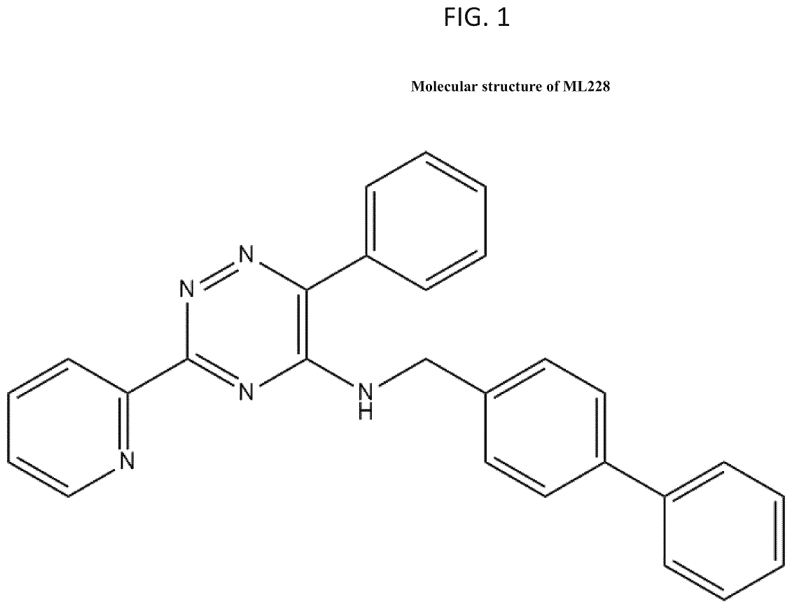

According to a particular embodiment, the HIF activator is ML228 (FIG. 1). Details of this activator are provided in Theriault et al Bioorg Med Chem Lett. 2012 Jan. 1; 22(1): 76-81. doi:10.1016/j.bmcl.2011.11.077, the contents of which are incorporated herein by reference.

Additional HIF-prolyl hydroxylase inhibitors are disclosed in US Patent Application No. 20210353612, the contents of which are incorporated herein by reference.

Agents which increase activity HIF are also disclosed in US Patent Application No. 20220143211 and US Patent Application No. 20210162008, the contents of which are incorporated herein by reference.

Additional contemplated HIF activators include Desferrioxamine (DFO) and 1-mimosine (1-mim) (World Wide Web(dot)pnas(dot)org/doi/full/10(dot)1073/pnas(dot)0708474105). According to some embodiments of the invention, the HIF-1 activator comprises an agent which downregulates expression of HIF-PHD.

Downregulation of HIF-PHD can be effected on the genomic and/or the transcript level using a variety of molecules which interfere with transcription and/or translation [e.g., RNA silencing agents (e.g., antisense, siRNA, shRNA, micro-RNA), Ribozyme, DNAzyme and a CRISPR system (e.g. CRISPR/Cas)], or on the protein level using e.g., antagonists, enzymes that cleave the polypeptide and the like.

According to specific embodiments the downregulating agent is an RNA silencing agent.

As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs.

In one embodiment, the RNA silencing agent is capable of inducing RNA interference.

In another embodiment, the RNA silencing agent is capable of mediating translational repression.

According to an embodiment of the invention, the RNA silencing agent is specific to the target RNA (e.g., HIF-PHD) and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence-specific post-transcriptional gene silencing in animals mediated by short interfering RNAs (siRNAs).

Following is a detailed description on RNA silencing agents that can be used according to specific embodiments of the present invention.

DsRNA, siRNA and shRNA—The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to downregulate protein expression from mRNA.

According to one embodiment dsRNA longer than 30 bp (base pairs) are used. Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

According to some embodiments of the invention, dsRNA is provided in cells where the interferon pathway is not activated, see for example Billy et al., PNAS 2001, Vol 98, pages 14428-14433; and Diallo et al, Oligonucleotides, Oct. 1, 2003, 13(5): 381-392. doi:10.1089/154545703322617069.

According to an embodiment of the invention, the long dsRNA are specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 base pairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is suggested to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned, the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-CAAGAGA-3′ and 5′-UUACAA-3′ (International Patent Application Nos. WO2013126963 and WO2014107763). It will be recognized by one of skill in the art that the resulting single chain oligonucleotide forms a stem-loop or hairpin structure comprising a double-stranded region capable of interacting with the RNAi machinery.

Synthesis of RNA silencing agents suitable for use with some embodiments of the invention can be effected as follows. First, the HIF-PHD mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (World Wide Web(dot)ambion(dot)com/techlib/tn/91/912(dot)html).

Second, potential target sites are compared to an appropriate genomic database (e.g., human, mouse, rat etc.) using any sequence alignment software, such as the BLAST software available from the NCBI server (World Wide Web(dot)ncbi(dot)nlm(dot)nih(dot)gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

It will be appreciated that, and as mentioned hereinabove, the RNA silencing agent of some embodiments of the invention need not be limited to those molecules containing only RNA, but further encompasses chemically-modified nucleotides and non-nucleotides.

miRNA and miRNA mimics—According to another embodiment the RNA silencing agent may be a miRNA.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses(dot)fwdarw(dot)humans) and have been shown to play a role in development, homeostasis, and disease etiology.

Below is a brief description of the mechanism of miRNA activity.

Genes coding for miRNAs are transcribed leading to production of an miRNA precursor known as the pri-miRNA. The pri-miRNA is typically part of a polycistronic RNA comprising multiple pri-miRNAs. The pri-miRNA may form a hairpin with a stem and loop. The stem may comprise mismatched bases.

The hairpin structure of the pri-miRNA is recognized by Drosha, which is an RNase III endonuclease. Drosha typically recognizes terminal loops in the pri-miRNA and cleaves approximately two helical turns into the stem to produce a 60-70 nucleotide precursor known as the pre-miRNA. Drosha cleaves the pri-miRNA with a staggered cut typical of RNase III endonucleases yielding a pre-miRNA stem loop with a 5′ phosphate and ˜2 nucleotide 3′ overhang. It is estimated that approximately one helical turn of stem (˜10 nucleotides) extending beyond the Drosha cleavage site is essential for efficient processing. The pre-miRNA is then actively transported from the nucleus to the cytoplasm by Ran-GTP and the export receptor Ex-portin-5.

The double-stranded stem of the pre-miRNA is then recognized by Dicer, which is also an RNase III endonuclease. Dicer may also recognize the 5′ phosphate and 3′ overhang at the base of the stem loop. Dicer then cleaves off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. miRNA* sequences may be found in libraries of cloned miRNAs but typically at lower frequency than the miRNAs.

Although initially present as a double-stranded species with miRNA*, the miRNA eventually becomes incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-7 of the miRNA.

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al. (2005, Nat Genet 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

The term “microRNA mimic” or “miRNA mimic” refers to synthetic non-coding RNAs that are capable of entering the RNAi pathway and regulating gene expression. miRNA mimics imitate the function of endogenous miRNAs and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-O,4′-C-ethylene-bridged nucleic acids (ENA)). For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA.

Preparation of miRNAs mimics can be effected by any method known in the art such as chemical synthesis or recombinant methods.

It will be appreciated from the description provided herein above that contacting cells with a miRNA may be effected by transfecting the cells with e.g. the mature double stranded miRNA, the pre-miRNA or the pri-miRNA.

The pre-miRNA sequence may comprise from 45-90, 60-80 or 60-70 nucleotides.

The pri-miRNA sequence may comprise from 45-30,000, 50-25,000, 100-20,000, 1,000-1,500 or 80-100 nucleotides.

Antisense—Antisense is a single stranded RNA designed to prevent or inhibit expression of a gene by specifically hybridizing to its mRNA. Downregulation of a HIF-PHD can be effected using an antisense polynucleotide capable of specifically hybridizing with an mRNA transcript encoding HIF-PHD.

Design of antisense molecules which can be used to efficiently downregulate a HIF-PHD must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Jääskeläinen et al. Cell Mol Biol Lett. (2002) 7(2):236-7; Gait, Cell Mol Life Sci. (2003) 60(5):844-53; Martino et al. J Biomed Biotechnol. (2009) 2009:410260; Grijalvo et al. Expert Opin Ther Pat. (2014) 24(7):801-19; Falzarano et al, Nucleic Acid Ther. (2014) 24(1):87-100; Shilakari et al. Biomed Res Int. (2014) 2014: 526391; Prakash et al. Nucleic Acids Res. (2014) 42(13):8796-807 and Asseline et al. J Gene Med. (2014) 16(7-8):157-65]

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)]. Such algorithms have been successfully used to implement an antisense approach in cells.

In addition, several approaches for designing and predicting efficiency of specific oligonucleotides using an in vitro system were also published (Matveeva et al., Nature Biotechnology 16: 1374-1375 (1998)].

Thus, the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Another agent capable of downregulating a HIF-PHD expression is a ribozyme molecule capable of specifically cleaving an mRNA transcript encoding a HIF-PHD. Ribozymes are known for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)].

Another agent capable of downregulating a HIF-PHD is a DNAzyme molecule capable of specifically cleaving an mRNA transcript or DNA sequence of the HIF-PHD. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine: pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al., 20002, Abstract 409, Ann Meeting Am Soc Gen Ther; World Wide Web(dot)asgt(dot)org). In another application, DNAzymes complementary to bcr-ab1 oncogenes were successful in inhibiting the oncogenes expression in leukemia cells, and lessening relapse rates in autologous bone marrow transplant in cases of CML and ALL.

Nucleic acid agents can also operate at the DNA level as summarized infra.

Downregulation of HIF-PHD can also be achieved by inactivating the gene encoding HIF-PHD via introducing targeted mutations involving loss-of function alterations (e.g., point mutations, deletions and insertions) in the gene structure.

As used herein, the phrase “loss-of-function alterations” refers to any mutation in the DNA sequence of a gene (e.g., encoding a HIF-PHD) which results in downregulation of the expression level and/or activity of the expressed product, i.e., the mRNA transcript and/or the translated protein.

According to specific embodiments loss-of-function alteration of a gene may comprise at least one allele of the gene. According to specific embodiments loss-of-function alteration of a gene comprises both alleles of the gene.

The term “allele” as used herein, refers to any of one or more alternative forms of a gene locus, all of which alleles relate to a trait or characteristic. In a diploid cell or organism, the two alleles of a given gene occupy corresponding loci on a pair of homologous chromosomes.

Methods of introducing nucleic acid alterations to a gene of interest are well known in the art [see for example Menke D. Genesis (2013) 51:-618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases, PB transposases and genome editing by engineered nucleases. Agents for introducing nucleic acid alterations to a gene of interest can be designed publically available sources or obtained commercially from Transposagen, Addgene and Sangamo Biosciences.

According to some embodiments of the invention, the HIF-1 activator comprises an agent which downregulates expression of HIF-PHD by means of genome editing.

Following is a description of various exemplary methods used to introduce nucleic acid alterations to a gene of interest (e.g., a gene encoding HIF-PHD) and agents for implementing same that can be used according to specific embodiments of the present invention.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDR) and non-homologous end-joining (NFfEJ). NFfEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, CA).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through hypertext transfer protocol://World Wide Web(dot)talendesign(dot)org). TALEN can also be designed and obtained commercially from e.g., Sangamo Biosciences™ (Richmond, CA).

CRISPR-Cas system—Many bacteria and archea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRISPR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

In order to use the CRISPR system, both gRNA and Cas9 should be expressed in a target cell. The insertion vector can contain both cassettes on a single plasmid or the cassettes are expressed from two separate plasmids. CRISPR plasmids are commercially available such as the px330 plasmid from Addgene.

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, electroporated into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After electroporation and positive selection, homologously targeted clones are identified. Next, a second targeting vector that contains a region of homology with the desired mutation is electroporated into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

Transposases—As used herein, the term “transposase” refers to an enzyme that binds to the ends of a transposon and catalyzes the movement of the transposon to another part of the genome.

As used herein the term “transposon” refers to a mobile genetic element comprising a nucleotide sequence which can move around to different positions within the genome of a single cell. In the process the transposon can cause mutations and/or change the amount of a DNA in the genome of the cell.

A number of transposon systems that are able to also transpose in cells e.g. vertebrates have been isolated or designed, such as Sleeping Beauty [Izsvák and Ivics Molecular Therapy (2004) 9, 147-156], piggyBac [Wilson et al. Molecular Therapy (2007) 15, 139-145], Tol2 [Kawakami et al. PNAS (2000) 97 (21): 11403-11408] or Frog Prince [Miskey et al. Nucleic Acids Res. December 1, (2003) 31(23): 6873-6881]. Generally, DNA transposons translocate from one DNA site to another in a simple, cut-and-paste manner. Each of these elements has their own advantages, for example, Sleeping Beauty is particularly useful in region-specific mutagenesis, whereas Tol2 has the highest tendency to integrate into expressed genes. Hyperactive systems are available for Sleeping Beauty and piggyBac. Most importantly, these transposons have distinct target site preferences, and can therefore introduce sequence alterations in overlapping, but distinct sets of genes. Therefore, to achieve the best possible coverage of genes, the use of more than one element is particularly preferred. The basic mechanism is shared between the different transposases, therefore we will describe piggyBac (PB) as an example.

PB is a 2.5 kb insect transposon originally isolated from the cabbage looper moth, Trichoplusia ni. The PB transposon consists of asymmetric terminal repeat sequences that flank a transposase, PBase. PBase recognizes the terminal repeats and induces transposition via a “cut-and-paste” based mechanism, and preferentially transposes into the host genome at the tetranucleotide sequence TTAA. Upon insertion, the TTAA target site is duplicated such that the PB transposon is flanked by this tetranucleotide sequence. When mobilized, PB typically excises itself precisely to reestablish a single TTAA site, thereby restoring the host sequence to its pretransposon state. After excision, PB can transpose into a new location or be permanently lost from the genome.

Typically, the transposase system offers an alternative means for the removal of selection cassettes after homologous recombination quit similar to the use Cre/Lox or Flp/FRT. Thus, for example, the PB transposase system involves introduction of a targeting vector with 3′ and 5′ homology arms containing the mutation of interest, two PB terminal repeat sequences at the site of an endogenous TTAA sequence and a selection cassette placed between PB terminal repeat sequences. Positive selection is applied and homologous recombinants that contain targeted mutation are identified. Transient expression of PBase removes in conjunction with negative selection results in the excision of the selection cassette and selects for cells where the cassette has been lost. The final targeted allele contains the introduced mutation with no exogenous sequences.

For PB to be useful for the introduction of sequence alterations, there must be a native TTAA site in relatively close proximity to the location where a particular mutation is to be inserted.

Genome editing using recombinant adeno-associated virus (rAAV) platform—this genome-editing platform is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross and/or subtle endogenous gene alterations in a cell. rAAV genome editing has the advantage in that it targets a single allele and does not result in any off-target genomic alterations. rAAV genome editing technology is commercially available, for example, the rAAV GENESIS™ system from Horizon™ (Cambridge, UK).

It will be appreciated that the agent can be a mutagen that causes random mutations and the cells exhibiting downregulation of the expression level and/or activity of HIF-PHD may be selected.

The mutagens may be, but are not limited to, genetic, chemical or radiation agents. For example, the mutagen may be ionizing radiation, such as, but not limited to, ultraviolet light, gamma rays or alpha particles. Other mutagens may include, but not be limited to, base analogs, which can cause copying errors; deaminating agents, such as nitrous acid; intercalating agents, such as ethidium bromide; alkylating agents, such as bromouracil; transposons; natural and synthetic alkaloids; bromine and derivatives thereof; sodium azide; psoralen (for example, combined with ultraviolet radiation). The mutagen may be a chemical mutagen such as, but not limited to, ICR191, 1,2,7,8-diepoxy-octane (DEO), 5-azaC, N-methyl-N-nitrosoguanidine (MNNG) or ethyl methane sulfonate (EMS).

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and western blot analysis and immunohistochemistry.

In addition, one ordinarily skilled in the art can readily design a knock-in/knock-out construct including positive and/or negative selection markers for efficiently selecting transformed cells that underwent a homologous recombination event with the construct. Positive selection provides a means to enrich the population of clones that have taken up foreign DNA. Non-limiting examples of such positive markers include glutamine synthetase, dihydrofolate reductase (DHFR), markers that confer antibiotic resistance, such as neomycin, hygromycin, puromycin, and blasticidin S resistance cassettes. Negative selection markers are necessary to select against random integrations and/or elimination of a marker sequence (e.g. positive marker). Non-limiting examples of such negative markers include the herpes simplex-thymidine kinase (HSV-TK) which converts ganciclovir (GCV) into a cytotoxic nucleoside analog, hypoxanthine phosphoribosyltransferase (HPRT) and adenine phosphoribosyltransferase (APRT).

According to some embodiments of the invention, the HIF-1 activator comprises an agent which downregulates expression of HIF-PHD at the polypeptide level.

According to specific embodiments the agent capable of downregulating the HIF-PHD polypeptide is an antibody or antibody fragment capable of specifically binding HIF-PHD. Preferably, the antibody specifically binds at least one epitope of a HIF-PHD.

The term “antibody” as used in this invention includes intact molecules as well as functional fragments thereof (that are capable of binding to an epitope of an antigen).

As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

According to a specific embodiment, the antibody fragments include, but are not limited to, single chain, Fab, Fab′ and F(ab′)₂ fragments, Fd, Fcab, Fv, dsFv, scFvs, diabodies, minibodies, nanobodies, Fab expression library or single domain molecules such as VH and VL that are capable of binding to an epitope of the antigen in an HLA restricted manner.

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv (scFv), a disulfide-stabilized Fv (dsFv), an Fab, an Fab′, and an F(ab′)2, or antibody fragments comprising the Fc region of an antibody.

As used herein, the terms “complementarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR LI or LI; CDR L2 or L2; and CDR L3 or L3).

As used herein, the “variable regions” and “CDRs” may refer to variable regions and CDRs defined by any approach known in the art, including combinations of approaches.

As the HIF-PHD polypeptide is localized intracellularly, an antibody or antibody fragment capable of specifically binding HIF-PHD is typically an intracellular antibody (also known as “intrabody” or “intrabodies”).

Intrabodies are essentially SCA (single chain antibody) to which intracellular localization signals have been added (e.g., ER, mitochondrial, nuclear, cytoplasmic). To prepare an intracellular antibody expression vector, the cDNA encoding the antibody light and heavy chains specific for the target protein of interest are isolated, typically from a hybridoma that secretes a monoclonal antibody specific for the marker. Once a monoclonal antibody specific for the marker protein is identified (e.g., either a hybridoma-derived monoclonal antibody or a recombinant antibody from a combinatorial library), DNAs encoding the light and heavy chains of the monoclonal antibody are isolated by standard molecular biology techniques. For hybridoma derived antibodies, light and heavy chain cDNAs can be obtained, for example, by PCR amplification or cDNA library screening. For recombinant antibodies, such as from a phage display library, cDNA encoding the light and heavy chains can be recovered from the display package (e.g., phage) isolated during the library screening process and the nucleotide sequences of antibody light and heavy chain genes are determined. For example, many such sequences are disclosed in Kabat, E. A., et al. (1991) Sequences of Proteins of Immunological Interest, Fifth Edition, U.S. Department of Health and Human Services, NIH Publication No. 91-3242 and in the “Vbase” human germline sequence database. Once obtained, the antibody light and heavy chain sequences are cloned into a recombinant expression vector using standard methods.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Another agent which can be used along with some embodiments of the invention to downregulate HIF-PHD is an aptamer. As used herein, the term “aptamer” refers to double stranded or single stranded RNA molecule that binds to specific molecular target, such as a protein. Various methods are known in the art which can be used to design protein specific aptamers. The skilled artisan can employ SELEX (Systematic Evolution of Ligands by Exponential Enrichment) for efficient selection as described in Stoltenburg R, Reinemann C, and Strehlitz B (Biomolecular engineering (2007) 24(4):381-403).

Another agent capable of downregulating HIF-PHD would be any molecule which binds to and/or cleaves HIF-PHD. Such molecules can be a small molecule, HIF-PHD antagonists, or HIF-PHD inhibitory peptide.

Another contemplated agent which can be used to downregulate HIF-PHD includes a proteolysis-targeting chimaera (PROTAC). Such agents are heterobifunctional, comprising a ligand which binds to a ubiquitin ligase (such as E3 ubiquitin ligase) and a ligand to HIF-PHD and optionally a linker connecting the two ligands. Binding of the PROTAC to the target protein leads to the ubiquitination of an exposed lysine on the target protein, followed by ubiquitin proteasome system (UPS)-mediated protein degradation.

It will be appreciated that a non-functional analogue of at least a catalytic or binding portion of HIF-PHD can be also used as an agent which downregulates HIF-PHD.

As descried above, the ex vivo method according to some embodiments of the invention is effected by contacting the cells with an agent that increases an amount of HIF-1 in the cells, the amount being selected to cause an increase in the amount of collagen production in the cells.

According to some embodiments of the invention, contacting comprises genetically modifying the cells so that they express exogenous HIF-1.

According to some embodiments of the invention, the cells are genetically modified to express collagen (e.g., exogenous collagen).

The term “exogenous” refers to a heterologous amino acid sequence which may not be naturally expressed within the cell (e.g., an amino acid sequence which is translated from a nucleic acid sequence derived from a different species) or which overexpression in the cell is desired.

Methods of genetically modifying cells to express an exogenous HIF-1 or exogenous collagen include, but are not limited to recombinant DNA technology using a an exogenous polynucleotide encoding the HIF-1 or the collagen amino acid sequence or genome editing techniques.

To express exogenous HIF-1 or collagen in cells, a polynucleotide sequence encoding a HIF-1α (SEQ ID NO: 5) and/or collagen (e.g., human collagen sequences set forth by SEQ ID NOs: 18, 20, 22, 24, 28 or 29) is preferably ligated into a nucleic acid construct suitable for expression in the cell-of-interest, such as a mammalian cell, a human cell, an animal cell, an edible animal cell, a mammalian livestock cell. Expression can be in any cell, examples include, but are not limited to, a fibroblast cell, or an osteoblast cell. Such a nucleic acid construct includes a promoter sequence for directing transcription of the polynucleotide sequence in the cell in a constitutive or inducible manner.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, a typical cloning vectors may also contain a transcription and translation initiation sequence, transcription and translation terminator and a polyadenylation signal. By way of example, such constructs will typically include a 5′ LTR, a (RNA binding site, a packaging signal, an origin of second-strand DNA synthesis, and a 3′ LTR or a portion thereof.

The nucleic acid construct of some embodiments of the invention typically includes a signal sequence for secretion of the peptide from a host cell in which it is placed. Preferably the signal sequence for this purpose is a mammalian signal sequence or the signal sequence of the polypeptide variants of some embodiments of the invention.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of HIF-1 and/or collagen mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

It will be appreciated that the individual elements comprised in the expression vector can be arranged in a variety of configurations. For example, enhancer elements, promoters and the like, and even the polynucleotide sequence(s) encoding a HIF-1 and/or collagen can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. 149: 51-60).

Recombinant viral vectors are useful for in vivo expression of HIF-1 and/or collagen since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of some embodiments of the invention into stem cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of some embodiments of the invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof, Other vectors can be used that are non-viral, such as cationic lipids, polylysine, and dendrimers.

Other than containing the necessary elements for the transcription and translation of the inserted coding sequence, the expression construct of some embodiments of the invention can also include sequences engineered to enhance stability, production, purification, yield or toxicity of the expressed peptide, For example, the expression of a fusion protein or a cleavable fusion protein comprising the HIF-1 and/or collagen protein of some embodiments of the invention and a heterologous protein can be engineered. Such a fusion protein can be designed so that the fusion protein can be readily isolated by affinity chromatography; e.g., by immobilization on a column specific for the heterologous protein. Where a cleavage site is engineered between the HIF-1 and/or collagen protein and the heterologous protein, the HIF-1 and/or collagen protein can be released from the chromatographic column by treatment with an appropriate enzyme or agent that disrupts the cleavage site [e.g., see Booth et al. (1988) Immunol. Lett, 19:65-70; and Gardella et al., (1990) J. Biol. Chem. 265:15854-15859].

As mentioned hereinabove, a variety of prokaryotic or eukaryotic cells can be used as host-expression systems to express the polypeptides of some embodiments of the invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the polypeptides of some embodiments of the invention.

Examples of bacterial constructs include the pET series of E. coli expression vectors [Studier et al. (1990) Methods in Enzymol. 185:60-89.(

In yeast, a number of vectors containing constitutive or inducible promoters can be used, as disclosed in U.S. Pat. No. 5,932,447. Alternatively, vectors can be used which promote integration of foreign DNA sequences into the yeast chromosome.

In cases where plant expression vectors are used, the expression of the coding sequence can be driven by a number of promoters. For example, viral promoters such as the 35S RNA and 19S RNA promoters of CaMV [Brisson et al. (1984) Nature 310:511-514], or the coat protein promoter to TMV [Takamatsu et al. (1987) EMBO J. 6:307-311] can be used. Alternatively, plant promoters such as the small subunit of RUBISCO [Coruzzi et al. (1984) EMBO J. 3:1671-1680 and Brogli et al., (1984) Science 224:838-843] or heat shock promoters, e.g., soybean hsp17.5-E or hsp17.3-B [Gurley et al. (1986) Mol. Cell. Biol. 6:559-565] can be used. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach, 1988, Methods for Plant Molecular Biology, Academic Press, NY, Section VIII, pp 421-463.

Other expression systems such as insects and mammalian host cell systems which are well known in the art and are further described hereinbelow can also be used by some embodiments of the invention.

Recovery of the recombinant polypeptide is effected following an appropriate time in culture. The phrase “recovering the recombinant polypeptide” refers to collecting the whole fermentation medium containing the polypeptide and need not imply additional steps of separation or purification. Notwithstanding the above, polypeptides of some embodiments of the invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

According to some embodiments of the invention, the cells are of an edible animal.

According to some embodiments of the invention, the non-human cells comprise non-human connective tissue cells.

As used herein, the term “connective tissue cells” refers to the various cell types that make up connective tissue. In some embodiments, connective tissue cells are selected from fibroblasts, cartilage cells, bone cells, fat cells and smooth muscle cells. In some embodiments, connective tissue cells are selected from the group consisting of chondrocytes, adipocytes, osteoblasts, osteocytes, myofibroblasts, satellite cells, myoblasts and myocytes. In some embodiments, connective tissue cells are selected from the group consisting of, adipocytes, osteoblasts, osteocytes, myofibroblasts, satellite cells, myoblasts and myocytes. In some embodiments, connective tissue cells are fibroblasts. In some embodiments, the fibroblasts are not embryonic fibroblasts. In some embodiments, the fibroblasts are embryonic fibroblasts. In some embodiments, the fibroblasts are fetal fibroblasts. In some embodiments, the fibroblasts are dermal fibroblasts. In some embodiments, connective tissue cells are fibroblasts or a cell type that can be differentiated from a fibroblast. In some embodiments, connective tissue cells are not mesenchymal stem cells (MSCs). In some embodiments, connective tissue cells are not cells derived from MSCs. In some embodiments, connective tissue cells are cell that cannot be derived from MSCs. In some embodiments, the cell type can be naturally differentiated form a fibroblast. In some embodiments, the cell type results from natural fibroblast differentiation. As used herein, the “term natural differentiation” is used to refer to a differentiation that occurs in nature and not a trans-differentiation such as can artificially be achieved in a laboratory. In some embodiments, the natural differentiation is not de-differentiation. In some embodiments, a cell type that can naturally be differentiated form a fibroblast is selected from the group consisting of: a chondrocyte, an adipocyte, an osteoblast, an osteocyte, a myofibroblast, a myoblast and a myocyte. In some embodiments, a cell type that can naturally be differentiated form a fibroblast is selected from the group consisting of: an adipocyte, an osteoblast, an osteocyte, a myofibroblast, a myoblast and a myocyte. In some embodiments, a cell type that can naturally be differentiated form a fibroblast is an adipocyte. In some embodiments, the connective tissue cell is not a pluripotent cell. In some embodiments, the connective tissue cell is not a mesenchymal stem cell.

In some embodiments, the connective tissue cells are mammalian cells. In some embodiments, the mammal is a bovine. In some embodiments, the bovine is a cow. In some embodiments, the connective tissue cells are avian cells. In some embodiments, the connective tissue cells are fish cells. In some embodiments, the connective tissue cells are from an edible animal. In some embodiments, the cells are from livestock animals. In some embodiments, a livestock animal is selected from a cow, a pig, a goat, a sheep, a chicken, a fish and a turkey. In some embodiments, a livestock animal is selected from a cow, a pig, a goat, a sheep, a chicken, a fish, a duck, a goose and a turkey. In some embodiments, a livestock animal is selected from a cow, a pig, a goat, a sheep, a chicken, a duck, a goose and a turkey. In some embodiments, the connective tissue cells are selected from avian cells and bovine cells. In some embodiments, the bovine cells are cow cells. In some embodiments, the avian cells are chicken cells. In some embodiments, the connective tissue cells are selected from cow cells and chicken cells. In some embodiments, the chicken cells are chicken fibroblasts. In some embodiments, the cow cells are cow fibroblasts. In some embodiments, the cells are immortalized. In some embodiments, the cells are not immortalized. In some embodiments, the cells are derived from primary cells.

According to some embodiments of the invention, the cells comprise non-human stem cells or progenitor cells.

According to some embodiments of the invention, the cells further comprise non-human muscle cells.

According to some embodiments of the invention, the cells are selected from the group consisting of fibroblasts, osteoblasts and adipocytes.

As described above, contacting can be by culturing the cells in a medium comprising the activator of the HIF-1.

According to some embodiments of the invention, the contacting is effected by culturing in a culture medium in the absence of serum.

According to some embodiments of the invention, the culturing is effected in the presence of an osteogenic differentiation medium.

According to some embodiments of the invention, the osteogenic differentiation medium comprises ascorbate-2-phosphate (e.g., at a concentration of about 50 μM) and β-glycerophosphate (e.g., at a concentration of about 10 mM).

The osteogenic differentiation medium can further include a basic culture medium such as minimum essential medium alpha (α-MEM) supplemented with serum or serum replacement (e.g., 10% heat-inactivated fetal bovine serum (FBS)), and L-glutamine (e.g., at a concentration of about 2 mM). The medium can optionally include antibiotic such as 100 U/mL penicillin and 100 μg/mL streptomycin.

For osteogenic differentiation osteoblast cells such as MG-63 cells cultured in α-MEM growth medium can be plated in culture wells (e.g., at a density of about 20,000 cells/well into a sterile 24-well plate). The cells are cultured allowed to adhere to the plate, e.g., for 1 day, and then are switched to an osteogenic differentiation medium. Seven to twenty-eight days after induction of differentiation, the production of extracellular calcified mineral matrix can be visualized by Alizarin Red staining (Sigma) and Picrosirius Red (Scytek).

According to some embodiments of the invention, the collagen comprises type I collagen.

According to some embodiments of the invention, the collagen comprises type III collagen.

According to some embodiments of the invention, the non-human cells comprise at least 10% more collagen compared to the non-human cells cultured in the culture medium in an absence of the activator of the HIF-1.

According to some embodiments of the invention, the non-human cells comprise at least 20%, at least 30%, at least 50%, at least 70%, at least 100% more collagen compared to the non-human cells cultured in the culture medium in an absence of the activator of the HIF-1.

For example, FIGS. 16A-B show an increase of 200-500% collagen as compared to the same cells when cultured in the absence of the activator of HIF-1.

According to some embodiments of the invention, the non-human cells comprise at least 2-folds more collagen compared to the non-human cells cultured in the culture medium in an absence of the activator of the HIF-1, e.g., at least at least 3-folds more, at least 4-folds more, at least 5-folds, more at least 6-folds more, at least 7-folds more, at least 9-folds more, at least 9-folds more, at least 10-folds more, at least 11-folds more, at least 12-folds more, at least 13-folds more, at least 14-folds more, at least 15-folds more, at least 16-folds more, at least 17-folds more, at least 18-folds more, at least 19-folds more, or at least 20-folds more collagen compared to the non-human cells cultured in the culture medium in an absence of the activator of the HIF-1.

According to some embodiments of the invention, the cells are non-genetically modified cells.

According to some embodiments of the invention, the cells are not genetically modified to express exogenous HIF-1.

According to some embodiments of the invention, the cells are not genetically modified to express exogenous Collagen.

According to an aspect of some embodiments of the invention, there is provided a method of generating collagen comprising:
(a) increasing collagen production according to the method of some embodiments of the invention; and
(b) isolating the collagen from the cells or from the medium.

According to some embodiments of the invention, the isolating the collagen is from the cells.

According to an aspect of some embodiments of the invention, there is provided a method of generating a food product comprising:
(a) increasing collagen production according to the method of some embodiments of the invention;
(b) isolating the cells from the medium;
(c) combining the cells or the collagen isolated from the cells with a food or feed product.

According to some embodiments of the invention, the food product comprises a cultured meat or cultured cells which can be combined with other substances to result in cultured meat.

As used herein the term “cultured meat” refers to in-vitro cultured animal cells processed to impart an organoleptic sensation and texture of meat.

The cultured meat product may include a variety of cells, including but not limited to adipocytes, muscle cells, blood cells, cartilage cells, bone cells, connective tissue cells, fibroblasts and/or cardiomyocytes.

Since collagen, as part of the ECM, can enhance the growth and/or differentiation of connective tissue cells, the addition of the collagen generated by the method of some embodiments of the invention or the HIF activators to a culture medium of cells for cultured meat can facilitate the process of generating cultured meat, and/or shorten the culturing period when compared to the culturing period in the absence of the added collagen.

According to some embodiments of the invention, the collagen isolated by the method of some embodiments of the invention is used for enhancing production of a cultured meat.

According to some embodiments of the invention, the collagen isolated by the method of some embodiments of the invention is included in a processed food product.

According to some embodiments of the invention, the collagen isolated by the method of some embodiments of the invention is used for generating gelatin.

According to some embodiments of the invention, the collagen isolated by the method of some embodiments of the invention is used as a wrap for a food product such as a cold cut or sausage.

According to some embodiments of the invention, the collagen isolated by the method of some embodiments of the invention is used as a wrap for a vegetarian cold cut or sausage which is made of a cultured meat.

According to some embodiments of the invention, the collagen isolated by the method of some embodiments of the invention is used in the food industry for improving texture of a food product by absorbing water and increasing the stickiness of the food product.

According to some embodiments of the invention, the in vitro cultured animal cells are mammalian livestock cells.

According to some embodiments of the invention, the in vitro cultured animal cells are bovine cells (though other cells can be included e.g., ovine, fish, porcine, avian etc.).

According to some embodiments of the invention, the method further comprising combining the cells or the collagen isolated from the cells with a taste modifying agent.

According to an aspect of some embodiments of the invention, there is provided a food product generated according to the method of some embodiments of the invention.

According to some embodiments of the invention, the collagen isolated by the method of some embodiments of the invention is used for replacing vegetable proteins to improve the texture properties of cultured-food.

According to some embodiments of the invention, the collagen isolated by the method of some embodiments of the invention is used as an emulsifier, especially in acidic food.

According to some embodiments of the invention, the collagen isolated by the method of some embodiments of the invention is as a carrier of a bioactive agent(s). Examples bioactive agents include, but are not limited to extracts of herbs, or antioxidant materials.

According to some embodiments of the invention, the collagen isolated by the method of some embodiments of the invention is used as a substitute for non-biodegradable plastic packaging materials in food packaging and preservation.

According to some embodiments of the invention, the HIF activator is formulated as a cosmetic in a cosmetic composition.

As used herein a “cosmetic composition” refers to a preparation which includes the active ingredients described hereinabove (e.g., the HIF activator) and additional chemical components such as physiologically suitable carriers and excipients. The purpose of a cosmetic composition is to facilitate administration of the active ingredient to an organism.

Hereinafter, the phrases “suitable carrier” used refer to a carrier or a diluent that does not cause significant irritation to an organism and does not abrogate the biological activity and properties of the HIF activator.

Herein the term “excipient” refers to an inert substance added to a cosmetic composition to further facilitate administration of an active ingredient. Examples, without limitation, of excipients include calcium carbonate, calcium phosphate, various sugars and types of starch, cellulose derivatives, gelatin, vegetable oils and polyethylene glycols.

The cosmetic composition may be applied in a local manner, for example, via administration of the cosmetic composition directly into a tissue region of a patient. Suitable routes of administration may, for example, include topical, subcutaneous and intradermal injections.

Cosmetic compositions of the present invention may be manufactured by processes well known in the art, e.g., by means of conventional mixing, dissolving, granulating, dragee-making, levigating, emulsifying, encapsulating, entrapping or lyophilizing processes.

Cosmetic compositions for use in accordance with the present invention thus may be formulated in conventional manner using one or more physiologically acceptable carriers comprising excipients and auxiliaries, which facilitate processing of the active ingredients into preparations. Proper formulation is dependent upon the administration approach chosen.

For injection, the active ingredients of the cosmetic composition may be formulated in aqueous solutions, preferably in physiologically compatible buffers such as Hank's solution, Ringer's solution, or physiological salt buffer.

Alternatively, the active ingredient may be in a powder form for constitution with a suitable vehicle, e.g., sterile, pyrogen-free water based solution, before use.

Determination of a therapeutically or cosmetically effective amount is well within the capability of those skilled in the art, especially in light of the detailed disclosure provided herein.

For any preparation used in the methods of the invention, the therapeutically or cosmetically effective amount or dose can be estimated initially from in vitro assays. Depending on the need dosing can be of a single or a plurality of administrations.

The amount of a composition to be administered will, of course, be dependent on the subject being treated, the severity of the affliction, the manner of administration, the judgment of the prescribing physician, etc.

The composition is preferably of high purity and substantially free of potentially harmful contaminants, e.g., at least National Food (NF) grade, generally at least analytical grade, and preferably at least pharmaceutical grade. To the extent that a given compound must be synthesized prior to use, such synthesis or subsequent purification shall preferably result in a product that is substantially free of any potentially contaminating toxic agents that may have been used during the synthesis or purification procedures.

### Epidermal Penetrants

In order to enhance the percutaneous absorption of the active ingredients (e.g., the HIF activator), one or more of a number of agents can be added to the cosmetic composition including, but not limited to, dimethylsulfoxide, dimethylacetamide, dimethylformamide, surfactants, azone, alcohol, acetone, propylene glycol and polyethylene glycol.

The cosmetic composition of some embodiments of the invention also includes a dermatologically acceptable carrier.

The phrase “dermatologically acceptable carrier”, refers to a carrier which is suitable for topical application onto the skin, i.e., keratinous tissue, has good aesthetic properties, is compatible with the active agents of the present invention and any other components, and is safe and non-toxic for use in mammals. An effective amount of carrier is selected from a range of about 50% to about 99.99%, preferably from about 80% to about 99.9%, more preferably from about 90% to about 98%, and most preferably from about 90% to about 95%, by weight, of the composition.

### Emulsions

The carrier utilized in the compositions of the invention can be in a wide variety of forms. These include emulsion carriers, including, but not limited to, oil-in-water, water-in-oil, water-in-oil-in-water, and oil-in-water-in-silicone emulsions, a cream, an ointment, an aqueous solution, a lotion or an aerosol. As will be understood by the skilled artisan, a given component will distribute primarily into either the water or oil/silicone phase, depending on the water solubility/dispersibility of the component in the composition.

Emulsions according to the present invention generally contain a pharmaceutically or cosmetically effective amount of an agent disclosed herein and a lipid or oil. Lipids and oils may be derived from animals, plants, or petroleum and may be natural or synthetic (i.e., man-made). Preferred emulsions also contain a humectant, such as glycerin. Emulsions will preferably further contain from about 1% to about 10%, more preferably from about 2% to about 5%, of an emulsifier, based on the weight of the carrier. Emulsifiers may be nonionic, anionic or cationic. Suitable emulsifiers are described in, for example, U.S. Pat. No. 3,755,560, issued to Dickert, et al. Aug. 28, 1973; U.S. Pat. No. 4,421,769, issued to Dixon, et al., Dec. 20, 1983; and McCutcheon's Detergents and Emulsifiers, North American Edition, pages 317-324 (1986).

The emulsion may also contain an anti-foaming agent to minimize foaming upon application to the keratinous tissue. Anti-foaming agents include high molecular weight silicones and other materials well known in the art for such use.

Suitable emulsions may have a wide range of viscosities, depending on the desired product form. Exemplary low viscosity emulsions, which are preferred, have a viscosity of about 50 centistokes or less, more preferably about 10 centistokes or less, most preferably about 5 centistokes or less. The emulsion may also contain an anti-foaming agent to minimize foaming upon application to the keratinous tissue. Anti-foaming agents include high molecular weight silicones and other materials well known in the art for such use.

One type of emulsion is a water-in-silicone emulsion. Water-in-silicone emulsions contain a continuous silicone phase and a dispersed aqueous phase. Preferred water-in-silicone emulsions of the present invention comprise from about 1% to about 60%, preferably from about 5% to about 40%, more preferably from about 10% to about 20%, by weight of a continuous silicone phase. The continuous silicone phase exists as an external phase that contains or surrounds the discontinuous aqueous phase described hereinafter.

The continuous silicone phase may contain a polyorganosiloxane oil. A preferred water-in-silicone emulsion system is formulated to provide an oxidatively stable vehicle for delivery of a pharmaceutically or cosmetically effective amount of an agent disclosed herein. The continuous silicone phase of these preferred emulsions comprises between about 50% and about 99.9% by weight of organopolysiloxane oil and less than about 50% by weight of a non-silicone oil. In an especially preferred embodiment, the continuous silicone phase comprises at least about 50%, preferably from about 60% to about 99.9%, more preferably from about 70% to about 99.9%, and even more preferably from about 80% to about 99.9%, polyorganosiloxane oil by weight of the continuous silicone phase, and up to about 50% non-silicone oils, preferably less about 40%, more preferably less than about 30%, even more preferably less than about 10%, and most preferably less than about 2%, by weight of the continuous silicone phase. These useful emulsion systems may provide more oxidative stability over extended periods of time than comparable water-in-oil emulsions containing lower concentrations of the polyorganosiloxane oil. Concentrations of non-silicone oils in the continuous silicone phase are minimized or avoided altogether so as to possibly further enhance oxidative stability of the active compound of the invention in the compositions. Water-in-silicone emulsions of this type are described in U.S. Pat. No. 5,691,380 to Mason et al., issued Nov. 25, 1997.

The organopolysiloxane oil for use in the composition may be volatile, non-volatile, or a mixture of volatile and non-volatile silicones. The term “nonvolatile” as used in this context refers to those silicones that are liquid under ambient conditions and have a flash point (under one atmospheric of pressure) of or greater than about 100 degrees Celsius. The term “volatile” as used in this context refers to all other silicone oils. Suitable organopolysiloxanes can be selected from a wide variety of silicones spanning a broad range of volatilities and viscosities. Examples of suitable organopolysiloxane oils include polyalkylsiloxanes, cyclic polyalkylsiloxanes, and polyalkylarylsiloxanes, which are known to those skilled in the art and commercially available.

The continuous silicone phase may contain one or more non-silicone oils. Concentrations of non-silicone oils in the continuous silicone phase are preferably minimized or avoided altogether so as to further enhance oxidative stability of the pharmaceutically effective agent in the compositions. Suitable non-silicone oils have a melting point of about 25° C. or less under about one atmosphere of pressure. Examples of non-silicone oils suitable for use in the continuous silicone phase are those well known in the chemical arts in topical personal care products in the form of water-in-oil emulsions, e.g., mineral oil, vegetable oils, synthetic oils, semisynthetic oils, etc.

Useful topical compositions of the present invention comprise from about 30% to about 90%, more preferably from about 50% to about 85%, and most preferably from about 70% to about 80% of a dispersed aqueous phase. The term “dispersed phase” is well-known to one skilled in the art it implies that the phase exists as small particles or droplets that are suspended in and surrounded by a continuous phase. The dispersed phase is also known as the internal or discontinuous phase. The dispersed aqueous phase is a dispersion of small aqueous particles or droplets suspended in and surrounded by the continuous silicone phase described hereinbefore. The aqueous phase can be water, or a combination of water and one or more water soluble or dispersible ingredients. Non-limiting examples of such optional ingredients include thickeners, acids, bases, salts, chelants, gums, water-soluble or dispersible alcohols and polyols, buffers, preservatives, sunscreening agents, colorings, and the like.

The topical compositions of the present invention typically comprise from about 25% to about 90%, preferably from about 40% to about 80%, more preferably from about 60% to about 80%, water in the dispersed aqueous phase by weight of the composition.

The water-in-silicone emulsions of the present invention preferably comprise an emulsifier. In a preferred embodiment, the composition contains from about 0.1% to about 10% emulsifier, more preferably from about 0.5% to about 7.5%, most preferably from about 1% to about 5%, emulsifier by weight of the composition. The emulsifier helps disperse and suspend the aqueous phase within the continuous silicone phase.

A wide variety of emulsifying agents can be employed herein to form the preferred water-in-silicone emulsion. Known or conventional emulsifying agents can be used in the composition, provided that the selected emulsifying agent is chemically and physically compatible with essential components of the composition, and provides the desired dispersion characteristics. Suitable emulsifiers include silicone emulsifiers, e.g., organically modified organopolysiloxanes, also known to those skilled in the art as silicone surfactants, non-silicon-containing emulsifiers, and mixtures thereof, known by those skilled in the art for use in topical personal care products.

Suitable emulsifiers are described, for example, in McCutcheon's, Detergents and Emulsifiers, North American Edition (1986), published by Allured Publishing Corporation; U.S. Pat. No. 5,011,681 to Ciotti et al., issued Apr. 30, 1991; U.S. Pat. No. 4,421,769 to Dixon et al., issued Dec. 20, 1983; and U.S. Pat. No. 3,755,560 to Dickert et al., issued Aug. 28, 1973.

Other preferred topical carriers include oil-in-water emulsions, having a continuous aqueous phase and a hydrophobic, water-insoluble phase (“oil phase”) dispersed therein. Examples of suitable carriers comprising oil-in-water emulsions are described in U.S. Pat. No. 5,073,371 to Turner, D. J. et al., issued Dec. 17, 1991, and U.S. Pat. No. 5,073,372, to Turner, D. J. et al., issued Dec. 17, 1991. An especially preferred oil-in-water emulsion, containing a structuring agent, hydrophilic surfactant and water, is described in detail hereinafter.

A preferred oil-in-water emulsion comprises a structuring agent to assist in the formation of a liquid crystalline gel network structure. The structuring agent may also function as an emulsifier or surfactant. Preferred compositions of this invention comprise from about 0.5% to about 20%, more preferably from about 1% to about 10%, most preferably from about 1% to about 5%, by weight of the composition, of a structuring agent. The preferred structuring agents of the present invention are selected from the group consisting of stearic acid, palmitic acid, stearyl alcohol, cetyl alcohol, behenyl alcohol, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

A wide variety of anionic surfactants are also useful herein. See, e.g., U.S. Pat. No. 3,929,678, to Laughlin et al., issued Dec. 30, 1975.

The preferred oil-in-water emulsions comprise from about 0.05% to about 10%, preferably from about 1% to about 6%, and more preferably from about 1% to about 3% of at least one hydrophilic surfactant which can disperse the hydrophobic materials in the water phase (percentages by weight of the topical carrier). The surfactant, at a minimum, must be hydrophilic enough to disperse in water. Suitable surfactants include any of a wide variety of known cationic, anionic, zwitterionic, and amphoteric surfactants. See, McCutcheon's. Detergents and Emulsifiers, North American Edition (1986), published by Allured Publishing Corporation; U.S. Pat. No. 5,011,681 to Ciotti et al., issued Apr. 30, 1991; U.S. Pat. No. 4,421,769 to Dixon et al. issued to Dec. 20, 1983; and U.S. Pat. No. 3,755,560. The exact surfactant chosen depends upon the pH of the composition and the other components present. Preferred are cationic surfactants, especially dialkyl quaternary ammonium compounds, examples of which are described in U.S. Pat. No. 5,151,209 to McCall et al. issued to Sep. 29, 1992; U.S. Pat. No. 5,151,210 to Steuri et al., issued to Sep. 29, 1992; U.S. Pat. Nos. 5,120,532; 4,387,090; 3,155,591; 3,929,678; 3,959,461; McCutcheon's, Detergents & Emulsifiers (North American edition 1979) M.C. Publishing Co.; and Schwartz, et al., Surface Active Agents, Their chemistry and Technology, New York: Interscience Publishers, 1949.

Alternatively, other useful cationic emulsifiers include amino-amides. Non-limiting examples of these cationic emulsifiers include stearamidopropyl PG-dimonium chloride phosphate, behenamidopropyl PG dimonium chloride, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof.

The preferred oil-in-water emulsion comprises from about 25% to about 98%, preferably from about 65% to about 95%, more preferably from about 70% to about 90% water by weight of the topical carrier.

The cosmetic composition can be formulated in any of a variety of forms utilized by the cosmetic industry for skin application including solutions, lotions, sprays, creams, ointments, salves, gels, etc., as described herein.

Preferably, the cosmetic composition is formulated viscous enough to remain on the treated skin area, does not readily evaporate, and/or is not easily removed by rinsing with water, but rather is removable with the aid of soaps, cleansers and/or shampoos.

Methods for preparing compositions having such properties are well known to those skilled in the art, and are described in detail in Remington's Pharmaceutical Sciences, 1990 (supra); and Pharmaceutical Dosage Forms and Drug Delivery Systems, 6th ed., Williams & Wilkins (1995).

The topical compositions of some embodiments of the invention, include but are not limited to lotions and creams, may comprise a dermatologically acceptable emollient. Such compositions preferably contain from about 2% to about 50% of the emollient. As used herein, “emollient” refers to a material useful for the prevention or relief of dryness, as well as for the protection of the skin. A wide variety of suitable emollients are known and may be used herein. See, e.g., Sagarin, Cosmetics, Science and Technology, 2nd Edition, Vol. 1, pp. 3243 (1972), which contains numerous examples of materials suitable as an emollient. A preferred emollient is glycerin. Glycerin is preferably used in an amount of from or about 0.001 to or about 20%, more preferably from or about 0.01 to or about 10%, most preferably from or about 0.1 to or about 5%, e.g., 3%.

Lotions and creams according to some embodiments of the invention generally comprise a solution carrier system and one or more emollients. Lotions typically comprise from about 1% to about 20%, preferably from about 5% to about 10% of emollient; from about 50% to about 90%, preferably from about 60% to about 80% water; and a pharmaceutically effective amount of an agent described herein. A cream typically comprises from about 5% to about 50%, preferably from about 10% to about 20% of emollient; from about 45% to about 85%, preferably from about 50% to about 75% water; and a pharmaceutically effective amount of an agent described herein.

The topically applied cosmetic composition of the present invention may also include additional components which are added, for example, in order to enrich the cosmetic compositions with fragrance and skin nutrition factors.

Such components are selected suitable for use on human keratinous tissue without inducing toxicity, incompatibility, instability, allergic response, and the like within the scope of sound medical judgment. In addition, such optional components are useful provided that they do not unacceptably alter the benefits of the active compounds of the invention.

The CTFA Cosmetic Ingredient Handbook, Second Edition (1992) describes a wide variety of non-limiting cosmetic ingredients commonly used in the skin care industry, which are suitable for use in the compositions of the present invention. Examples of these ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyffhizinate), skin treating agents, thickeners, and vitamins and derivatives thereof.

The cosmetic composition can be applied directly to the skin. Alternatively, it can be delivered via normal skin application by various transdermal drug delivery systems which are known in the art, such as transdermal patches that release the composition into the skin in a time released manner. Other drug delivery systems known in the arts include pressurized aerosol bottle, iontophoresis or sonophoresis. Iontophoresis is employed to increase skin permeability and facilitate transdermal delivery. U.S. Pat. Nos. 5,667,487 and 5,658,247 discloses an ionosonic apparatus suitable for the ultrasonic-iontophoretically mediated transport of therapeutic agents across the skin. Alternatively, or in addition, liposomes or micelles may also be employed as a delivery vehicle.

The emollients include, but are not limited to, hydrocarbon oils and waxes, such as mineral oil, petrolatum, and the like, vegetable and animal oils and fats, such as olive oil, palm oil, castor oil, corn oil, soybean oil, and the like, and lanolin and its derivatives, such as lanolin, lanolin oil, lanolin wax, lanolin alcohols, and the like. Other emollients include esters of fatty acids having 10 to 20 carbon atoms, such as including myristic, stearic, isostearic, palmitic, and the like, such as methyl myristate, propyl myristate, butyl myristate, propyl stearate, propyl isostearate, propyl palmitate, and the like. Other emollients include fatty acids having 10 to 20 carbon atoms, including stearic, myristic, lauric, isostearic, palmitic, and the like. Emollients also include fatty alcohols having ten to twenty carbon atoms, such as cetyl, myristyl, lauryl, isostearyl, stearyl and the like.

Although some are water soluble, polyhydric alcohols and polyether derivatives are included as emollients, including glycols, glycerol, sorbitol, polyalkylene glycols and the like, such as propylene glycol, dipropylene glycol, polyethylene glycol 200-500, and the like. The water soluble examples are preferred.

Examples of surfactants include, but are not limited to, spolyoxyalkylene oxide condensation products of hydrophobic alkyl, alkene, or alkyl aromatic functional groups having a free reactive hydrogen available for condensation with hydrophilic alkylene oxide, polyethylene oxide, propylene oxide, butylene oxide, polyethylene oxide or polyethylene glycol Particularly effective are the condensation products of octylphenol with about 7 to about 13 moles of ethylene oxide, sold by the Rohm & Haas Company under their trademark TRITON 100® series products.

Other ingredients such as, fragrances, stabilizing agents, dyes, antimicrobial agents, antibacterial agents, anti-agglomerates, ultraviolet radiation absorbers, and the like are also included in the composition comprising the HIF activator of some embodiments of the invention.

A conditioner agent stable to acid hydrolysis, such as a silicone compound having at least one quaternary ammonium moiety along with an ethoxylated monoquat is preferably also utilized in order to stabilize and optionally thicken the composition with the HIF activator of some embodiments of the invention.

An optional thickener also can be included to improve composition esthetics and facilitate application of the composition to the hair. Nonionic thickeners in an amount of 0% to about 3% by weight are preferred. Exemplary thickeners are methylcellulose, hydroxybutyl methylcellulose, hydroxypropylcellulose, hydroxypropyl methylcellulose, hydroxyethyl ethylcellulose and hydroxyethylcellulose, di (hydrogenated tallow) phthalic acid amide, crosslinked maleic anhydride-methyl vinyl ether copolymer, guar gum, xanthan gum and gum arabic.

The carrier of the conditioning composition is predominantly water, but organic solvents also can be included in order to facilitate manufacturing of the composition or to provide esthetic properties, such as viscosity control. Suitable solvents include the lower alcohols like ethyl alcohol and isopropyl alcohol; glycol ethers, like 2-butoxyethanol, ethylene glycol monoethyl ether, propylene glycol and diethylene glycol monoethyl ether or monomethyl ether; and mixtures thereof. Non-aqueous solvents can be present in the conditioning composition of some embodiments of the invention in an amount of about 1% to about 50%, and in particular about 5% to about 25%, by weight of the total weight of the carrier in the composition.

Non-limiting conditioning agents which may be used in opaque conditioners include: stearyltrimethylammonium chloride; behenetrimethylammonium chloride; cetrimonium bromide; soytrimonium chloride; tallowtrimonium chloride; dihyrogenatedtallowdimethylammonium chloride; behentrimethylammonium methosulfate; Peg-2 Oleammonium chloride; dihyrogenatedtallowdimethylammonium bromide; dihyrogenatedtallowdimethylammonium methosulfate; palmityltrimethylammonium chloride; hydrogenated tallowtrimethylammonium chloride; hydrogenated tallowtrimethylammonium bromide; dicetyidimethylammonium chloride; distearyldimethylammonium chloride; dipalmityidimethylammonium chloride; hydrogenated tallowtrimethylammonium methosulfate; cetrimonium tosylate: eicosyltrimethylammonium chloride, and ditallowdimethylammonium chloride.

Materials that can be used to opacify compositions of the invention include fatty esters, opacifying polymers, such as styrene polymers, like OPACIFIER 653 from Morton, International, Inc.; and fatty alcohols. The following is a non-limiting list of fatty alcohols: cetyl alcohol; stearyl alcohol; cetearyl alcohol; behenyl alcohol; and arachidyl alcohol. Conditioning compositions of the invention which are not clear also can include Lexamine S-13, dicetylammonium chloride, and ceteareth-20.

Cosmetic carriers are well known in the art, e.g., reviewed by Tanya M. Barnes et al., 2021. Pharmaceutics 2021, 13(12), 2012; “Vehicles for Drug Delivery and Cosmetic Moisturizers: Review and Comparison”, which is fully incorporated herein by reference.

Compositions of the present invention may, if desired, be presented in a dispenser device or a kit, along with appropriate instructions for use and labels indicating FDA approval for use in increasing production of collagen, e.g., for tissue regeneration and/or repair.

According to some embodiments of the invention, the tissue regeneration is at a site selected from the group consisting of bone, skin, hair and cartilage.

According to some embodiments of the invention, the tissue regeneration is of the skin.

According to some embodiments of the invention, the administering comprises topically administering.

According to some embodiments of the invention, the method further comprising administering to the subject at least one agent selected from the group consisting of hyaluronic acid (HA) and Botulinum Toxin-Type A (Botox).

Methods of administering the isolated collagen into a skin are known in the art and include, for example, intradermal injections, gels, liquid sprays and patches which comprise the active agent and which are applied on the outer surface of the skin. According to some embodiments of the invention, administration of the isolated collagen into the skin of the subject is performed topically (on the skin).

According to some embodiments of the invention, administration of the isolated collagen into the skin of the subject is performed non-invasively, e.g., using a gel, a liquid spray or a patch comprising the active ingredient, which are applied onto the skin of the subject.

There are two main types of skin patches which can be used to administer the isolated collagen into the skin of a subject. These are the reservoir type patch and the matrix type patch. The reservoir patch usually contains a structure filled with a solid drug (active agent) and a dilute solution, or a highly concentrated drug solution within a polymer matrix and is surrounded by a film or membrane of rate-controlling material. The matrix patch contains a drug and a polymer which form a homogenous system from which the drug is released by diffusion into the external environment. It should be noted that as the release continues, its rate in the matrix type patch usually decreases since the active agent has a progressively longer distance and therefore requires a longer diffusion time to release. For further details and examples of transdermal drug delivery see Prausnitz M R., et al., 2004. Nature Reviews, 3:115-124; Scheindlin S., 2004. Transdermal drug delivery: Past, present, future. Molecular Interventions. Vol. 4:308-312; Prausnitz M R and Langer R., 2008, Nature Biotechnology. 26:1261-1268; Tanner T, and Marks R, 2008, Delivery drugs by transdermal route: review and comment. Skin Research and Technology, 14: 249-260; each of which is hereby incorporated by reference in its entirety.

A non-limiting example of an epicutaneous drug delivery patch, which can be used to administer the isolated collagen into the skin according to the teachings of the invention, is described in Senti G., et al., 2009, J Allergy Clin Immunol. September 4. [Epub ahead of print], which is hereby incorporated by reference in its entirety. According to some embodiments of the invention, administering the isolated collagen to the skin is performed using a reservoir type patch.

According to some embodiments of the invention, administering the isolated collagen to the skin is effected on an intact skin (e.g., a skin which has not been breached, peeled or physically/chemically permeabilized).

For example, administering into an intact skin can be performed using an occlusive patch with semi-solid reservoir and a plastic backing adhesive contour and protective removable cover.

A semi-solid reservoir can be any gel, cream, ointment, emulsion, suspension, microparticles, using various excipients such as fats, oils (e.g., mineral oil, vaselin, vegetable oil or silicon oil), polymers, gelling agent, suspending agent, stabilizers, hydrophilic solvents, Propylene glycol, polyethylene glycols, stabilizing surfactants, colloids etc. and their combinations.

It should be noted that in order to increase delivery of the isolated collagen into the skin, the active agent can be formulated with various vehicles designed to increase delivery to the epidermis or the dermis layers. Such vehicles include, but are not limited to liposomes, dendrimers, noisome, transfersome, microemulsion and solid lipid nanoparticles (for further details see Cevc, G. Transfersomes, liposomes and other lipid suspensions on the skin: permeation enhancement, vesicle penetration, and transdermal drug delivery. Crit. Rev. Ther. Drug Carrier Syst. 13, 257-388 (1996), which is hereby incorporated by reference in its entirety; Kogan A, Garti N. Microemulsions as transdermal drug delivery vehicles. Adv Colloid Interface Sci 2006; 123-126:369-385, which is hereby incorporated by reference in its entirety). In addition, the active agent can be mixed with chemical enhancers such as sulphoxides, azones, glycols, alkanols and terpenes which enhance delivery of active agents into the skin (for further details see Karande P, Jain A, Ergun K, Kispersky V, Mitragotri S. Design principles of chemical penetration enhancers for transdermal drug delivery. Proc Natl Acad Sci USA 2005; 102:4688-4693; Williams A C, Barry B W. Penetration enhancers. Adv Drug Deliv Rev 2004; 56:603-618; and Smith, E W.; Maibach, H I., editors. Boca Raton, FL: Taylor and Francis Group; 2006. Percutaneous Penetration Enhancers; each of which is hereby incorporated by reference in its entirety).

The patch may include the isolated collagen formulated within an emulsion designed to facilitate permeabilization of drugs to the epidermis or the dermis. For example, the patch may comprise the isolated collagen within an oil-in-glycerin emulsion, which is designed to facilitate permeabilization of the isolated collagen through the stratum-corneum and into the dermis. A non-limiting example of an oil-in-glycerin emulsion suitable for delivery through the stratum-corneum into the dermis is described in US Patent Application No. 20040067244, which is hereby incorporated by reference in its entirety. Such an oil-in-glycerin emulsion exhibits a mean droplet size below one micron, and comprises a continuous glycerin phase; at least one vegetable oil comprising an internal phase; at least one emulsifying stabilizer; and at least one bioactive compound comprising at least one hydrophobic, moiety within its structure, wherein the composition facilitates permeabilization of the bioactive compound through the stratum-corneum and into the dermis.

According to some embodiments of the invention, administering the isolated collagen to the skin is effected on a breached skin [e.g., a skin that has been permeabilized (e.g., ruptured) with an external object and the like].

According to some embodiments of the invention, breaching of the skin is effected temporarily (e.g., performed for a pre-determined short period) and is designed to enable better permeabilization of the active ingredient into the skin.

Breaching of the skin can be performed, for example, by introducing micro-holes (e.g., microchannels) in the outer layer of the skin. Such microchannels can be formed using for example, the Radio-Frequency (RF)-Microchannel™ (TransPharma Medical™ Ltd.) technology [Hypertext Transfer Protocol://World Wide Web(dot)transpharma-medical(dot)com/technology_rf(dot)html].

Additionally or alternatively, delivery of the isolated collagen from the patch to the epidermis layer of the skin can be enhanced using physical enhancers known in the art such as ultrasound, ionophoresis, electroporation, magnetophoresis, microneedle and continuous mixing [see e.g., Rizwan M, Aqil M, Talegaonkar S, Azeem A, Sultana Y, Ali A. Enhanced transdermal drug delivery techniques: an extensive review of patents. Recent Pat Drug Deliv Formul. 2009; 3(2):105-24, which is hereby incorporated by reference in its entirety].

According to some embodiments of the invention, administering the isolated collagen is performed by an intradermal injection.

The isolated collagen can be administered into the dermal layer of the skin of the subject by an intradermal injection as described for the Mantoux C (1908) test. Briefly, the isolated collagen can be injected intracutaneously (using for example, a 0.5-ml or 1.0 ml tuberculin syringe through a 26-gauge or 27-gauge needle). The syringe can be placed at an angle of 45 degrees to the skin, and the bevel of the needle is angled downward, facing the skin, and penetrating entirely but not deeper than the superficial layers of the skin. A volume of approximately 0.01 to 0.05 ml (e.g., about 0.02 ml) is gently injected to produce a small superficial bleb (Middleton's Allergy principles&practice, 6th edition 2003).

According to some embodiments of the invention, administering the isolated collagen is performed using a liquid spray (e.g., a spray which includes the isolated collagen in a pre-determined concentration and dosage).

According to some embodiments of the invention, administering the isolated collagen is performed using a gel (e.g., a gel which includes the isolated collagen in a pre-determined concentration and dosage).

For example, for administration using a gel or a spray, a predefined area for administration of the hormone is selected and optionally bounded using an accessory equipment (see e.g., Hypertext Transfer Protocol://World Wide Web(dot)truetest(dot)com).

According to some embodiments of the invention, administering the isolated collagen to the skin is effected such that the required hormone dose is delivered to the skin epidermis and/or dermis layers within a short time, mimicking the effect of an injection into the epidermis or dermis layers.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### General Materials and Experimental Methods

### Materials and Reagents

### Molecules

ML228: ML228 with purity>98% was purchased as powder (Tocris bioscience, Cat #4565; CAS no. 1357171-62-0) and dissolved in Dimethyl sulfoxide (DMSO; Sigma Aldrich), to a stock solution of 50 mM. For experiments described in Examples 1 and 2 below the final DMSO concentration in the cells was 0.04%, for an ML228 concentration of 20 μM. For experiments described in Example 5 below the final DMSO concentration was 0.01%.

Daprodustat: Daprodustat with purity of 98% (MedChemExpress, CAS no. 960539-70-2) was purchased as a powder and dissolved in DMSO to a stock solution of 25 mM. The final DMSO concentration in the cell medium was 0.01%.

Enarodustat: Enarodustat with purity of 97% (TargetMol Chemicals Inc, CAS no. 1262132-81-9) was purchased as a powder and dissolved in DMSO to a stock solution of 50 mM. The final DMSO concentration in the cell medium was 0.01%.

Molidustat: Molidustat with purity of 98% (Adooq Bioscience, CAS no. 1154028-82-6) was purchased as a powder and dissolved in DMSO to a stock solution of 25 mM. The final DMSO concentration in the cell medium was 0.01%.

Vadadustat: Vadadustat with purity of 98% (MedChemExpress, CAS no. 1000025 Jul. 9) was purchased as a powder and dissolved in DMSO to a stock solution of 50 mM. The final DMSO concentration in the cell medium was 0.01%.

Roxadustat: Roxadustat with purity of 95% (Combi-Blocks, Inc., CAS no. 808118-40-3) was purchased as a powder and dissolved in DMSO to a stock solution of 50 mM. The final DMSO concentration in the cell medium was 0.01%.

### Antibodies and Cell Staining

The following antibodies were used: Collagen-I (Abcam cat #ab138492), HIF-1α (Abcam cat #ab179483), β-tubulin (Merck cat #05-661, AP124P), HRP-conjugated secondary antibody Goat Anti-Rabbit IgG (Merck cat #AP156P), Alexa Fluor 488-conjugated goat anti-rabbit IgG (Abcam cat #ab150077), Goat Anti-Mouse IgG H&L (Alexa Fluor 647) (ab150115, Abcam), DAPI Staining Solution (ab228549, Abcam). Cellular viability was assessed using a live/dead viability assay (04511, Merck) and cell-titer (G7570, Promega).

For the experiments described in Example 5 below using various molecules, the following antibodies and cell staining were used: recombinant rabbit anti-mouse Collagen-I antibody [EPR24331-53] (Abcam cat #ab270993), Alexa Fluor 488-conjugated goat anti-rabbit IgG (Abcam cat #ab150077), DAPI Staining Solution (Abcam cat #ab228549) or DRAQ5™ (Abcam cat #ab108410) for nuclei staining and Phalloidin-Atto 594 (51927, Sigma Aldrich) for F-Actin staining. Cell viability was assessed using Alamar blue (Enco, Petach Tikvah, Israel), which includes the non-toxic, cell-permeable compound resazurin, which upon entering living cells, is reduced to resorufin, a compound that is red in color and highly fluorescent.

### Peptides

Table 3 (below) lists some of the peptides used in the experiments.

TABLE 3

| Peptide Name | Peptide Sequence | SEQ ID NO: |
|---|---|---|
| Def16 | GRCRGFRRRCFCTTHC | 1 |
| EPAS1 | ELDLETLAPYIPMDGEDFQL | 2 |
| Def16-EPAS1 | GRCRGFRRRCFCTTHCELDL | 3 |
| | ETLAPYIPMDGEDFQL | |
| HIF | DLDLEALAPYIPADDDFQLR | 4 |

Table 3. Peptide names and sequences are provided. “HIF” is a peptide derived from the HIF1A protein [corresponds to amino acids 556-575 of HIF1A GenBank Accession No. NP_001521.1 (SEQ ID NO:5)]; “EPAS1” is a peptide derived from the HIF2A (EPAS1) protein [corresponds to amino acids 523-542 of EPAS1 GenBank Accession No. NP_001421.2 (SEQ ID NO: 6)]; “Def16” is a peptide derived from the defensin (chain A) protein [corresponds to amino acids 32-47 of the defensin (chain A) protein GenBank Accession No. 2LR3_A (SEQ ID NO: 7)].

### Cell Cultures

### Human Fibroblasts

Human fibroblasts were isolated from fragments of healthy masticatory oral mucosa (from the posterior hard palate) collected during periodontal surgeries. Informed consent was obtained from all patients. Shallow tissue samples (without submucosa) with a diameter of approximately 2 mm were excised using a #15C scalpel blade. Exclusion criteria for potential tissue donors were smoking, any systemic disease, pregnancy or lactation, or a history of periodontal disease. Cells were separated into connective tissue and epithelium using dispase II (Sigma-Aldrich, Rehovot, Israel) at 2 mg/mL for two hours at 37° C. Subsequently, the connective tissue was cut into smaller pieces, which were then placed in a standard culture medium (Dulbecco's Modified Eagle Medium (DMEM) supplemented with 10% fetal calf serum (FCS) and 2 mM glutamine, 100 U/mL penicillin, 100 mg/mL streptomycin, 12.5 U/mL nystatin, 0.11 mg/mL sodium pyruvate, and non-essential amino acids at 37° C. in 5% CO₂. Before treatment with ML228 40,000 cells/well were seeded in a 96 wells-plate. Cell viability was assessed manually by trypan blue dye exclusion (Biological Industries). First to third passage cells were used only if they had a typical fibroblastic morphology and a cell viability level>95%.

### Human Osteoblasts

MG-63 human osteosarcoma cell lines were obtained from the American Type Culture Collection (ATCC, Manassas, VA, USA). Cells were cultured in α-MEM supplemented with 10% FCS, 100 U/ml penicillin, and 100 mg/ml streptomycin at 37° C. in a humidified atmosphere incubator containing 5% CO₂. Before treatment with ML228, 5,000 and 50,000 cells/well were seeded in a 96 or 24 well-plate, respectively.

### Confocal Imaging

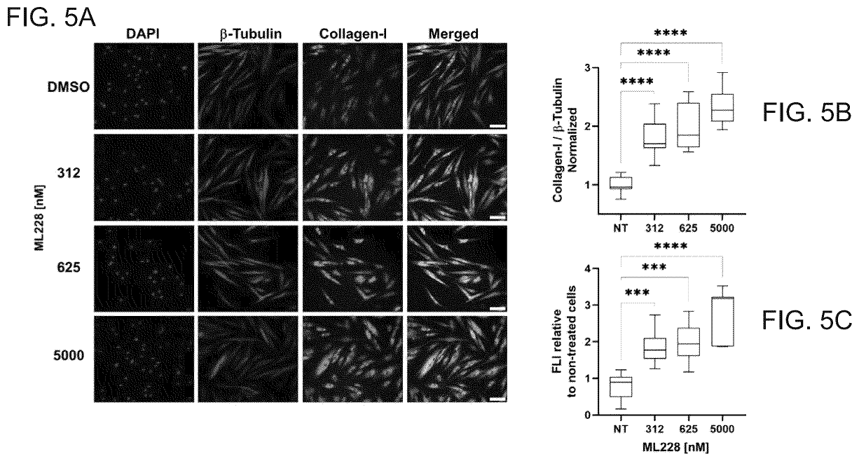

Cells were cultured to sub-confluence on a 96-well plate culture plate or on round-glass cover slips in a 24 well plate and incubated with ML228 at 37° C. in 5% CO₂ for 24-96 hours. Cells were fixed for 20 minutes with 4% paraformaldehyde in phosphate-buffered saline (PBS), washed 3 times with PBS, blocked with 2% bovine serum albumin diluted in PBS, which contains 0.1% Triton X-100 for 20 minutes. Fixed cells were incubated with 1:200 of rabbit anti-human type I collagen (Abcam, Cambridge, UK) for 1 hour at 25° C. After washing with PBS, cells were incubated with 1:1000 of Alexa Fluor 488-conjugated goat anti-rabbit IgG (Abcam, Cambirdge, UK) and Goat Anti-Mouse IgG H&L (Alexa Fluor 647). Cells nuclei were stained with 1:1000 4′,6-diamidino-2-phenylindole (DAPI, final concentration of 1 mg/ml) for 1 hour. Cell images were taken using a ZEISS LSM 900, Laser Scanning Microscope, using Zen 3.1. Fluorescence readouts were obtained with excitation/emission (ex/em) of 495/519 nanometer (nm; green) and 340/488 nm (blue) (for FIGS. 2B and 2D); or with ex/em of 358/461 nm (blue), 495/519 nm (green) and 652/668 nm (red) (for FIG. 5A).

### Cell Viability and Live/Dead Assay

The live/dead viability assay (cat #04511-1KT-F Merck, Germany) was used according to the manufacturer's protocol. Briefly, cells were incubated in a medium supplemented with the indicated concentrations of ML228 for 48 hours. Fluorescein diacetate (6.6 μg/mL) and propidium iodide (5 μg/mL) (Merck, NJ, USA) were prepared in DMEM and added to the cells. Confocal images were captured after addition of the dyes with ZEISS LSM 900, Laser Scanning Microscope. Fluorescence readouts were obtained with ex/em of 490/515 nm and 535/617 nm for green and red, respectively. Cellular ATP levels were quantified using the cell-titer assay (Promega, WI, USA cat #G7570).

### Image Analysis

All images were analyzed with the computer-based image analysis system (Image J) using the Fiji 2 software (National Institute of Health). Equal field areas were selected for analysis and quantification of fluorescence signals. Quantification of fluorescence signal intensity measured by color threshold and normalized against β-tubulin intensity. Total collagen was evaluated by total fluorescence area intensity.

### Western Blot Analysis

Cells at a density of 50,000 cells/well in a 24-well plate were cultured for 2 days with variable ML228 concentrations. Cells were sonicated and lysed in a buffer containing 20 mM Tris-HCL pH=7.5 with a protease inhibitor (Abcam, Cambridge, UK) or a protease inhibitor from Merck (539134, Merck). Equal amounts (1 μg) of total protein were loaded and tested using Western blotting. Samples were divided for testing native samples and denaturative samples, which were dependent on each antibody recognition sites. Samples size was separated by a 6% sodium dodecyl sulfate (SDS) gel and transferred to nitrocellulose membranes that was further incubated overnight with primary antibodies of HIF-1α, and β-tubulin. As for detecting Collagen-I, samples were prepared in native conditions absent of SDS and β-mercaptoethanol. Thereafter, samples were separated by a native gel. Detection of Collagen-I was done in 6% native gel enabling observation of non-denatured structural collagen and preventing thermal degradation of the heat sensitive collagen. After extensive washing with TBST (Tris-buffered saline supplemented with 0.1% Tween-20), the membrane was further incubated with an HRP-conjugated secondary antibody for 1 hour. Chemiluminescence values were read after the addition of ECL substrate in a ChemiDoc imager (Biorad CA, USA).

### Osteogenic Differentiation of MG-63 Cells

The cultivation of MG-63 was performed in α-MEM growth medium (Sartorius). Osteogenic differentiation was performed by plating 20,000 cells/well into a sterile 24-well plate. Cells were left to adhere for 1 day and then switched to the osteogenic medium consisting of a minimum essential medium alpha (α-MEM) supplemented with 10% heat-inactivated FBS, 2 mM L-glutamine, 100 U/mL penicillin, 100 μg/mL streptomycin, 50 μM ascorbate-2-phosphate, and 10 mM β-glycerophosphate. Medium was changed every 2-3 days together with peptide supplementation. Seven to twenty-eight days after induction of differentiation, the production of extracellular calcified mineral matrix was visualized by Alizarin Red staining (Sigma) and Picrosirius Red (Scytek).

### Alizarin-Red Staining for Visualization of Mineralization

To visualize calcium deposition upon osteogenic differentiation, an Alizarin-red-S (Sigma) stock solution of 40 mM was used. Cells were washed three times with PBS and fixed in 1 mL of 4% PFA for 20 minutes at room temperature (RT). After fixation, cells were rinsed with double distilled water (DDW) and stained for 10 minutes in 1 mL Alizarin-red-S stock solution under continuous rotation. To diminish unspecific staining, cells were washed extensively three times with PBS. Stained samples were stored at 4° C. covered with PBS.

### Picrosirius Red Staining

To visualize the extent of ECM formation, Picrosirius red dye was used to stain Collagen-I and Collagen-III. Treated cells were fixed with 4% PFA, washed with PBS and dried at 37° C. or at room temperature (RT) for overnight incubation. Then, the cells were stained with 400 μL of Sirius Red dye (0.1% in saturated picric acid) for 1 hour with mild shaking. The stained cell layers were then extensively washed with 0.01 N HCl to remove all non-bounded dye. After rinsing, photographs were taken under confocal microscope using polarized light.

### Cell Staining and Immunofluorescence Analysis Following Treatment With a Peptide

For microscopy images, cells were cultured in a 24-well plate and incubated with medium supplemented with the indicated concentrations of the treating entity (e.g., peptides or DFO) at 37° C. under 5% CO₂. Cells were then washed additional three times with cold phosphate-buffered saline (PBS), following a 20 minute fixation in a 4% paraformaldehyde (PFA) in PBS and extensively washed three times with PBS to remove the PFA, followed by a 20 minute wash with blocking and penetration solution (PBS with 0.1% Triton X-100 and 2% BSA). Fixed cells were incubated with antibodies for 1 hour at 25° C., washed, and developed together with fluorescence-tagged secondary antibodies (Alexa 488 and 647, Abcam). Cell nuclei were stained with 1 mg/mL 4′,6-diamidino-2-phenylindole (DAPI Staining Solution ab228549, Abcam). Images were taken using a ZEISS LSM 900 in confocal mode, using Zen 3.1. Fluorescence readouts were obtained at ex/em of 358/461, 495/519 nm, and 652/668 nm for blue, green, and red, respectively.

### Quantitative Binding Assay

Generation of PHD polypeptide—For binding assays between PHD and the peptide, the PHD2/EGLN1 (SEQ ID NO: 27) was expressed in PGEX and pET plasmids as a cleavable GST fusion protein in E. coli BL21 cells. Growing in LB, after reaching to OD_{(600 nm)}=0.8, protein expression was induced by the addition of 1 mM IPTG at 25° C. The cells were harvested after 16 hours and resuspended in PBS-based lysis buffer suitable for downstream purification onto GST column (Glutathione Sepharose 4 Fast Flow) of the soluble fraction after disrupted by sonication and remove of all non-soluble debris by centrifuge. Elution fraction from the GST column was collected for further analysis. Purification was performed using SDS-PAGE and Coomassie stain.

Fluorescence polarization (FP) assay—Fluorescence Polarization (FP) measurements were performed on samples arrayed in a 96-well plate using Biotek HybridH1 reader equipped with a polarized optic system. Binding assay was evaluated by adding variable concentrations of PHD protein into 200 nM of FITC-labeled peptides. Experimental polarization data were fitted using GraphPad Prism9 into a single site binding model, with error bars representing standard deviation.

### Statistical Analysis

Statistical comparisons were performed by an ordinary one-way ANOVA as implemented in GraphPad Prism with Dunnett post-hoc statistical hypothesis. Each measurement was analyzed against the non-treated sample. For statistical analysis, significance was set as *=0.01≤p<0.05; **=p<0.01; ***=p<0.001; ****=p<0.0001.

### Experiments Using Various Small Molecules

### Cell Culture and Treatment With Small Molecules

Embryonic mouse fibroblast NIH/3T3 cell line (ATCC) were seeded at a concentration of 50*10³ cells/well on 96 well glass bottom plate (P96-1.5H-N, Cellvis) for the immunofluorescence experiment and on cell culture microplate, 96 well, PS, F-Bottom (655090, greiner) for the viability experiment. The cells were cultured with Dulbecco's modified Eagle's medium (DMEM) supplemented with 10% fetal calf serum, 1% Penicillin-Streptomycin solution and 1% L-glutamine (All purchased from Rhenium). At 24 hours post seeding, cells were treated for another 48 hours by replacing them with the cell medium supplemented with a serial dilution (5-0.02 μM, factor dilution of 2) of the ML228, Daprodustat, Enarodustat, Molidustat, Vadadustat or Roxadustat molecules dissolved in DMSO. The final DMSO concentration in the cell medium was 0.01%. Cells were maintained in an incubator at 37° C. in a humidified atmosphere containing 5% CO₂.

### Immunofluorescence and Cell Staining Following Treatment With Small Molecules

At 48 hours post treatment with either Daprodustat, Enarodustat, Molidustat, Vadadustat or Roxadustat, cells were fixed for 10 minutes together with their media with 8% paraformaldehyde (PFA, Electron Microscopy Sciences) dissolved in phosphate-buffered saline (PBS, Invitrogen) to a final concentration of 4% PFA, without any wash to avoid cells detachment from the glass. Cells were briefly washed four times with ice-cold PBS to remove the PFA and kept overnight at 4° C. The following day, a blocking solution which contains 1.5% Bovine Serum Albumin (Sigma Aldrich) dissolved in PBS was applied for 30 minutes, followed by an overnight incubation at 4° C. with recombinant rabbit anti-mouse Collagen-I 1° antibody (primary Ab) [EPR24331-53] (ab270993, Abcam), dissolved in the blocking solution (1:1000 dilution). The following day, cells were washed for 3 times with PBS (5 minutes each), followed by immunostaining with goat anti-rabbit IgG H&L (Alexa Fluor® 488) 2° antibody (secondary Ab) (ab150077, Abcam) dissolved in blocking solution (1:1000 dilution). Cells were washed again for 3 times with PBS (5 minutes each), stained for 30 minutes with DRAQ5™ (ab108410, Abcam) for nuclei staining dissolved in PBS (1:1000 dilution), and washed once again with PBS. For the ML228 experiment, cells were washed with PBS before applying 4% PFA dissolved in PBS. Then, fixed cells were additionally permeabilized with 0.2% Triton™ X-100 (Merck) dissolved in PBS, additionally stained with Phalloidin-Atto 594 (51927, Sigma Aldrich) for F-Actin staining for 1 hour (1:250 dilution) together with the aforementioned secondary antibody, followed by 10 minutes incubation with DAPI staining solution (ab228549, Abcam) for nuclei staining dissolved in PBS (1:1000 dilution).

### Image Acquisition Using the Laser Scan Microscope for Experiments Using Various Small Molecules

Images were taken using a ZEISS LSM 900 in wide-field or in the LSM confocal detection mode (ML228 experiment), using Zen 3.1. Fluorescence readouts were obtained at excitation/emission: 353/465 nm (DAPI), 493/517 nm (Collagen-I), 590/618 (F-Actin) and 647/683 nm (DRAQ5) using a Plan-Apochromat 20×/0.8 M27 objective.

### Cell Viability for Experiments Using Various Small Molecules

For the ML228 experiment, at 48 hours post treatment, cells were stained by replacing the cell medium with Alamar blue (Enco, Petach Tikvah, Israel) diluted 1:10 in the cell medium, and incubated for 4 hours (37° C., 5% CO2). Then, fluorescence readouts were obtained using the Spark multimode microplate reader (Tecan) at excitation/emission 540±20 nm/590±20 nm. Cell viability was calculated as the percentage difference of treated cells with ML228 relative to the control cells at 590±20 nm emission.

### Image Analysis Using Incucyte SX5 for Experiments Using Various Small Molecules

Image acquisition and quantification were performed by Incucyte SX5 (Sartorius) using software version 2022B Rev2. A standard scan was done for 9 images per well using 20× objective, 2 optical modules (green at excitation/emission of 453-485 nm/494-533 nm in 300 msec acquisition time, and near-IR at excitation/emission of 648-674 nm/685-756 nm in 400 msec acquisition time, including Phase channel) following immunofluorescence protocol. For the image analysis, 2-6 replicates per condition were used. Using the Basic Analyzer mode, “Phase Object Confluence (%)” was acquired using the Phase channel. This measurement is the percentage of the image area occupied by cells. “Total Collagen-I Integrated Intensity Per Image (GCU×μm²/Image)” was acquired using the green optical module. GCU stands for Green calibrated Unit, while this measurement is the total sum of the Collagen-I objects' fluorescent intensity in the image. “#Cells (Per image)” was acquired by the near-IR optical module. This measurement was calculated by counting the number of nuclei per image.

### Statistical Analysis for Experiments Using Various Small Molecules

Multiple comparisons were performed by ordinary one-way ANOVA followed by Dunnett's post-hoc statistical hypothesis for comparing each condition mean to the control mean. Statistical analysis was done using GraphPad Prism version 10.0.0 for Windows, GraphPad Software, Boston, Massachusetts USA, worldwideweb(dot)graphpad(dot)com. For statistical analysis, significance was set as (*) p≤0.05; (**) p≤0.01; (***) p≤0.001; (****) p≤0.0001 and (ns; not significant) p>0.05.

### Example 1

### Effect of a HIF-1 Activator on Collagen Levels in Fibroblasts and Osteoblasts

The effect of ML228 (chemical structure thereof is depicted in FIG. 1), a known activator of HIF, on fibroblasts and osteoblasts was analyzed. Fibroblast and osteoblast cells were incubated with variable concentrations of ML228 for 48 hours. Collagen-I levels were evaluated by immunohistochemistry via Collagen-I antibody and a fluorescently-labeled secondary antibody. Cell nuclei were stained with 4,6-diamidino-2-phenylindole (DAPI).

### Experimental Results

The effect of a HIF activator on Collagen-I levels in fibroblasts—Collagen-I levels were determined by immunofluorescence. As shown in FIGS. 2A and 5B-C, there was a 2-3 fold increase in Collagen-I levels in the presence of ML228 when compared to non-treated cells (FIG. 2A, showing absolute mean fluorescence intensity (FLI), without normalization to cell number); and FIG. 5C showing FLI normalized to cell number by DAPI staining), and when compared to the level of β-tubulin under the same treatment conditions (FIG. 5B).

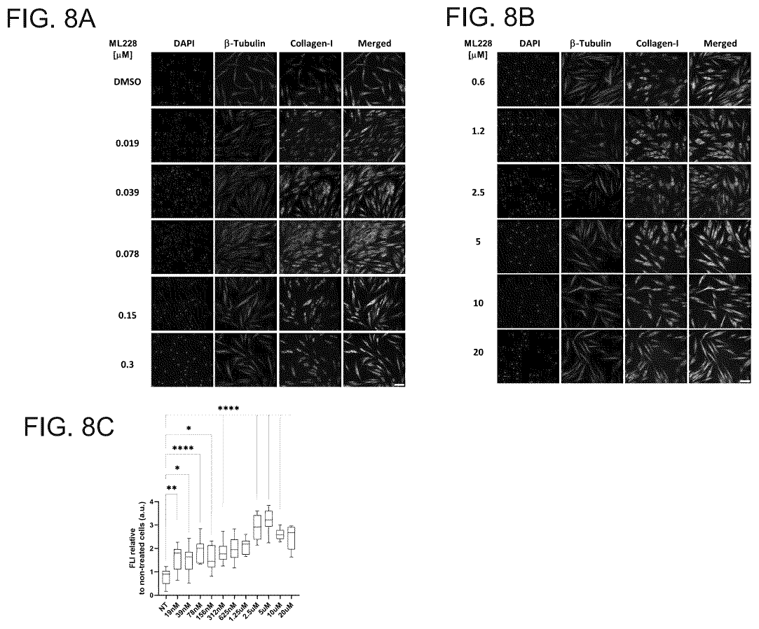

FIG. 5C shows that after treatment with ML228, already at a concentration above 312 nM Collagen-I levels were more than two-fold higher comparing to the baseline of non-treated cells, with maximum collagen levels observed at a concentration of 5 μM. FIGS. 8A-C show that at higher concentrations of ML228, e.g., at concentrations above 10 μM (e.g., 10 or 20 μM of ML228), Collagen-I levels gradually decrease.

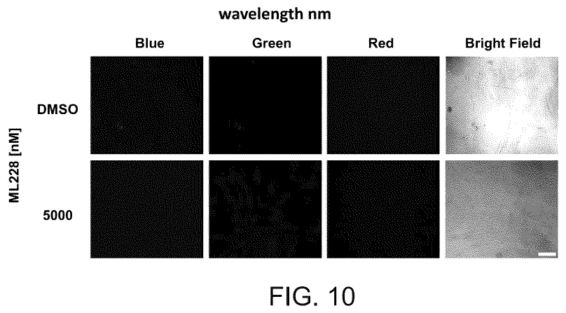

To rule out that ML228 imparts any auto-fluorescence, images were taken from the same treated cells, yet in the absence of any fluorescent antibody (primary and secondary). As is shown in FIG. 10, blue, green and red images taken with the same parameters as described in FIG. 5A but without fluorescence antibodies, demonstrating that ML228 does not impart any detectable autofluorescence.

### The Effect of a HIF Activator on Collagen-I Levels in Osteoblasts

In a similar approach the present inventors have evaluated the effect of ML228 on MG-63 osteoblast-like cells. FIG. 2D shows representative images that visualize the accumulation of Collagen-I in the osteoblast cells in the presence of increasing concentrations of ML228, and FIG. 9A further shows the expression of Collagen-I, β-tubulin and DAPI (green, red and blue, respectively) in the osteoblast cells following treatment with ML228. FIG. 2C shows a bar chart of the absolute mean fluorescence intensity of osteoblast that were stained for Collagen-I without normalization to cell number. FIG. 9B shows the mean fluorescence intensity of osteoblasts that were stained for Collagen-I which was normalized to cell number by DAPI nuclear staining. As can be seen, the maximum enhancement of Collagen-I is in the presence of 5 μM ML228 (FIG. 9B).

### Example 2

### Effect of a HIF-1 Activator on Cell Viability and Proliferation of Fibroblasts and Osteoblasts

### Experimental Results

The effect of ML228 on cellular viability and proliferation of fibroblasts and osteoblasts—To better understand the pattern of Collagen accumulation following treatment with ML228, the present inventors have further studied its effect on cellular viability and cell proliferation.

Effect on proliferation—FIG. 3A shows luminescence-based readout of ATP levels of fibroblasts and osteoblasts following their incubation with variable ML228 concentrations relative to non-treated cells. An IC₅₀ of 567 and 679 nM was measured for the osteoblasts and fibroblasts, respectively. The IC₅₀ values are in congruent to the levels of Collagen-I suggesting that either molecular toxicity or the inability of the cells to proliferate above these specific threshold concentrations hampers additional collagen accumulation. To differentiate between these possibilities, the present inventors examined the mechanism by which ML228 affects fibroblast viability.

Effect on cell viability and death—The present inventors have applied the live/dead assay, which yields distinct green and red colors for live and dead cells, respectively.

FIGS. 3B and 3C show representative images of fibroblast (FIG. 3B) and osteoblast (FIG. 3C) cells that were treated with 0 μM, 0.62 μM and 5 μM of ML228. FIG. 9C shows representative confocal images of MG-63 cells treated with representative concentrations of ML228 (0 μM, 0.62 μM and 5 μM). Almost no dead cells were observed. As a reference for dead cells, images following treatment with 70% ethanol are shown. The fluorescence images (merged in green and red) after incubation of the cells with ML228 for 48 hours show that ML228 negatively regulates cell growth but without imparting cellular death. Quantification of the live/dead assays are shown in FIGS. 3D and 3F (for fibroblasts) and in FIG. 3E (for osteoblasts).

Indeed, the slightly lower IC₅₀ value of cellular proliferation of the osteoblast cells is correlated with the lower concentration of ML228 in osteoblasts that imparts the maximal level of Collagen-I.

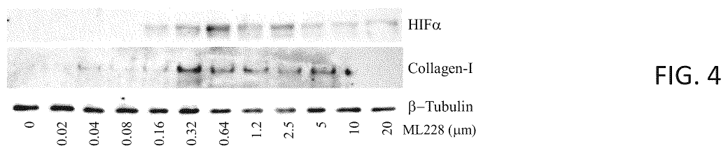

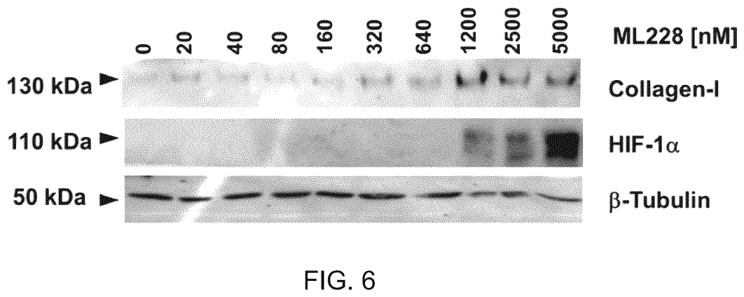

ML228 affects the levels of HIF-1α—Having established that ML228 induces over-accumulation of Collagen-I and does not impart cellular toxicity, the present inventors have further validated that ML228 is indeed correlated with higher levels of HIF-1α. FIGS. 4 and 6 show representative Western blot analyses for HIF-1α, Collagen-I and β-tubulin levels following treatment of fibroblast cells with variable concentrations of ML228. In non-treated cells and in cells treated with low doses of ML228, no HIF-1α protein is observed, indicating that HIF-1α is in its basal levels owing to its fast degradation by the VHL complex. In contrast, in the presence of increasing concentrations of ML228 higher levels of HIF-1α and Collagen-I are shown (FIG. 6). This behavior is correlated with the highest levels of Collagen-I observed in the confocal images (FIG. 5B).

Of note, a major difference between the Western Blot analysis and the immunofluorescence analysis shown in FIGS. 5B and 5C is that Western Blot was evaluated on total cellular lysate including intracellular content.

### Analysis and Discussion

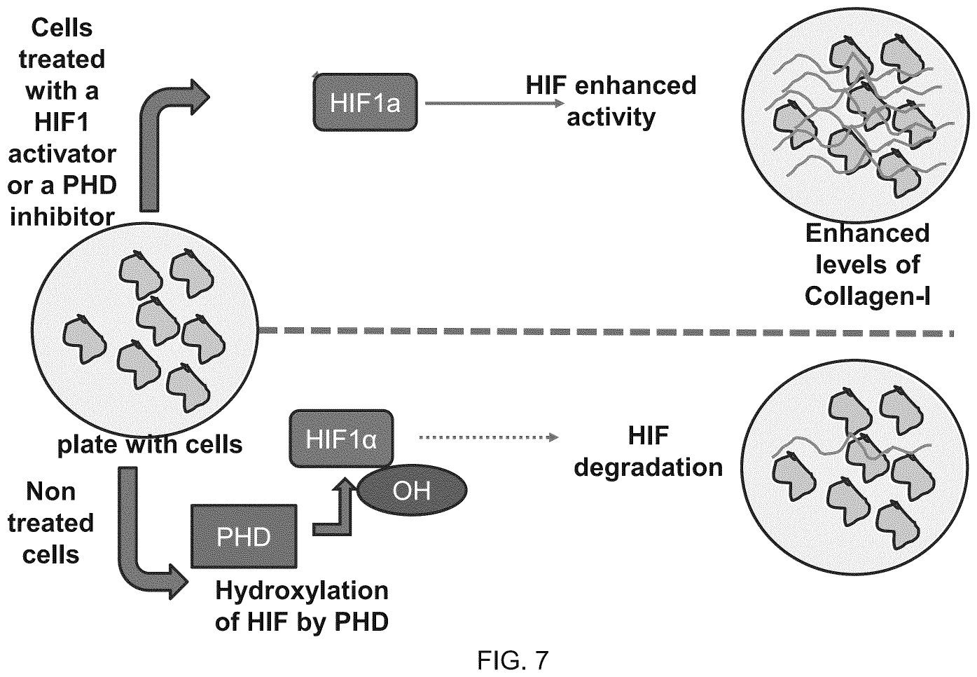

The present inventors have harnessed the hypoxia pathway and showed that modulation of HIF-1α activity by the small molecule ML228 leads to a substantial increase in Collagen-I levels in cells such as fibroblast and osteoblast cells. The present inventors showed that imparting an artificial hypoxia-like state, which enhances HIF-1α activity, increases Collagen accumulation relative to untreated cells. FIG. 7 illustrates the experimental scheme and mechanism by which the application of ML228 activates hypoxia pathway, leading to HIF nuclear translocation and resulting in enhanced Collagen accumulation.

Notably, the ML228 mechanism is not clearly defined and the PHD enzyme may not be the direct target of the molecule. In addition, the cellular viability of ML228-treated cells showed an IC₅₀ of about 600 nM. Without being bound by any theory this relatively high value raises the possibility that Collagen accumulation may be limited by off-site effects that prevent cellular proliferation. Additionally or alternatively, and without being bound by any theory, this suggests that the maximal Collagen level reported here may not be an inherent characteristic of the hypoxia cellular pathway. Accordingly, the design of more specific and potent molecules, and the exploration of additional inhibitors of proteins that regulate HIF, may yield even higher Collagen-I levels.

Although type-I is the major Collagen type used in food biotechnology applications, understanding the effect of activating the hypoxia pathway on additional Collagen types (e.g., type II, III, etc.) is important. As shown in FIGS. 9A-C, Collagen levels increased also in osteoblast MG-63 cells that were treated with ML228. As the HIF pathway is highly conserved, similar patterns may be expected in other cellular systems. Such exploration could lead to a more potent cellular system for producing mammalian Collagen and other ECM factors that are required for food and agricultural applications. In addition to interference with the hypoxia pathway, synergy with other approaches should be explored. An example of this approach is the addition of ascorbic acid (i.e., vitamin C), which is known to stimulate the biosynthesis of procollagen or glycolic acid. Such mixtures could be optimized to further boost Collagen levels. An alternative solution, aimed to increase the activity of HIF-1α by interfering with its interaction with PHD, is to harness proteolysis targeting chimeric (PROTAC) modulators, which impart fast degradation of a target protein. Such a possibility could facilitate PHD degradation and thus maintain high levels of HIF. Finally, several approaches can be synergistically integrated to further increase Collagen levels in mammalian cells. This study demonstrated that modulation of a specific cellular pathway can enhance Collagen production in cells. Indeed, the ML228 molecule, which is designed as an HIF-1α activator, was found by the present inventors to boost Collagen accumulation in fibroblast and osteoblast cells. Due to the essential role of Collagen in various health and disease conditions, enhancing its accumulation has immense implications, including in a broad array of biotechnological applications and in the production of animal-free Collagen for cultured meat.

### Example 3

### Identifying Various Peptides for Interfering With PHD and HIF Interaction and Increasing Collagen Levels

### Experimental Results

Quantitative binding assay—The present inventors have conducted experimental measurements to quantify the binding affinity of the designed peptides to the PHD protein (PHD2, SEQ ID NO: 9), using the Fluorescence Polarization (FP) assay (FIG. 13). The EPAS1 peptide exhibited a Kd value of 4 μM, demonstrating a slight improvement compared to the HIF1 peptide with a Kd value of 7 μM. These results demonstrate that both peptides directly bind PHD enzyme.

Evaluation of Collagen fibers in MG-63 cells upon Def16-EPAS1 treatment—To evaluate the variation in Collagen fiber levels between normal MG-63 cells and MG-63 cells treated with the Def16-EPAS1 peptide, cells were supplemented to a 7-day incubation period with osteogenic differentiation medium (which includes 50 μg/mL ascorbic acid and 10 mM β-glycerophosphate), along with the addition of Def16-EPAS1 peptide. Collagen fibers were highlighted/stained by Picro-Sirius Red staining according to the manufacturer's instructions (FIG. 12). The PSR staining allows to clearly distinguish between Collagen-I and III fibers in cell culture. In the evaluation of polarizing colors of collagens fibers, Collagen-I and III were polarized with 488 nm and 647 nm wavelength, respectively, and visualized with the confocal microscope. As shown in FIG. 12, the addition of the Def16-EPAS1 peptide resulted in over-production of Collagen-I and III.

Def16-EPAS1 induces mineralization in MG-63—Calcium deposition is an important indicator of the activity of bone-forming cells which form a mineralized matrix. Alizarin Red S (ARS) staining, which can be visualized using transmitted light and polarized light, was used to stain MG-63 cells cultured for 7 days in the presence of an osteogenic differentiation medium (which includes 50 μg/mL ascorbic acid and 10 mM β-glycerophosphate) with or without (w/o) the Def16-EPAS1 peptide. The Def16-EPAS1 peptide includes a cell penetrating sequence of 16 amino acids from defensin protein and a 20 amino acid sequence from the EPAS1 protein, and the EPAS1 peptide was shown to efficiently bind PHD (FIG. 13). No calcium deposits were observed in cells cultured with only the osteogenic differentiation medium (FIG. 11, left panels). However, culturing the cells in the presence of the osteogenic differentiation medium with the supplementation of 5 μM or 20 μM of the Def16-EPAS1 peptide resulted in a strong induction of deposition of calcium in MG-63 cells (FIG. 11, middle and right panels).

Effect of Def16-EPAS1 on extracellular matrix (ECM) mineralization in MG-63 cells—To initiate osteogenic differentiation, MG-63 cells were cultured in the presence of a differentiation medium (50 μg/mL ascorbic acid and 10 mM β-glycerophosphate) supplemented with 10 μM of Def16-EPAS1 peptide for 7, 14, 21 and 28 days as indicated in FIG. 15. Then cells were fixed and stained with Alizarin Red S (ARS) and Picrosirius Red. The results using these stains indicate the ability of pre-osteoblast cells to differentiate and form a mineralizing matrix as mature functional osteoblasts (FIG. 15).

Effect of Def16-EPAS1 on Collagen accumulation in Bovine Dermal Fibroblast—To test HIF mimicking peptide and its effect on Collagen accumulation in additional mammalian cells, the present inventors have examined the effect of the Def16-EPAS1 peptide on Bovine Dermal Fibroblast. The present inventors have cultured the bovine Dermal Fibroblast cells with Def16-EPAS1 in a dose dependent manner for 48 hours. FIGS. 16A-B show that after treatment with the Def16-EPAS1 peptide, already at a concentration above (10 μM), Collagen-I levels were more than 2-fold higher as compared to the baseline of nontreated cells. Moreover, thick fibril bundles of Collagen were observed in higher concentrations.

Taken together, these results demonstrate that HIF mimicking peptides stimulated Collagen accumulation. Together with osteogenic factors, the peptide Def16-EPAS1 was responsible for ECM remodeling by enhancing matrix mineralization and overaccumulation of collagen fibers.

Thus, supplementing cells with peptides which mimic the N-terminal transactivation domain (NTAD) of HIF-1 shows an increase in cellular calcification and Collagen accumulation.

This approach of culturing cells with a mimicking peptide results in hypoxia-like effects, has potential therapeutic implications for conditions such as ischemic diseases, tissue regeneration, and certain cancers that rely on HIF-mediated pathways for growth and survival.

### Example 4

### Inhibition of Prolyl Hydroxylation of HIF-1-Alpha Using an Iron Chelator Results in Increased Collagen-I Levels

### Experimental Results

Effect of an iron chelator on Collagen-I levels in MG-63 cells—Iron chelators have been shown to inhibit the activity of PHD enzymes by limiting the availability of iron, which is an essential cofactor for the PHD catalytic activity. The present inventors have incubated MG-63 cells with deferoxamine mesylate (DFO), an iron chelator, to explore the possibility of enhancing Collagen-I levels in these cells. As shown in FIGS. 14A-B, the iron chelator DFO increased Collagen-I production in MG-63 cells, even in the presence of low concentrations of DFO such as 0.15 μM, with maximal levels in the presence of 10-20 μM DFO. FIG. 14A shows representative confocal images that visualize DNA, β-tubulin, and Collagen-I in MG-63 cells. FIG. 14B shows the corresponding bar plot of the mean fluorescence intensity (FLI) of total Collagen-I normalized to β-tubulin as a function of iron chelator concentration. These results show increased accumulation of Collagen-I with increasing concentrations of the iron chelator.

### Example 5

### Small Molecules Which Inhibit HIF-PHD Activity Can Increase Collagen Production

### Experimental Results

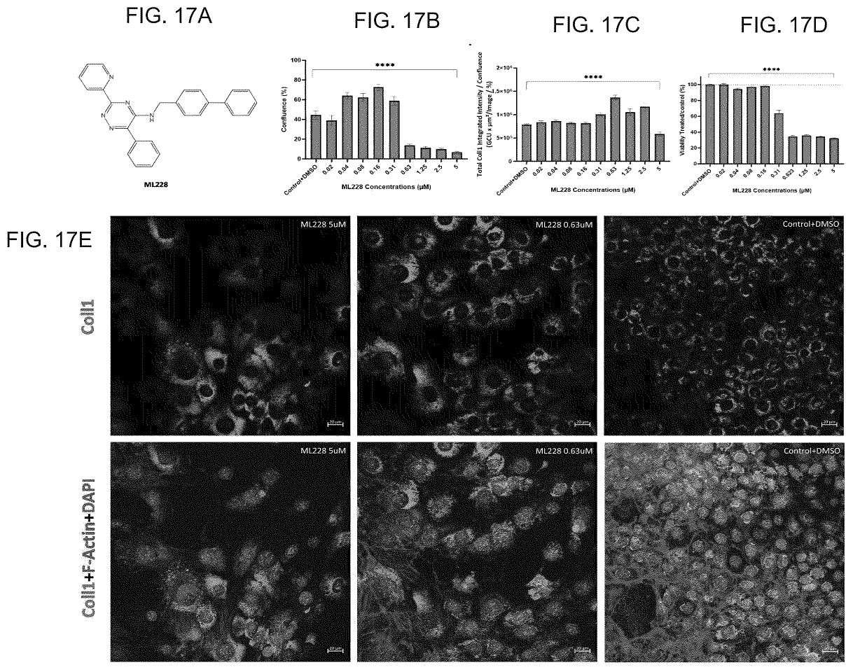

The HIF-1α activator, ML228, increase Collagen type-1 production in Embryonic Mouse Fibroblast NIH/3T3 cell line—The present inventors have further tested the effect of ML228 on Embryonic Mouse Fibroblast NIH/3T3 cell line, which are known for Collagen type-1 production (Karsenty and Park 1995). NIH/3T3 cells were cultured for 48 hours with variable concentrations of ML228 and then were labeled for Collagen type 1 (as described in the GENERAL MATERIALS AND EXPERIMENTAL METHODS section above). Image analysis was used to measure confluence of cells (FIG. 17B). Multiple comparisons were performed by ordinary one-way ANOVA followed by Dunnett's post-hoc statistical hypothesis, show a significant reduction in cell confluence for cells treated with 0.63-5 μM ML228 (p<0.0001), suggesting molecule toxicity for these concentrations. The present inventors have further validated it by an Alamar blue-based viability assay, showing that indeed there is a lower viability for cells treated with 0.31-5 μM ML228 compared to untreated cells (p<0.0001; FIG. 17D). Next, the present inventors have measured the amount of Collagen type 1 (Coll1 for short) normalized to cells confluence using immunofluorescence (FIG. 17C), showing a significant increase for cells treated with 0.31-2.5 μM ML228 cells (0.31 μM; p<0.05, 0.63 μM; p<0.0001, 1.25 μM; p<0.001, 2.5 μM; p<0.0001) and a reduction for cells treated with 5 μM (p<0.05) compared to untreated cells. Representative images of immunofluorescence of NIH/3T3 cells permeabilized, while intracellular Collagen labeling was demonstrated (FIG. 17E), suggesting Pro-collagen type 1 was immunostained.

### Testing the Ability of Inhibitors of HIF-Prolyl Hydroxylases (PHDs) Enzymes to Increase Collagen Type-1 in the NIH/3T3 Cell Line

The present inventors have further aimed to investigate the effect of small molecules inhibition on HIF-prolyl hydroxylases (PHDs) enzymes by applying PHD inhibitors used as competitive inhibitors of 2-oxoglutarate (2-OG), one of the substrates for the enzymatic reaction of PHD. In order for PHD to hydroxylate proline or asparagine residues on HIF-1 alpha, the enzymatic reaction requires molecular oxygen, 2-OG, Fe²⁺, and ascorbic acid as substrates (Hirota and Semenza 2005; Schofield and Ratcliffe 2004; Wilkins et al. 2016). Using these 2-OG analogous which are all active site-targeted inhibitors, the binding of 2-OG, the natural substrate to PHD can be prevented (Hirota 2021). Daprodustat (Caltabiano et al. 2018; Dhillon 2020), together with Enarodustat (Fukui et al. 2019), Molidustat (Lentini et al. 2020), Roxadustat (Dhillon 2019) and Vadadustat (Markham 2020), all competitive inhibitors of 2-OG which inhibits PHD, have completed Phase III trials and are currently approved for the treatment of renal anemia in Japan, being used in clinical practice as of 2021 (Yap et al. 2021).

NIH/3T3 cells were cultured for 48 hours with variable concentrations of Daprodustat (FIG. 18A), Enarodustat (FIG. 19A), Molidustat (FIG. 20A), Vadadustat (FIG. 21A) and Roxadustat (FIG. 22A), followed by immunofluorescence and labelling for collagen type 1.

Treatment of NIH/3T3 cells with Daprodustat results in increased production of Collagen type-1 without causing cell toxicity—Multiple comparisons were performed by ordinary one-way ANOVA followed by Dunnett's post-hoc statistical hypothesis, show no significant difference between the number of cells treated with Daprodustat (FIG. 18A) compared to untreated cells, except for lower number of cells treated with 0.08 μM Daprodustat (p<0.0001), suggesting no toxicity for Daprodustat (FIG. 18B). Moreover, the present inventors show a positive trend for collagen type 1 expression while raising Daprodustat concentrations, with significant increase for treated cells with 0.31-5 μM Daprodustat (p<0.0001) compared to untreated cells (FIG. 18C). Representative images show the extracellular labelling of collagen type 1 (FIG. 18D).

Treatment of NIH/3T3 cells with Enarodustat results in increased production of Collagen type-1 without causing cell toxicity—Moreover, same statistical analysis show no significant difference between the number of cells treated with Enarodustat (FIG. 19A) compared to untreated cells (p>0.05), suggesting no toxicity for Enarodustat (FIG. 19B). In addition, the present inventors also show a positive trend for collagen type 1 expression while raising Enarodustat concentrations, with a significant increase for treated cells with 0.31-5 μM Enarodustat (0.31 μM; p<0.0001, 0.63 μM; p<0.001, 1.25-5 μM; p<0.0001) compared to untreated cells (FIG. 19C). Representative images show the extracellular labelling of collagen type 1 (FIG. 19D).

Treatment of NIH/3T3 cells with Molidustat results in significant increases in Collagen type-1 expression at concentration range of 0.63-5 μM Molidustat, yet with some cell toxicity—Treatment of NIH/3T3 cells with Molidustat (FIG. 20A) show that the number of cells was significantly lower for cells treated with Molidustat compared to untreated cells (FIG. 20B; 0.16 μM; p<0.0001, 0.31 μM; p<0.01, 0.63 μM; p<0.05, 1.25-5 μM; p<0.0001), suggesting a toxic effect of Molidustat on NIH/3T3 cells. However, there was a positive trend for collagen type 1 expression while raising Molidustat concentrations, with significant increase for treated cells with 0.63-5 μM Molidustat (p<0.0001) compared to untreated cells (FIG. 20C). Representative images show the extracellular labelling of collagen type 1 (FIG. 20D).

Treatment of NIH/3T3 cells with Vadadustat results in increased production of Collagen type-1 without causing cell toxicity—In addition, for the treatment with Vadadustat (FIG. 21A), the number of cells was significantly higher for cells treated with Vadadustat compared to untreated cells, suggesting no toxicity for Vadadustat (FIG. 21B; 0.02 μM; p<0.01, 0.04-0.08 μM; p<0.05, 0.16 μM; p<0.0001, 0.31 μM; p<0.001, 0.63-2.5 μM; p<0.0001, 5 μM; p<0.001). Moreover, the present inventors show higher expression of collagen type 1 while raising Vadadustat concentrations, with a significant increase for cells treated with Vadadustat (0.02 μM; p<0.01, 0.04 μM; p<0.0001, 0.08 μM; p<0.05, 0.16 μM; p<0.0001, 0.31 μM; p<0.001, 0.63 μM; p<0.0001, 1.25 μM; p<0.001, 2.5 μM; p<0.0001, 5 μM; p<0.001) compared to untreated cells (FIG. 21C). Representative images show the extracellular labelling of collagen type 1 (FIG. 21D).

Treatment of NIH/3T3 cells with Roxadustat results in increased production of Collagen type-1 without causing cell toxicity—Lastly, for the treatment with Roxadustat (FIG. 22A), the number of cells was significantly higher for cells treated with 0.02 μM (p<0.01) and 0.08-2.5 μM Roxadustat (0.08 μM; p<0.05, 0.16-0.31 μM; p<0.01, 0.63 μM; p<0.001, 1.25-2.5 μM; p<0.0001) compared to untreated cells, suggesting no toxicity for Roxadustat (FIG. 22B). Moreover, we show a positive trend for collagen type 1 expression while raising Roxadustat concentrations, with significant increase for treated cells with 0.08-5 μM Roxadustat (0.08 μM; p<0.05, 0.16 μM; p<0.001, 0.31-5 μM; p<0.0001) compared to untreated cells (FIG. 22C). Representative images show the extracellular labelling of collagen type 1 (FIG. 22D).

Taken together, the results presented in the Examples section above demonstrate that when small molecules like ML228 are used to activate HIF1α, or other molecules such as peptides, iron chelators or specific inhibitors are used to directly inhibit HIF-PHD protein activity, or the interaction between HIF and PHD, they lead to the stabilization of HIF-1 (e.g., as schematically shown in FIG. 7) in an amount which is sufficient and effective in enhancing Collagen levels.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### CITED REFERENCES (ADDITIONAL REFERENCES ARE CITED IN TEXT)

ADDIN Mendeley Bibliography CSL_BIBLIOGRAPHY Caltabiano, Stephen et al. 2018. “The Drug Interaction Potential of Daprodustat When Coadministered with Pioglitazone, Rosuvastatin, or Trimethoprim in Healthy Subjects.” Pharmacology Research & Perspectives 6(2): e00327.
Chan, Mun Chiang et al. 2015. “Potent and Selective Triazole-Based Inhibitors of the Hypoxia-Inducible Factor Prolyl-Hydroxylases with Activity in the Murine Brain.” PLoS ONE 10(7): 1-17.
Cheng-Jun Hu et al., 2007; Molecular Biology of the Cell Vol. 18, 4528-4542;
Dhillon, Sohita. 2019. “Roxadustat: First Global Approval.” Drugs 79(5): 563-72.
Dhillon, Sohita. 2020. “Daprodustat: First Approval.” Drugs 80(14): 1491-97.
Figg, William D. et al. 2021. “Structural Basis of Prolyl Hydroxylase Domain Inhibition by Molidustat.” ChemMedChem 16(13): 2082-88.
Fukui, Kenji et al. 2019. “JTZ-951 (Enarodustat), a Hypoxia-Inducible Factor Prolyl Hydroxylase Inhibitor, Stabilizes HIF-α Protein and Induces Erythropoiesis without Effects on the Function of Vascular Endothelial Growth Factor.” European Journal of Pharmacology 859: 172532.
Hirota, Kiichi. 2021. “HIF-α Prolyl Hydroxylase Inhibitors and Their Implications for Biomedicine: A Comprehensive Review.” Biomedicines 9(5).
Hirota, Kiichi, and Gregg L. Semenza. 2005. “Regulation of Hypoxia-Inducible Factor 1 by Prolyl and Asparaginyl Hydroxylases.” Biochemical and Biophysical Research Communications 338(1): 610-16.
Karsenty, Gerard, and Rang Woon Park. 1995. “Regulation of Type I Collagen Genes Expression.” International Reviews of Immunology 12(2-4): 177-85.
Lentini, Silvia et al. 2020. “Effects of Oral Iron and Calcium Supplement on the Pharmacokinetics and Pharmacodynamics of Molidustat: An Oral HIF-PH Inhibitor for the Treatment of Renal Anaemia.” European Journal of Clinical Pharmacology 76(2): 185-97.
Lifshits, Lucia Adriana et al. 2022. “Enhancement of Collagen-I Levels in Human Gingival Fibroblasts by Small Molecule Activation of HIF-1α.” Journal of Agricultural and Food Chemistry.
Markham, Anthony. 2020. “Vadadustat: First Approval.” Drugs 80(13): 1365-71.
Schofield, Christopher J., and Peter J. Ratcliffe. 2004. “Oxygen Sensing by HIF Hydroxylases.” Nature Reviews Molecular Cell Biology 5(5): 343-54.
Theriault, Jimmy R. et al. 2012. “Discovery of a New Molecular Probe ML228: An Activator of the Hypoxia Inducible Factor (HIF) Pathway.” Bioorganic and Medicinal Chemistry Letters 22(1): 76-81.
Wilkins, Sarah E., Martine I. Abboud, Rebecca L. Hancock, and Christopher J. Schofield. 2016. “Targeting Protein-Protein Interactions in the HIF System.” ChemMedChem 11(8): 773-86.
Yap, Desmond Y. H. et al. 2021. “Recommendations by the Asian Pacific Society of Nephrology (APSN) on the Appropriate Use of HIF-PH Inhibitors.” Nephrology 26(2): 105-18.
Yeh, Tzu Lan et al. 2017. “Molecular and Cellular Mechanisms of HIF Prolyl Hydroxylase Inhibitors in Clinical Trials.” Chemical Science 8(11): 7651-68.

What is claimed is:

1. An ex vivo method of increasing collagen production in cells comprising contacting the cells with an agent that increases an amount of Hypoxia-inducible factor-1 (HIF-1) in the cells, said amount being selected to cause an increase in the amount of collagen production in said cells.

2. A cell culture comprising non-human cells and a culture medium which comprises an activator of Hypoxia-inducible factor-1 (HIF-1).

3. A method of inducing tissue regeneration or cell repair in a subject in need thereof comprising administering to the subject an HIF activator in an amount which brings about an up-regulation of collagen, thereby inducing tissue regeneration or cell repair.

4. The method of claim 1, wherein said agent that increases said amount of said HIF-1 is an activator of said HIF-1.

5. The method of claim 3, wherein said method is a non-invasive method.

6. The method of claim 1, wherein said contacting comprises culturing said cells in a medium comprising an activator of said HIF-1.

7. The cell culture of claim 2, wherein said activator of said HIF-1 is provided in an amount which brings about an up-regulation of collagen.

8. The method of claim 4, wherein said activator of said HIF-1 comprises a peptide agent.

9. The method of claim 8, wherein said peptide comprises at least 15 and no more than 25 consecutive amino acid residues of the HIF-1α polypeptide set forth by SEQ ID NO: 5.

10. The method of claim 8, wherein said peptide comprises at least 15 and no more than 25 consecutive amino acid residues of the EPAS1 (HIF-2α) polypeptide set forth by SEQ ID NO: 6.

11. The method of claim 9, wherein said peptide is set forth by SEQ ID NO: 4.

12. The method of claim 10, wherein said peptide is set forth by SEQ ID NO: 2.

13. The method of claim 9, wherein said peptide further comprises a cell penetrating peptide.

14. The method of claim 13, wherein said cell penetrating peptide is set forth by SEQ ID NO: 1.

15. The method of claim 4, wherein said activator of said HIF-1 is a small molecule agent, wherein said small molecule is selected from the group consisting of ML228, an iron chelator, an analogue of 2-oxoglutarate (2-OG), Roxadustat (FG-4592), Daprodustat, Molidustat, Enarodustat (JTZ-951) and Vadadustat, or derivatives thereof.

16. The method of claim 4, wherein said activator of said HIF-1 comprises an agent which downregulates expression of HIF-PHD.

17. The method of claim 16, wherein said agent is an RNAi molecule.

18. The method of claim 1, wherein said cells comprise human cells.

19. A method of generating collagen comprising:
(a) increasing collagen production according to the method of claim 1; and
(b) isolating said collagen from said cells or from said medium.

20. A method of generating a food product comprising:
(a) increasing collagen production according to the method of claim 19;
(b) isolating said cells from said medium;
(c) combining said cells or said collagen isolated from said cells with a food or feed product.
